(19) United States
(12) Patent Application Publication
(10) Pub. No.: US 2008/0161278 A1
(43) Pub. Date: Jul. 3, 2008

(54) 2-Azetidinyl-4-(1H-Pyrazol-3-Ylamino) Pyrimidines as Inhibitors of Insulin-Like Growth Factor-1 Receptor Activity

(75) Inventors: Andrew Peter Thomas, Cheshire (GB); Thorsten Nowak, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/909,243

(22) PCT Filed: Mar. 21, 2006
(86) PCT No.: PCT/GB2006/001013

§ 371 Date: Sep. 20, 2007

(30) Foreign Application Priority Data
Mar. 23, 2005 (GB) 0505913.4
Dec. 21, 2005 (GB) 0525952.8

(51) Int. Cl. A61K 31/506; C07D 403/14

(52) U.S. Cl. 514/210.2; 544/324; 544/60; 544/122; 544/295

(57) ABSTRACT

A compound of formula (I): wherein the substituents are as defined in the text for use in inhibiting insulin-like growth factor 1 receptor activity in a warm blooded animal such as man.

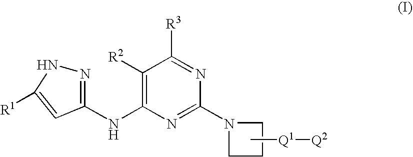

The invention concerns certain novel pyrimidine derivatives, or pharmaceutically-acceptable salts thereof, which possess anti-tumour activity and are accordingly useful in methods of treatment of the human or animal body. The invention also concerns processes for the manufacture of the pyrimidine derivatives, to pharmaceutical compositions containing them and to their use in therapeutic methods, for example in the manufacture of medicaments for use in the prevention or treatment of solid tumour disease in a warm-blooded animal such as man.

The insulin-like growth factor (IGF) axis consists of ligands, receptors, binding proteins and proteases. The two ligands, IGF-I and IGF-II, are mitogenic peptides that signal through interaction with the type 1 insulin-like growth factor receptor (IGF-1R), a hetero-tetrameric cell surface receptor. Binding of either ligand stimulates activation of a tyrosine kinase domain in the intracellular region of the β-chain and results in phosphorylation of several tyrosine residues resulting in the recruitment and activation of various signalling molecules. The intracellular domain has been shown to transmit signals for mitogenesis, survival, transformation, and differentiation in cells. The structure and function of the IGF-1R has been reviewed by Adams et al (Cellular and Molecular Life Sciences, 57, 1050-1093, 2000). The IGF-IIR (also known as mannose 6-phosphate receptor) has no such kinase domain and does not signal mitogenesis but may act to regulate ligand availability at the cell surface, counteracting the effect of the IGF-1R. The IGF binding proteins (IGFBP) control availability of circulating IGF and release of IGF from these can be mediated by proteolytic cleavage. These other components of the IGF axis have been reviewed by Collett-Solberg and Cohen (Endocrine, 12, 121-136, 2000).

There is considerable evidence linking IGF signalling with cellular transformation and the onset and progression of tumours. IGF has been identified as the major survival factor that protects from oncogene induced cell death (Harrington et al, EMBO J, 13, 3286-3295, 1994). Cells lacking IGF-1R have been shown to be refractory to transformation by several different oncogenes (including SV40T antigen and ras) that efficiently transform corresponding wild-type cells (Sell et al., Mol. Cell Biol., 14, 3604-12, 1994). Upregulation of components of the IGF axis has been described in various tumour cell lines and tissues, particularly tumours of the breast (Surmacz, Journal of Mammary Gland Biology & Neoplasia, 5, 95-105, 2000), prostate (Djavan et al, World J. Urol., 19, 225-233, 2001, and O'Brien et al, Urology, 58, 1-7, 2001) and colon (Guo et al, Gastroenterology, 102, 1101-1108, 1992). Conversely, IGF-IIR has been implicated as a tumour suppressor and is deleted in some cancers (DaCosta et al, Journal of Mammary Gland Biology & Neoplasia, 5, 85-94, 2000). There are a growing number of epidemiological studies linking increased circulating IGF (or increased ratio of IGF-1 to IGFBP3) with cancer risk (Yu and Rohan, J. Natl. Cancer Inst., 92, 1472-1489, 2000). Transgenic mouse models also implicate IGF signalling in the onset of tumour cell proliferation (Lamm and Christofori, Cancer Res. 58, 801-807, 1998, Foster et al, Cancer Metas. Rev., 17, 317-324, 1998, and DiGiovanni et al, Proc. Natl. Acad. Sci., 97, 3455-3460, 2000).

Several in vitro and in vivo strategies have provided the proof of principal that inhibition of IGF-1R signalling reverses the transformed phenotype and inhibits tumour cell growth. These include neutralizing antibodies (Kalebic et al Cancer Res., 54, 5531-5534, 1994), antisense oligonucleotides (Resnicoff et al, Cancer Res., 54, 2218-2222, 1994), triple-helix forming oligonucleotides (Rinninsland et al, Proc. Natl. Acad. Sci., 94, 5854-5859, 1997), antisense mRNA (Nakamura et al, Cancer Res., 60, 760-765, 2000) and dominant negative receptors (D'Ambrosio et al., Cancer Res., 56, 4013-4020, 1996). Antisense oligonucleotides have shown that inhibition of IGF-1R expression results in induction of apoptosis in cells in vivo (Resnicoff et al, Cancer Res., 55, 2463-2469, 1995) and have been taken into man (Resnicoff et al, Proc. Amer. Assoc. Cancer Res., 40 Abs 4816, 1999). However, none of these approaches is particularly attractive for the treatment of major solid tumour disease.

Since increased IGF signalling is implicated in the growth and survival of tumour cells, and blocking IGF-1R function can reverse this, inhibition of the IGF-1R tyrosine kinase domain is an appropriate therapy by which to treat cancer. In vitro and in vivo studies with the use of dominant-negative IGF-1R variants support this. In particular, a point mutation in the ATP binding site which blocks receptor tyrosine kinase activity has proved effective in preventing tumour cell growth (Kulik et al, Mol. Cell. Biol., 17, 1595-1606, 1997). Several pieces of evidence imply that normal cells are less susceptible to apoptosis caused by inhibition of IGF signalling, indicating that a therapeutic margin is possible with such treatment (Baserga, Trends Biotechnol., 14, 150-2, 1996).

There are few reports of selective IGF-1R tyrosine kinase inhibitors. Parrizas et al. described tyrphostins that had some efficacy in vitro and in vivo (Parrizas et al., Endocrinology, 138:1427-33 (1997)). These compounds were of modest potency and selectivity over the insulin receptor. Telik Inc. have described heteroaryl-aryl ureas which have selectivity over insulin receptors but potency against tumour cells in vitro is still modest (Published PCT Patent Application No. WO 00/35455). Novartis have disclosed a pyrazolopyrimidine compound (known as NVP-AEW541), which is reported to inhibit IGF-1R tyrosine kinase (Garcia-Echeverria et al., Cancer Cell, 5:231-39 (2004)). Axelar have described podophyllotoxin derivatives as specific IGFR tyrosine kinase inhibitors (Vasilcanu et al., Oncogene, 23: 7854-62 (2004)) and Aventis have described cyclic urea derivatives and their use as IGF-1R tyrosine kinase inhibitors (WO 2004/070050).

Additionally, several anti-IGFR antibodies are reported to block receptor signalling and show inhibition of tumour growth in animal models (Cohen et al., Clin. Canc. Res., 11: 2063-73 (2005); Burtrum et al., Canc. Res., 63: 8912-21 (2003); Goetsch et al., Int. J. Cancer, 113: 316-28 (2005) and Maloney et al., Canc. Res., 63: 5073-83 (2003)).

Pyrimidine derivatives substituted at the 2- and 4-positions by a substituted amino group having IGF-IR tyrosine kinase inhibitory activity are described in WO 03/048133. Compounds in which the nitrogen atom of the amino substituent forms part of a heterocyclic ring are not disclosed.

Substituted pyrimidine derivatives are also described in WO 00/39101, WO 2004/056786, WO 2004/080980 and WO 2004/048365, but none of these documents describe pyrimidine derivatives having a N-linked azetidine ring at the 2-position on the pyrimidine ring.

WO 02/50065 discloses that certain pyrazolyl-amino substituted pyrimidine derivatives have protein kinase inhibitory activity, especially as inhibitors of Aurora-2 and glycogen synthase kinase-3 (GSK-3), and are useful for treating diseases such as cancer, diabetes and Alzheimer's disease. The compounds disclosed may have a substituted amino substituent at the 2-position of the pyrimidine ring but again there is no disclosure of compounds in which the nitrogen atom of the amino substituent forms part of a heterocyclic ring.

WO 01/60816 discloses that certain substituted pyrimidine derivatives have protein kinase inhibitory activity. There is no disclosure in WO 01/60816 of pyrimidine derivatives having a pyrazolyl-amino substituent at the 4-position on the pyrimidine ring and a N-linked azetidine ring at the 2-position on the pyrimidine ring.

Pyrazolyl-amino substituted pyrimidine derivatives having Aurora-2 and glycogen synthase kinase-3 (GSK-3) inhibitory activity in which the 2-position of the pyrimidine ring is substituted by an N-linked heterocyclic ring are disclosed generically in WO 02/22601, WO 02/22602, WO 02/22603, WO 02/22604, WO 02/22605, WO 02/22606, WO 02/22607 and WO 02/22608. There is no disclosure of compounds in which the N-linked heterocyclic ring is an azetidine ring.

WO 2005/040159 (International patent application number PCT/GB2004/004307) discloses certain pyrimidine derivatives and their use in modulating insulin-like growth factor 1 receptor activity. There is no disclosure of pyrimidine compounds that contain an azetidine ring at the 2-position of the pyrimidine ring.

We have now found that certain pyrimidine compounds that contain a substituted azetidine ring at the 2-position on the pyrimidine ring possess potent anti-tumour activity. Without wishing to imply that the compounds disclosed in the present invention possess pharmacological activity only by virtue of an effect on a single biological process, it is believed that the compounds provide an anti-tumour effect by way of inhibition of IGF-1R tyrosine kinase activity.

According to a first aspect of the invention, there is provided a compound of formula (I):

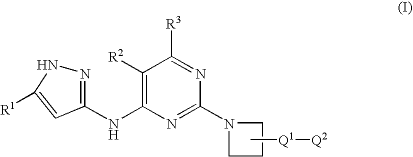

wherein:

R¹ is selected from a (C1-C6)alkyl, (C3-C8)cycloalkyl or (C3-C8)cycloalkyl(C1-C6)alkyl group, each of which groups may be optionally substituted by one or more substituents independently selected from halogeno and (C1-C6)alkoxy;

R² is selected from hydrogen, halogeno and trifluoromethyl;

R³ is selected from hydrogen, hydroxy and halogeno, or from a (C1-C6)alkyl, (C2-C6)alkenyl, (C2-C6)alkynyl, (C3-C8)cycloalkyl, (C3-C8)cycloalkyl(C1-C6)alkyl, (C1-C6)alkoxy, (C3-C8)cycloalkyl(C1-C6)alkoxy, (C1-C6)alkylcarbonyl, (C3-C8)cycloalkylcarbonyl, (C3-C8)cycloalkyl(C 1-C6)alkylcarbonyl, (C1-C6)alkoxycarbonyl, amino, (C1-C6)alkylamino, di-[(C1-C6)alkyl]amino, (C3-C8)cycloalkylamino, (C3-C8)cycloalkyl(C1-C6)alkylamino, (C1-C6)alkoxyamino, carbamoyl, (C1-C6)alkylcarbamoyl, di-[(C1-C6)alkyl]carbamoyl, —C(O)R^{3b}, —OR^{3b}, —SR^{3b}, —NHR^{3b}, —N[(C1-C6)alkyl]R^{3b}, —S(O)ₘR^{3a} or —N(R^{3c})C(O)R^{3a} group, wherein R^{3a} is selected from a (C1-C6)alkyl, (C3-C8)cycloalkyl, (C3-C8)cycloalkyl(C1-C6)alkyl or (C1-C6)alkoxy group, m is 0, 1 or 2, R^{3b} is a saturated monocyclic 4-, 5- or 6-membered heterocyclic ring comprising at least one ring heteroatom selected from nitrogen, oxygen and sulfur and R^{3c} is selected from hydrogen and (C1-C6)alkyl,

or R³ is a saturated monocyclic 5- or 6-membered heterocyclic ring comprising at least one ring heteroatom selected from nitrogen, oxygen and sulfur,

or R³ is a 5- or 6-membered heteroaromatic ring comprising at least one ring heteroatom selected from nitrogen, oxygen and sulfur,

or R³ is a 2,7-diazaspiro[3.5]nonane group,

each of which groups or rings within R³ may be optionally substituted by one or more substituents independently selected from (C1-C6)alkyl, (C1-C6)alkoxy, (C1-C6)alkoxy(C1-C6)alkyl, (C1-C6)alkoxy(C1-C6)alkoxy, halogeno, hydroxy, trifluoromethyl, tri-[(C1-C4)alkyl]silyl, cyano, amino, (C1-C6)alkylamino, di-[(C1-C6)alkyl]amino, (C3-C8)cycloalkylamino, (C3-C6)cycloalkyl(C1-C3)alkylamino, amino(C1-C6)alkyl, (C1-C6)alkylamino(C1-C6)alkyl, di-[(C1-C6)alkyl]amino(C1-C6)alkyl, (C3-C8)cycloalkylamino(C1-C6)alkyl, (C3-C6)cycloalkyl(C1-C3)alkylamino(C1-C6)alkyl, (C1-C6)alkoxycarbonyl, carbamoyl, (C1-C6)alkylcarbamoyl, di-[(C1-C6)alkyl]carbamoyl, (C1-C6)alkylthio, (C1-C6)alkylsulfonyl, (C1-C6)alkylsulfinyl, (C1-C6)alkanoyl, an alkanoylamino group —N(R^{3d})C(O)R^{3e} wherein R^{3d} is selected from hydrogen and (C1-C6)alkyl and R^{3e} is selected from a (C1-C6)alkyl, (C3-C8)cycloalkyl, (C3-C8)cycloalkyl(C1-C6)alkyl or (C1-C6)alkoxy group, or a saturated monocyclic 3-, 4-, 5-, 6- or 7-membered ring, which ring may optionally comprise one or more heteroatoms selected from nitrogen, oxygen and sulfur, any of which substituents may be optionally substituted by one or more (C1-C4)alkyl, hydroxy or cyano groups;

Q¹ is a 5- or 6-membered heteroaromatic ring comprising at least one ring heteroatom selected from nitrogen, oxygen and sulfur, which ring is substituted by Q² and is optionally substituted, on any available ring atom, by one or more further substituents independently selected from (C1-C6)alkyl and (C1-C6)alkoxy (either of which (C1-C6)alkyl and (C1-C6)alkoxy substituent groups may be optionally substituted by one or more substituents independently selected from halogeno, amino, hydroxy and trifluoromethyl), halogeno, nitro, cyano, —NR⁴R⁵, carboxy, hydroxy, (C2-C6)alkenyl, (C3-C8)cycloalkyl, (C3-C8)cycloalkyl(C1-C6)alkyl, (C1-C4)alkoxycarbonyl, (C1-C4)alkylcarbonyl, (C2-C6)alkanoylamino, phenylcarbonyl, —S(O)ₚ(C1-C4)alkyl, —C(O)NR⁶R⁷ and —SO₂NR⁸R⁹, wherein R⁴, R⁵, R⁶, R⁷, R⁸ and R⁹ are each independently selected from hydrogen and (C1-C6)alkyl, or R⁴ and R⁵, or R⁶ and R⁷, or R⁸ and R⁹, when taken together with the nitrogen atom to which they are attached, may each independently form a saturated heterocyclic ring and p is 0, 1 or 2;

Q² is selected from a (C1-C6)alkyl, (C3-C6)cycloalkyl or (C3-C6)cycloalkyl(C1-C6)alkyl group or a saturated or unsaturated 5- or 6-membered monocyclic ring which may comprise at least one ring heteroatom selected from nitrogen, oxygen and sulfur, and wherein Q² is optionally substituted by one or more substituents independently selected from (C1-C6)alkyl and (C1-C6)alkoxy (either of which (C1-C6)alkyl and (C1-C6)alkoxy substituent groups may be optionally substituted by one or more substituents independently selected from halogeno, amino, hydroxy and trifluoromethyl), halogeno, nitro, cyano, —NR¹⁰R¹¹, carboxy, hydroxy, (C2-C6)alkenyl, (C3-C8)cycloalkyl, (C1-C6)alkoxycarbonyl, (C1-C6)alkylcarbonyl, (C2-C6)alkanoylamino, phenylcarbonyl, —S(O)ₙ(C1-C6)alkyl, —C(O)NR¹²R¹³ and —SO₂NR¹⁴R¹⁵, wherein R¹⁰, R¹¹, R¹², R¹³, R¹⁴ and R¹⁵ are each independently selected from hydrogen and (C1-C6)alkyl, or R¹⁰ and R¹¹, or R¹² and R¹³, or R¹⁴ and R¹⁵, when taken together with the nitrogen atom to which they are attached, may each independently form a saturated heterocyclic ring and n is 0, 1 or 2;

and wherein any saturated monocyclic ring optionally bears 1 or 2 oxo or thioxo substituents;

or a pharmaceutically-acceptable salt thereof.

In this specification, unless otherwise indicated, the term “alkyl” when used alone or in combination, includes both straight chain and branched chain alkyl groups, such as propyl, isopropyl and tert-butyl. However, references to individual alkyl groups such as “propyl” are specific for the straight-chain version only and references to individual branched-chain alkyl groups such as “isopropyl” are specific for the branched-chain version only. A (C1-C6)alkyl group has from one to six carbon atoms including methyl, ethyl, n-propyl, isopropyl, tert-butyl, n-pentyl, n-hexyl and the like. References to “(C1-C4)alkyl” will be understood accordingly to mean a straight or branched chain alkyl moiety having from one to four carbon atoms.

An analogous convention applies to other generic terms, for example, the terms “(C1-C6)alkoxy” and “(C1-C4)alkoxy”, when used alone or in combination, will be understood to refer to straight or branched chain groups having from one to six, or from one to four, carbon atoms respectively and include such groups as methoxy, ethoxy, propoxy, isopropoxy and butoxy.

A “(C2-C6)alkenyl” group includes both straight chain and branched chain alkenyl groups having from two to six carbon atoms, such as vinyl, isopropenyl, allyl and but-2-enyl. Similarly, a “(C2-C6)alkynyl” group includes both straight chain and branched chain alkynyl groups having from two to six carbon atoms, such as ethynyl, 2-propynyl and but-2-ynyl.

The term “(C3-C8)cycloalkyl”, when used alone or in combination, refers to a saturated alicyclic moiety having from three to eight carbon atoms and includes, for example, cyclopropyl, cyclobutyl, cyclopentyl, cyclohexyl and cycloheptyl. References to “(C3-C6)cycloalkyl” will be understood accordingly to mean a saturated alicyclic moiety having from three to six carbon atoms, representative examples of which are listed above.

As used herein, the term “halogeno” includes fluoro, chloro, bromo and iodo.

The term “optionally substituted” is used herein to indicate optional substitution by the group or groups specified at any suitable available position.

A “heteroatom” is a nitrogen, sulfur or oxygen atom. Where rings include nitrogen atoms, these may be substituted as necessary to fulfil the bonding requirements of nitrogen or they may be linked to the rest of the structure by way of the nitrogen atom. Nitrogen atoms may also be in the form of N-oxides. Sulfur atoms may be in the form of S, S(O) or SO₂.

Suitable values for the generic radicals referred to above include those set out below.

A suitable value for a substituent on R³ when it is a “saturated monocyclic 3-, 4-, 5-, 6- or 7-membered ring, which ring may optionally comprise one or more heteroatoms selected from nitrogen, oxygen and sulfur” is a carbocyclic ring containing 3, 4, 5, 6 or 7 atoms (that is an alicyclic ring having ring carbon atoms only) or a heterocyclic ring containing 3, 4, 5, 6 or 7 atoms of which at least one is a heteroatom selected from nitrogen, oxygen and sulfur. When the “saturated monocyclic 3-, 4-, 5-, 6- or 7-membered ring, which ring may optionally comprise one or more heteroatoms selected from nitrogen, oxygen and sulfur” is a heterocyclic ring, the heterocyclic ring suitably contains from one to four (for example, from one to three, or one or two) heteroatoms independently selected from nitrogen, oxygen and sulfur. Unless specified otherwise, the heterocyclic ring may be carbon or nitrogen linked. Examples of suitable saturated monocyclic 3-, 4-, 5-, 6- or 7-membered carbocyclic rings include cyclopropyl, cyclobutyl, cyclopentyl, cyclohexyl and cycloheptyl. Examples of suitable saturated monocyclic 3-, 4-, 5-, 6- or 7-membered heterocyclic rings include oxiranyl, azetidinyl, dioxanyl, trioxanyl, oxepanyl, dithianyl, trithianyl, oxathianyl, thiomorpholinyl, pyrrolidinyl, piperidinyl, imidazolidinyl, morpholinyl, tetrahydrofuranyl, tetrahydropyranyl and piperazinyl (particularly azetidinyl, pyrrolidinyl, piperidinyl, morpholinyl, tetrahydrofuranyl, tetrahydropyranyl and piperazinyl). A saturated heterocyclic ring that bears 1 or 2 oxo or thioxo substituents may, for example, be 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl.

A suitable value for R³b when it is a “saturated monocyclic 4-, 5- or 6-membered heterocyclic ring comprising at least one ring heteroatom selected from nitrogen, oxygen and sulfur” is a heterocyclic ring containing four, five or six ring atoms, representative examples of which are listed above.

A suitable value for R³ when it is a “saturated monocyclic 5- or 6-membered heterocyclic ring comprising at least one ring heteroatom selected from nitrogen, oxygen and sulfur” is a heterocyclic ring containing five or six ring atoms, representative examples of which are listed above.

A suitable value for Q¹ or for R³ when it is a “5- or 6-membered heteroaromatic ring comprising at least one ring heteroatom selected from nitrogen, oxygen and sulfur” is a fully unsaturated, aromatic monocyclic ring containing five or six atoms of which at least one is a heteroatom selected from nitrogen, oxygen and sulfur, which ring may, unless otherwise specified, be carbon or nitrogen linked. Particularly, the 5- or 6-membered heteroaromatic ring may contain from one to four (for example, from one to three, or one or two) heteroatoms independently selected from nitrogen, oxygen and sulfur. Examples of such heteroaromatic rings include pyridyl, imidazolyl, isoxazolyl, pyrazolyl, furyl, pyrazinyl, pyridazinyl, pyrimidinyl, pyrrolyl, thiazolyl, oxazolyl, oxadiazolyl, isothiazolyl, triazolyl, tetrazolyl and thienyl.

A suitable value for Q² when it is a “saturated or unsaturated 5- or 6-membered monocyclic ring which may comprise at least one ring heteroatom selected from nitrogen, oxygen and sulfur” is a saturated or fully or partially unsaturated monocyclic ring containing five or six atoms of which optionally at least one is a heteroatom selected from nitrogen, oxygen and sulfur, which ring may, unless otherwise specified, be carbon or nitrogen linked. The ring may have alicyclic or aromatic properties. An aromatic monocyclic ring may be aryl (such as phenyl) or heteroaromatic, representative examples of which are listed above.

When R³ is a 2,7-diazaspiro[3.5]nonane group, it is preferably linked to the pyrimidine ring via a nitrogen atom, particularly via. the nitrogen atom at the 7-position. When the 2,7-diazaspiro[3.5]nonane group carries a substituent, this may be at any available carbon or nitrogen atom, for example at any nitrogen atom that is not attached to the pyrimidine ring. A particular substituted 2,7-diazaspiro[3.5]nonane group may, for example, be 2-(tert-butoxycarbonyl)-2,7-diazaspiro[3.5]nonane.

Where R⁴ and R⁵, or R⁶ and R⁷, or R⁸ and R⁹, or R¹⁰ and R¹¹, or R¹² and R¹³, or R¹⁴ and R¹⁵ form a saturated heterocyclic ring, the only heteroatom present is the nitrogen atom to which R⁴ and R⁵, or R⁶ and R⁷, or R⁸ and R⁹, or R¹⁰ and R¹¹, or R¹² and R¹³, or R¹⁴ and R¹⁵ are attached. The saturated heterocyclic ring is preferably a 4-, 5-, 6- or 7-membered ring, including the nitrogen atom to which R⁴ and R⁵, or R⁶ and R⁷, or R⁸ and R⁹, or R¹⁰ and R¹¹, or R¹² and R¹³, or R¹⁴ and R¹⁵ are attached.

For the avoidance of any doubt the nitrogen atom in the azetidine ring to which the pyrimidine group is attached is not quaternised; namely the pyrimidine group is attached to the nitrogen atom in the azetidine ring via. substitution of an NH group in the azetidine ring.

The azetidine ring may be substituted at any substitutable position in the ring by Q¹. Preferably, the azetidine ring is substituted by Q¹ at a ring atom adjacent to the nitrogen atom linking the azetidine ring to the pyrimidine ring of the compounds of the invention.

Suitable values for any of the substituents herein, for example the ‘R’ groups (R¹ to R¹⁸, R^{3a}, R^{3b}, R^{3c}, R^{3d} or R^{3e}) or for various groups within a Q¹ or Q² group include:

| | |
|---|---|
| for halogeno: | fluoro, chloro, bromo and iodo; |
| for (C1-C6)alkyl: | methyl, ethyl, propyl, isopropyl, tert-butyl, n-pentyl |
| | and n-hexyl; |
| for (C2-C6)alkenyl: | vinyl, isopropenyl, allyl and but-2-enyl; |
| for (C2-C6)alkynyl: | ethynyl, 2-propynyl and but-2-ynyl; |
| for (C1-C6)alkoxy: | methoxy, ethoxy, propoxy, isopropoxy and butoxy; |
| for (C1-C6)alkoxy(C1-C6)alkoxy: | methoxymethoxy, methoxyethoxy, ethoxymethoxy, |
| | propoxymethoxy and butoxymethoxy; |
| for (C1-C6)alkoxy(C1-C6)alkyl: | methoxymethyl, methoxyethyl, ethoxymethyl, |
| | propoxymethyl and butoxymethyl; |
| for tri-[(C1-C4)alkyl]silyl | trimethylsilyl, triethylsilyl, dimethyl-ethylsilyl and |
| | methyl-diethylsilyl; |
| for (C1-C6)alkylthio: | methylthio, ethylthio and propylthio; |
| for (C1-C6)alkylamino: | methylamino, ethylamino, propylamino, |
| | isopropylamino and butylamino; |
| for di-[(C1-C6)alkyl]amino: | dimethylamino, diethylamino, N-ethyl- |
| | N-methylamino and diisopropylamino; |
| for amino(C1-C6)alkyl: | aminomethyl, aminoethyl, aminopropyl and |
| | aminobutyl; |
| for (C1-C6)alkylamino(C1-C6)alkyl: | methylaminomethyl, methylaminoethyl, |
| | methylaminopropyl, ethylaminomethyl, |
| | ethylaminoethyl, propylaminomethyl, |
| | isopropylaminoethyl and butylaminomethyl; |
| for di-[(C1-C6)alkyl]amino(C1-C6)alkyl: | dimethylaminomethyl, dimethylaminoethyl, |
| | dimethylaminobutyl, diethylaminomethyl, |
| | diethylaminoethyl, diethylaminopropyl, N-ethyl- |
| | N-methylaminomethyl, N-ethyl- |
| | N-methylaminomethyl and diisopropylaminoethyl; |
| for (C1-C6)alkylcarbonyl: | methylcarbonyl, ethylcarbonyl, propylcarbonyl and |
| | tert-butylcarbonyl; |
| for (C1-C6)alkoxycarbonyl: | methoxycarbonyl, ethoxycarbonyl, propoxycarbonyl |
| | and tert-butoxycarbonyl; |
| for (C1-C6)alkylcarbamoyl: | N-methylcarbamoyl, N-ethylcarbamoyl and |
| | N-propylcarbamoyl; |
| for di-[(C1-C6)alkyl]carbamoyl: | N,N-dimethylcarbamoyl, N-ethyl- |
| | N-methylcarbamoyl and N,N-diethylcarbamoyl; |
| for (C3-C8)cycloalkyl: | cyclopropyl, cyclobutyl, cyclopentyl, cyclohexyl and |
| | cycloheptyl; |
| for (C3-C8)cycloalkyl(C1-C6)alkyl: | cyclopropylmethyl, cyclobutylmethyl, |
| | cyclopentylmethyl, cyclohexylmethyl and |
| | cycloheptylmethyl; |
| for (C3-C8)cycloalkyl(C1-C6)alkoxy: | cyclopropylmethoxy, cyclobutylmethoxy, |
| | cyclopentylmethoxy, cyclohexylmethoxy and |
| | cycloheptylmethoxy; |
| for (C3-C8)cycloalkylcarbonyl: | cyclopropylcarbonyl, cyclobutylcarbonyl, |
| | cyclopentylcarbonyl, cyclohexylcarbonyl |
| | andcycloheptylcarbonyl; |
| for (C3-C8)cycloalkyl(C1-C6)alkylcarbonyl: | cyclopropylmethylcarbonyl, |
| | cyclobutylmethylcarbonyl, |
| | cyclopentylmethylcarbonyl and |
| | cyclohexylmethylcarbonyl; |
| for (C3-C8)cycloalkylamino: | cyclopropylamino, cyclobutylamino, |
| | cyclopentylamino, cyclohexylamino and |
| | cycloheptylamino; |
| for (C3-C8)cycloalkylamino(C1-C6)alkyl: | cyclopropylaminomethyl, |
| | cyclopropylaminoethyl, cyclopropylaminopropyl, |
| | cyclobutylaminomethyl, cyclopentylaminoethyl, |
| | cyclopentylaminopropyl cyclohexylaminoethyl and |
| | cycloheptylaminoethyl; |
| for (C3-C8)cycloalkyl(C1-C6)alkylamino: | cyclopropylmethylamino, cyclopropylethylamino, |
| | cyclopentylmethylamino and |
| | cyclohexylmethylamino; |
| for (C3-C8)cycloalkyl(C1-C6)alkylamino(C1-C6)alkyl: | cyclopropylmethylaminomethyl, |
| | cyclopropylmethylaminoethyl, |
| | cyclopropylmethylaminopropyl, |
| | cyclopropylethylaminoethyl, |
| | cyclopropylethylaminobutyl, |
| | cyclopentylmethylaminoethyl, |
| | cyclopentylmethylaminobutyl and |
| | cyclohexylmethylaminoethyl; |
| for (C1-C6)alkoxyamino: | methoxyamino, ethoxyamino, propoxyamino and |
| | butoxyamino; |
| for (C1-C6)alkanoyl: | formyl, acetyl, propionyl, butyryl and isobuyryl; |
| for (C2-C6)alkanoylamino: | acetamido and propionamido; |
| for (C1-C6)alkylsulfonyl: | methylsulfonyl and ethylsulfonyl; and |
| for (C1-C6)alkylsulfinyl: | methylsulfinyl and ethylsulfinyl. |

Where the compounds according to the invention contain one or more asymmetrically substituted carbon atoms, the invention includes all stereoisomers, including enantiomers and diastereomers, and mixtures including racemic mixtures thereof.

Thus, it is to be understood that, insofar as certain of the compounds of formula (I) defined above may exist in optically active or racemic forms by virtue of one or more asymmetric carbon atoms, the invention includes in its definition any such optically active or racemic form which possesses the above-mentioned activity. In particular, the compound of formula (I) has a chiral centre on the azetidine ring (i.e. at the carbon atom attached to the group Q¹). The present invention encompasses all such stereoisomers having activity as herein defined, for example the (2R) and (2S) isomers (in particular the (2S) isomers). It is further to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ratio 50:50. The synthesis of optically active forms may be carried out by standard techniques of organic chemistry well known in the art, for example by synthesis from optically active starting materials or by resolution of a racemic form. Racemates may be separated into individual enantiomers using known procedures (cf. Advanced Organic Chemistry: 3rd Edition: author J March, p 104-107). A suitable procedure involves formation of diastereomeric derivatives by reaction of the racemic material with a chiral auxiliary, followed by separation, for example by chromatography, of the diastereomers and then cleavage of the auxiliary species. Similarly, the above-mentioned activity may be evaluated using the standard laboratory techniques referred to hereinafter.

It is also to be understood that, insofar as certain of the compounds of formula (I) defined above may exist in tautomeric forms, the invention includes in its definition any such tautomeric form which possesses the above-mentioned activity. Thus, the invention relates to all tautomeric forms of the compounds of formula (I) which inhibit IGF-1R tyrosine kinase activity in a human or animal. For example, the compounds of the invention may exist in the following alternative tautomeric forms (I′) and (I″):

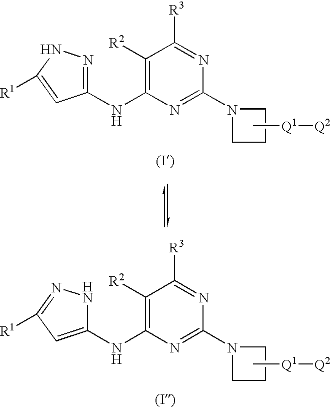

It is to be understood that certain compounds of formula (I) may exist in solvated as well as unsolvated forms such as, for example, hydrated forms. It is to be understood that the invention encompasses all such solvated forms which inhibit IGF-1R tyrosine kinase activity in a human or animal.

It is also to be understood that certain compounds of formula (I) may exhibit polymorphism, and that the invention encompasses all such forms which inhibit IGF-1R tyrosine kinase activity in a human or animal.

The compounds according to the invention may be provided as pharmaceutically-acceptable salts. Suitable pharmaceutically-acceptable salts include base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. In another aspect, where the compound is sufficiently basic, suitable salts include acid addition salts such as methanesulfonate, fumarate, hydrochloride, hydrobromide, citrate, maleate and salts formed with phosphoric and sulfuric acid.

In one aspect of the invention, a suitable value for R¹ is a (C3-C8)cycloalkyl(C1-C6)alkyl group (such as cyclopropylmethyl, cyclopentylmethyl or cyclohexylmethyl), which group is optionally substituted by one or more substituents selected from halogeno and (1-4C)alkoxy.

In another aspect of the invention, a suitable value for R¹ is a (C1-C6)alkyl group (for example a (C1-C4)alkyl group, such as methyl, ethyl, propyl, isopropyl or tert-butyl) or a (C3-C8)cycloalkyl group (for example a (C3-C6)cycloalkyl group, such as cyclopropyl, cyclopentyl or cyclohexyl), which group is optionally substituted by one or more substituents selected from halogeno and (1-4C)alkoxy. Another suitable value for R¹ is an unsubstituted (C1-C6)alkyl group (for example a (C1-C4)alkyl group) or an unsubstituted (C3-C8)cycloalkyl group (for example a (C3-C6)cycloalkyl group).

In another aspect of the invention, a suitable value for R¹ is an unsubstituted (C1-C4)alkyl group. For example, R¹ may be methyl, ethyl or tert-butyl, especially methyl or tert-butyl, more especially methyl.

In another aspect of the invention, a suitable value for R¹ is a (C3-C6)cycloalkyl group, such as cyclopropyl.

In one aspect of the invention, a suitable value for R² is hydrogen or trifluoromethyl.

In another aspect of the invention, a suitable value for R² is halogeno (such as fluoro, chloro, bromo or iodo, especially chloro or fluoro, more especially chloro).

In another aspect of the invention, a suitable value for R² is hydrogen.

In one aspect of the invention, R³ is selected from hydrogen, hydroxy or halogeno, or from a (C1-C6)alkyl, (C2-C6)alkenyl, (C2-C6)alkynyl, (C3-C8)cycloalkyl, (C3-C8)cycloalkyl(C1-C6)alkyl, (C1-C6)alkoxy, (C3-C8)cycloalkyl(C1-C6)alkoxy, (C1-C6)alkylcarbonyl, (C1-C6)alkoxycarbonyl, amino, (C1-C6)alkylamino, di-[(C1-C6)alkyl]amino, (C3-C8)cycloalkylamino, (C3-C8)cycloalkyl(C1-C6)alkylamino, (C1-C6)alkoxyamino, carbamoyl, (C1-C6)alkylcarbamoyl, di-[(C1-C6)alkyl]carbamoyl, —C(O)R^{3b}, —OR^{3b}, —NHR^{3b}, —N[(C1-C6)alkyl]R^{3b}, —S(O)ₘR^{3a} or —N(R^{3c})C(O)R^{3a} group, wherein R^{3a} is selected from a (C1-C6)alkyl or (C1-C6)alkoxy group, m is 0, 1 or 2, R^{3b} is a saturated monocyclic 4-, 5- or 6-membered heterocyclic ring comprising at least one ring heteroatom selected from nitrogen, oxygen and sulfur and R^{3c} is selected from hydrogen and (C1-C6)alkyl, or R³ is a saturated monocyclic 5- or 6-membered heterocyclic ring comprising at least one ring heteroatom selected from nitrogen, oxygen and sulfur, or R³ is a 5- or 6-membered heteroaromatic ring comprising at least one ring heteroatom selected from nitrogen, oxygen and sulfur, or R³ is a 2,7-diazaspiro[3.5]nonane group. Each of these groups or rings within R³ may be optionally substituted by one or more (for example one or two, particularly one) substituents independently selected from (C1-C6)alkyl, (C1-C6)alkoxy, (C1-C6)alkoxy(C1-C6)alkyl, (C1-C6)alkoxy(C1-C6)alkoxy, halogeno, hydroxy, trifluoromethyl, tri-[(C1-C4)alkyl]silyl, cyano, amino, (C1-C6)alkylamino, di-[(C1-C6)alkyl]amino, amino(C1-C6)alkyl, (C1-C6)alkylamino(C1-C6)alkyl, di-[(C1-C6)alkyl]amino(C1-C6)alkyl, (C1-C6)alkoxycarbonyl, carbamoyl, (C1-C6)alkylcarbamoyl, di-[(C1-C6)alkyl]carbamoyl, (C1-C6)alkylthio, (C1-C6)alkylsulfonyl, (C1-C6)alkylsulfinyl, (C1-C6)alkanoyl, an alkanoylamino group —N(R^{3d})C(O)R^{3e} wherein R^{3d} is selected from hydrogen and (C1-C6)alkyl and R^{3e}, is selected from a (C1-C6)alkyl, (C3-C8)cycloalkyl, (C3-C8)cycloalkyl(C1-C6)alkyl or (C1-C6)alkoxy group, or a saturated monocyclic 3-, 4-, 5-, 6- or 7-membered ring, which ring may optionally comprise one or more heteroatoms selected from nitrogen, oxygen and sulfur, any of which substituents may be optionally substituted by one or more (for example one or two, particularly one) (C1-C4)alkyl, hydroxy or cyano groups. Any saturated monocyclic ring within R³ optionally bears 1 or 2 oxo or thioxo substituents.

In another aspect of the invention, R³ is selected from hydrogen, hydroxy or halogeno, or from a (C1-C6)alkyl, (C2-C6)alkenyl, (C2-C6)alkynyl, (C3-C8)cycloalkyl, (C1-C6)alkoxy, (C1-C6)alkoxycarbonyl, amino, (C1-C6)alkylamino, di-[(C1-C6)alkyl]amino, (C3-C8)cycloalkylamino, carbamoyl, (C1-C6)alkylcarbamoyl, di-[(C1-C6)alkyl]carbamoyl, —C(O)R^{3b}, OR^{3b}, —NHR^{3b} or —S(O)ₘR^{3a} group, wherein R^{3a} is a (C1-C6)alkyl group, m is 0 and R^{3b} is a saturated monocyclic 4-, 5- or 6-membered heterocyclic ring comprising at least one ring heteroatom selected from nitrogen, oxygen and sulfur, or R³ is a saturated monocyclic 5- or 6-membered heterocyclic ring comprising at least one ring heteroatom selected from nitrogen and oxygen, or R³ is a 5- or 6-membered heteroaromatic ring comprising at least one ring heteroatom selected from nitrogen and oxygen. Each of these groups or rings within R³ may be optionally substituted by one or more (for example one or two, particularly one) substituents independently selected from (C1-C6)alkyl, (C1-C6)alkoxy, (C1-C6)alkoxy(C1-C6)alkyl, (C1-C6)alkoxy(C1-C6)alkoxy, halogeno, hydroxy, trifluoromethyl, tri-[(C1-C4)alkyl]silyl, amino, (C1-C6)alkylamino, di-[(C1-C6)alkyl]amino, amino(C1-C6)alkyl, (C1-C6)alkoxycarbonyl, carbamoyl, (C1-C6)alkylcarbamoyl, (C1-C6)alkylthio, (C1-C6)alkylsulfonyl, (C1-C6)alkanoyl, an alkanoylamino group —N(R^{3d})C(O)R^{3e} wherein R^{3d} is selected from hydrogen and (C1-C6)alkyl and R^{3e} is selected from a (C1-C6)alkyl or (C1-C6)alkoxy group, or a saturated monocyclic 3-, 4-, 5- or 6-membered ring, which ring may optionally comprise one or more heteroatoms selected from nitrogen, oxygen and sulfur, any of which substituents may be optionally substituted by one or more (for example one or two, particularly one) (C1-C4)alkyl, hydroxy or cyano groups. Any saturated monocyclic ring within R³ optionally bears 1 or 2 oxo substituents.

In another aspect of the invention, R³ is selected from hydrogen, hydroxy or halogeno, or from a (C1-C4)alkyl, (C2-C4)alkenyl, (C2-C4)alkynyl, (C1-C3)alkoxy, amino, (C1-C3)alkylamino, di-[(C1-C3)alkyl]amino, (C3-C6)cycloalkylamino, carbamoyl, (C1-C3)alkylcarbamoyl, di-[(C1-C3)alkyl]carbamoyl, —C(O)R^{3b}, R^{3b}, NHR^{3b} or S(O)ₘR^{3a} group, wherein R^{3a} is a (C1-C3)alkyl group, m is 0 and R^{3b} is a saturated monocyclic 4-, 5- or 6-membered heterocyclic ring comprising at least one ring heteroatom selected from nitrogen, oxygen and sulfur, or R³ is a saturated monocyclic 5- or 6-membered heterocyclic ring comprising at least one ring heteroatom selected from nitrogen and oxygen, or R³ is a 5- or 6-membered heteroaromatic ring comprising at least one ring heteroatom selected from nitrogen and oxygen. Each of these groups or rings within R³ may be optionally substituted by one or more substituents as defined above, in particular by one or more (for example one or two, particularly one) substituents independently selected from (C1-C3)alkyl, (C1-C3)alkoxy, (C1-C3)alkoxy(C1-C3)alkyl, (C1-C3)alkoxy(C1-C3)alkoxy, halogeno, hydroxy, trifluoromethyl, amino, (C1-C3)alkylamino, di-[(C1-C3)alkyl]amino, amino(C1-C3)alkyl, carbamoyl, (C1-C3)alkylcarbamoyl, (C1-C3)alkylthio, (C1-C3)alkylsulfonyl, (C1-C3)alkanoyl, an alkanoylamino group —N(R^{3d})C(O)R^{3e} wherein R^{3d} is selected from hydrogen and (C1-C3)alkyl and R^{3e} is selected from a (C1-C3)alkyl or (C1-C3)alkoxy group, or a saturated monocyclic 3-, 4-, 5- or 6-membered ring, which ring may optionally comprise one or more heteroatoms selected from nitrogen, oxygen and sulfur, any of which substituents may be optionally substituted by one or more (for example one or two, particularly one) (C1-C2)alkyl, hydroxy or cyano groups. Any saturated monocyclic ring within R³ optionally bears 1 oxo substituent.

In one aspect of the invention, R³ when it is substituted, may be substituted by one or more (for example, one, two or three, particularly one or two, more particularly one) substituents independently selected from (C1-C6)alkoxy (such as methoxy or ethoxy), (C1-C6)alkoxy(C1-C6)alkoxy (such as methoxyethoxy) or a saturated monocyclic 3-, 4-, 5-, 6- or 7-membered (for example 4-, 5-, 6- or 7-membered) ring, which ring may optionally comprise one or more heteroatoms selected from nitrogen, oxygen and sulfur (such as cyclopentyl, cyclohexyl, pylrolidinyl, piperidinyl, dioxanyl, morpholinyl, tetrahydrofuranyl or piperazinyl).

In another aspect of the invention, R³, when it is substituted, may be substituted by one or more (for example, one or two, particularly one) substituents independently selected from (C1-C6)alkyl, (C1-C6)alkoxy, halogeno, hydroxy, trifluoromethyl, amino, (C1-C6)alkylamino and di-[(C1-C6)alkyl]amino, or a saturated monocyclic 3-, 4-, 5-, 6- or 7-membered (for example 4-, 5-, 6- or 7-membered) ring, which ring may optionally comprise one or more heteroatoms selected from nitrogen, oxygen and sulfur.

In another aspect of the invention, when R³ carries a substituent that is a saturated monocyclic 3-, 4-, 5-, 6- or 7-membered (for example 4-, 5-, 6- or 7-membered) ring, which ring may optionally comprise one or more heteroatoms selected from nitrogen, oxygen and sulfur, that ring preferably comprises nitrogen and, optionally, one or two additional heteroatoms selected from nitrogen, oxygen and sulfur. For example, the saturated monocyclic 3-, 4-, 5-, 6- or 7-membered ring substituent on R³ may be pyrrolidine.

In another aspect of the invention, R³ is selected from hydrogen or from a (C1-C4)alkyl, (C1-C3)alkoxy or (C3-C5)cycloalkyl group, or R³ is a saturated monocyclic 5- or 6-membered heterocyclic ring comprising at least one ring heteroatom selected from nitrogen and oxygen. Each of these groups or rings within R³ may be optionally substituted by one or more (for example one or two, particularly one) substituents as defined above, in particular by one or more substituents independently selected from hydroxy and (C1-C3)alkoxy (especially (C1-C2)alkoxy, such as methoxy).

In another aspect of the invention, R³ is selected from hydrogen and halogeno, or from a (C1-C4)alkyl or (C1-C3)alkoxy group, or R³ is a saturated monocyclic 5- or 6-membered heterocyclic ring comprising at least one ring heteroatom selected from nitrogen and oxygen. Each of these groups or rings within R³ may be optionally substituted by one or more (for example one or two, particularly one) substituents as defined above, in particular by one or more substituents independently selected from hydroxy and (C1-C3)alkoxy (especially (C1-C2)alkoxy, such as methoxy).

In yet another aspect of the invention, R³ is selected from halogeno, or from a (C1-C4)alkyl or (C1-C3)alkoxy group, or R³ is a saturated monocyclic 5- or 6-membered heterocyclic ring comprising at least one ring heteroatom selected from nitrogen and oxygen. Each of these groups or rings within R³ may be optionally substituted by one or more (for example one or two, particularly one) substituents as defined above, in particular by one or more substituents independently selected from hydroxy and (C1-C3)alkoxy.

In another aspect of the invention, R³ is selected from hydrogen or halogeno, or from a (C1-C6)alkyl, (C2-C6)alkenyl, (C2-C6)alkynyl, (C1-C6)alkoxy, (C1-C6)alkylcarbonyl, (C1-C6)alkoxycarbonyl, amino, (C1-C6)alkylamino, di-[(C1-C6)alkyl]amino, carbamoyl, —C(O)R^{3b}, —OR^{3b}, —SR^{3b}, —NHR^{3b}, —N[(C1-C6)alkyl]R^{3b} or —S(O)ₘR^{3a} group (wherein m, R^{3a} and R^{3b} are as defined above), or R³ is a saturated monocyclic 5- or 6-membered heterocyclic ring comprising at least one ring heteroatom selected from nitrogen, oxygen and sulfur, each of which groups or rings may be optionally substituted by one or more (for example one or two, particularly one) substituents as defined hereinbefore.

In another aspect of the invention, R³ is selected from hydrogen or from a substituted or unsubstituted group selected from (C1-C6)alkyl (for example (C1-C4)alkyl, such as methyl, ethyl, propyl, isopropyl or tert-butyl), (C3-C8)cycloalkyl (for example(C3-C6)cycloalkyl, such as cyclopropyl, cyclopentyl or cyclohexyl), (C3-C8)cycloalkyl(C1-C6)alkyl (for example (C3-C6)cycloalkyl(C1-C4)alkyl, such as cyclopropylmethyl), (C1-C6)alkoxy (for example (C1-C4)alkoxy, such as methoxy, ethoxy, propoxy, isopropoxy and butoxy), (C1-C6)alkylcarbonyl (for example (C1-C4alkylcarbonyl, such as methylcarbonyl), (C3-C8)cycloalkylcarbonyl (for example (C3-C6)cycloalkylcarbonyl, such as cyclopropylcarbonyl), (C3-C8)cycloalkyl(C1-C6)alkylcarbonyl (for example (C3-C6)cycloalkyl(C1-C4)alkylcarbonyl, such as cyclopropylmethylcarbonyl), (C1-C6)alkoxycarbonyl (for example (C1-C4)alkoxycarbonyl, such as methoxycarbonyl), (C1-C6)alkylamino (for example (C1-C4)alkylamino, such as methylamino or ethylamino), (C3-C8)cycloalkylamino, (C3-C8)cycloalkyl(C1-C6)alkylamino, (C1-C6)alkoxyamino or —S(O)ₘR^{3a} (wherein m and R^{3a} are as defined above).

In another aspect of the invention, suitable values for R³ include, for example, hydrogen, hydroxy, chloro, fluoro or iodo, or a methyl, ethyl, n-propyl, iso-propyl, n-butyl, tert-butyl, ethenyl, propenyl, butenyl, pentenyl, ethynyl, propynyl, butynyl, methoxy, ethoxy, propoxy, tert-butoxy, cyclopropyl, cyclobutyl, methoxycarbonyl, ethoxycarbonyl, propoxycarbonyl, tert-butoxycarbonyl, methylamino, ethylamino, propylamino, dimethylamino, diethylamino, cyclobutylamino, cyclohexylamino, carbamoyl, N-methylcarbamoyl, N-ethylcarbamoyl, N-propylcarbamoyl, N-butylcarbamoyl, N,N-dimethylcarbamoyl, N-ethyl-N-methylcarbamoyl, pyrrolidinylcarbonyl, morpholinylcarbonyl, azetidinylcarbonyl, methylthio, ethylthio, piperidinylamino, tetrahydropyranylamino, tetrahydropyranyloxy, pyrrolidinyl, morpholinyl, piperazinyl, oxadiazolyl or 2,7-diazaspiro[3.5]nonan-7-yl group, each of which groups or rings may be optionally substituted by one or more (for example one or two, particularly one) substituents as defined above.

In yet another aspect of the invention, suitable values for R³ include, for example, hydrogen, hydroxy, chloro, fluoro, bromo, iodo, methyl, ethyl, propyl, iso-propyl, butyl, tert-butyl, cyclopropyl, cyclobutyl, cyclopentyl, cyclohexyl, trifluoromethyl, hydroxymethyl, methoxymethyl, ethoxymethyl, (2-methoxyethoxy)methyl, aminomethyl, methylaminomethyl, ethylaminomethyl, morpholinomethyl, piperazin-1-ylmethyl, 4-methylpiperazin-1-ylmethyl, pyrrolidin-1-ylmethyl, 2-hydroxyethyl, 2-methoxyethyl, 2-ethoxyethyl, 2-(ethoxycarbonyl)ethyl, 2-(N-methylcarbamoyl)ethyl, 3-hydroxypropyl, 3-methoxypropyl, 3-ethoxypropyl, 3-aminoprop-1-yl, 3-N,N-dimethylaminopropyl, 3-(tert-butoxycarbonylamino)prop-1-yl, 3-pyrrolidin-1-ylpropyl, ethenyl, propenyl, butenyl, pentenyl, 3-hydroxyprop-1-en-1-yl, 3-aminoprop-1-en-1-yl, 2-(methoxycarbonyl)ethen-1-yl, 3-(tert-butoxycarbonylamino)prop-1-en-1-yl, ethynyl, propynyl, butynyl, pentynyl, 3-hydroxyprop-1-yn-1-yl, 3-methoxyprop-1-yn-1-yl, 2-(trimetliylsilyl)ethynyl, 3-aminoprop-1-yn-1-yl, 3-methylaminoprop-1-yn-1-yl, 3-(dimethylamino)prop-1-yn-1-yl, 3-(N-methylacetamideo)prop-1-yn-1-yl, 3-acetamidoprop-1-yn-1-yl, methoxy, ethoxy, propoxy, butoxy, pentoxy, (5-oxopyrrolidin-2-yl)methoxy, tetrahydrofuran-3-ylmethoxy, 2-hydroxyethoxy, 2-ethoxyethoxy, 2-(2-hydroxyethoxy)ethoxy, 2-methoxyethoxy, (2-methoxyethoxy)ethoxy, 2-{N-[2-hydroxyethyl]-N-methyl-amino }ethoxy, 2-morpholinoethoxy, 2-(2-oxopyrrolidin-1-yl)ethoxy, 3-hydroxypropyloxy, 2-hydroxyprop-1-yloxy, 3-methoxyprop-1-yloxy, 2-methoxyprop-1-yloxy, 3-morpholinoprop-1-yloxy, 3-(methylthio)prop-1-yloxy, 3-(methylsulfonyl)propyl-1-oxy, methoxycarbonyl, tert-butoxycarbonyl, N-(tert-butoxycarbonyl)amino, methylamino, 2-methoxyethylamino, 2-aminoethylamino, 2-(dimethylamino)ethylamino, (N-2-metioxyethyl)-N-methylamino, 3-isopropoxyprop-1-ylamino, 2-(2-hydroxyethoxy)ethylamino, 2-(acetoamido)ethylamino, 2-(morpholin-4-yl)ethylamino, 2-methylprop-1-ylamino, 2-hydroxyprop-1-ylamino, 3-methoxypropylamino, 3-ethoxypropylamino, 2-isopropoxyethylamino, tetrahydrofuran-2-ylmethylamino, dimethylamino, N-(2-hydroxyethyl)-N-ethylamino, cyclopropylamino, cyclobutylamino, cyclopentylamino, 4-methylcyclohexylamino, 4-hydroxycyclohexylamino, carbamoyl, N-hydroxycarbamoyl, N-cyclopropylcarbamoyl, N-cyclopentylcarbamoyl, N-aminocarbamoyl, N-(acetylamino)carbamoyl, N-methylcarbamoyl, 2-hydroxyethylcarbamoyl, N-(2-hydroxypropyl)carbamoyl, N-(2,3-dihydroxypropyl)carbamoyl, N-(4-hydroxybutyl)carbamoyl, N-(2-methoxyethyl)carbamoyl, N-(2-(acetylamino)ethyl)carbamoyl, N-[2-(2-hydroxyethoxy)ethyl]carbamoyl, N-(carbamoylmethyl)carbamoyl, N-[2-(methylthio)ethyl]carbamoyl, N-(2-methoxyethyl)-N-methylcarbamoyl, pyrrolidin-1-ylcarbonyl, morpholinocarbonyl, azetidin-1-ylcarbonyl, (3-hydroxypyrrolidin-1-yl)carbonyl, methylthio, ethylthio, propylthio, 2,2,6,6-tetramethylpiperidin-4-ylamino, 4-tetrahydropyranylamino, tetrahydropyran-4-yloxy, pyrrolidin-1-yl, morpholino, piperazin-1-yl, 4-methylpiperazin-1-yl, 4-ethylpiperazin-1-yl, 4-isopropylpiperazin-1-yl, 4-(2-hydroxyethyl)piperazin-1-yl, 4-(3-hydroxypropyl)piperazin-1-yl, 4-(2-methoxyethyl)piperazin-1-yl, 4-(2-aminoethyl)piperazin-1-yl, 4-[2-(2-hydroxyethoxy)ethyl]piperazin-1-yl, 4-(2-cyanoethyl)piperazin-1-yl, 4-(tert-butoxycarbonyl)piperazin-1-yl, 1-formyl-piperazin-4-yl, 4-acetylpiperazin-1-yl, 4-(ethylsulfonyl)piperazin-1-yl, 4-aminopiperidin-1-yl, 4-(N-tert-butoxycarbonylamino)piperidin-1-yl, 3-hydroxypyrrolidin-1-yl, 3-dimethylamino-pyrrolidin-1-yl, cis-3,4-dihydroxypyrrolidin-1-yl, 5-methyl-[1,3,4]-oxadiazol-2-yl, 2,7-diazaspiro[3.5]nonan-7-yl and (tert-butoxycarbonyl)-2,7-diazaspiro[3.5]nonan-7-yl.

Further suitable values for R³ include, for example, hydrogen, hydroxy, chloro, iodo, methyl, ethyl, propyl, cyclopropyl, trifluoromethyl, hydroxymethyl, methoxymethyl, ethoxymethyl, (2-methoxyethoxy)methyl, aminomethyl, methylaminomethyl, morpholinomethyl, 4-methylpiperazin-1-ylmethyl, pyrrolidin-1-ylmethyl, 2-methoxyethyl, 2-(ethoxycarbonyl)ethyl, 2-(N-methylcarbamoyl)ethyl, 3-hydroxypropyl, 3-methoxypropyl, 3-aminoprop-1-yl, 3-N,N-dimethylaminopropyl, 3-(tert-butoxycarbonylamino)prop-1-yl, 3-pyrrolidin-1-ylpropyl, ethenyl, pent-3-en-1-yl, 3-hydroxyprop-1-en-1-yl, 3-aminoprop-1-en-1-yl, 2-(methoxycarbonyl)ethen-1-yl, 3-(tert-butoxycarbonylamino)prop-1-en-1-yl, ethynyl, 3-hydroxyprop-1-yn-1-yl, 3-methoxyprop-1-yn-1-yl, 2-(trimethylsilyl)ethynyl, 3-aminoprop-1-yn-1-yl, 3-methylaminoprop-1-yn-1-yl, 3-(dimethylamino)prop-1-yn-1-yl, 3-(N-methylacetamido)prop-1-yn-1-yl, 3-acetamidoprop-1-yn-1-yl, methoxy, ethoxy, (5-oxopyrrolidin-2-yl)methoxy (for example (2S)-(5-oxopyrrolidin-2-yl)methoxy or (2R)-(5-oxopyrrolidin-2-yl)methoxy), tetrahydrofuran-3-ylmethoxy, 2-hydroxyethoxy, 2-ethoxyethoxy, 2-(2-hydroxyethoxy)ethoxy, 2-methoxyethoxy, (2-methoxyethoxy)ethoxy, 2-{N-[2-hydroxyethyl]-N-methyl-amino }ethoxy, 2-morpholinoethoxy, 2-(2-oxopyrrolidin-1-yl)ethoxy, 2-(imidazolid-2-on-1-yl)ethoxy, 3-hydroxypropyloxy, 2-hydroxyprop-1-yloxy (for example (2R)-2-hydroxyprop-1-yloxy), 3-methoxyprop-1-yloxy, 2-methoxyprop-1-yloxy (for example (2S)-2-methoxyprop-1-yloxy), 3-morpholinoprop-1-yloxy, 3-(methylthio)prop-1-yloxy, 3-(methylsulfonyl)propyl-1-oxy, methoxycarbonyl, N-(tert-butoxycarbonyl)amino, methylamino, 2-methoxyethylamino, 2-aminoethylamino, 2-(dimethylamino)ethylamino, (N-2-methoxyethyl)-N-methylamino, 3-isopropoxyprop-1-ylamino, 2-(2-hydroxyethoxy)ethylamino, 2-(acetoamido)ethylamino, 2-(morpholin-4-yl)ethylamino, 2-methylprop-1-ylamino, 2-hydroxyprop-1-ylamino (for example (2R)-2-hydroxyprop-1-ylamino or (2S)-2-hydroxyprop-1-ylamino), 3-methoxypropylamino, 3-ethoxypropylamino, 2-isopropoxyethylamino, tetrahydrofuran-2-ylmethylamino (for example (2R)-tetrahydrofuran-2-ylmethylamino), dimethylamino, N-(2-hydroxyethyl)-N-ethylamino, cyclobutylamino, 4-methylcyclohexylamino, 4-hydroxycyclohexylamino, carbamoyl, N-hydroxycarbamoyl, N-cyclopropylcarbamoyl, N-cyclopentylcarbamoyl, N-aminocarbamoyl, N-(acetylamino)carbamoyl, N-methylcarbamoyl, 2-hydroxyethylcarbamoyl, N-(2-hydroxypropyl)carbamoyl (for example N-((R)-2-hydroxypropyl)carbamoyl), N-(2,3-dihydroxypropyl)carbamoyl (for example N-((2R)-2,3-dihydroxypropyl)carbamoyl), N-(4-hydroxybutyl)carbamoyl, N-(2-methoxyethyl)carbamoyl, N-(2-(acetylamino)ethyl)carbamoyl, N-[2-(2-hydroxyethoxy)ethyl]carbamoyl, N-(carbamoylmethyl)carbamoyl, N-[2-(methylthio)ethyl]carbamoyl, N-(2-methoxyethyl)-N-methylcarbamoyl, pyrrolidin-1-ylcarbonyl, morpholinocarbonyl, azetidin-1-ylcarbonyl, (3-hydroxypyrrolidin-1-yl)carbonyl (for example (3R)-3-hydroxypyrrolidin-1-ylcarbonyl), methylthio, 2,2,6,6-tetramethylpiperidin-4-ylamino, 4-tetrahydropyranylamino, tetrahydropyran-4-yloxy, pyrrolidin-1-yl, morpholino, piperazin-1-yl, 4-methylpiperazin-1-yl, 4-ethylpiperazin-1-yl, 4-isopropylpiperazin-1-yl, 4-(2-hydroxyethyl)piperazin-1-yl, 4-(3-hydroxypropyl)piperazin-1-yl, 4-(2-methoxyethyl)piperazin-1-yl, 4-(2-aminoethyl)piperazin-1-yl, 4-[2-(2-hydroxyethoxy)ethyl]piperazin-1-yl, 4-(2-cyanoethyl)piperazin-1-yl, 4-(tert-butoxycarbonyl)piperazin-1-yl, 1-formyl-piperazin-4-yl, 4-acetylpiperazin-1-yl, 4-(ethylsulfonyl)piperazin-1-yl, 4-aminopiperidin-1-yl, 4-(N-tert-butoxycarbonylamino)piperidin-1-yl, 3-hydroxypyrrolidin-1-yl (for example (3R)-3-hydroxypyrrolidin-1-yl), 3-dimethylamino-pyrrolidin-1-yl (for example (3R)-3-dimethylamino-pyrrolidin-1-yl), cis-3,4-dihydroxypyrrolidin-1-yl, 5-methyl-[1,3,4]-oxadiazol-2-yl, 2,7-diazaspiro[3.5]nonan-7-yl and (tert-butoxycarbonyl)-2,7-diazaspiro[3.5]nonan-7-yl.

Yet further suitable values for R³ include, for example, hydrogen, chloro, iodo, methyl, ethyl, trifluoromethyl, hydroxymethyl, methoxymethyl, ethoxymethyl, (2-methoxyethoxy)methyl, morpholinomethyl, 3-hydroxypropyl, 3-methoxypropyl, 3-N,N-dimethylaminopropyl, ethenyl, 3-hydroxyprop-1-en-1-yl, ethynyl, 3-hydroxyprop-1-yn-1-yl, 3-methoxyprop-1-yn-1-yl, 3-aminoprop-1-yn-1-yl, 3-methylaminoprop-1-yn-1-yl, 3-(dimethylamino)prop-1-yn-1-yl, 3-(N-methylacetamido)prop-1-yn-1-yl, 3-acetamidoprop-1-yn-1-yl, methoxy, ethoxy, (5-oxopyrrolidin-2-yl)methoxy (for example (2S)-(5-oxopyrrolidin-2-yl)methoxy or (2R)-(5-oxopyrrolidin-2-yl)methoxy), tetrahydrofuran-3-ylmethoxy, 2-hydroxyethoxy, 2-ethoxyethoxy, 2-(2-hydroxyethoxy)ethoxy, 2-methoxyethoxy, (2-methoxyethoxy)ethoxy, 2-{N-[2-hydroxyethyl]-N-methyl-amino}ethoxy, 2-morpholinoethoxy, 2-(2-oxopyrrolidin-1-yl)ethoxy, 2-(imidazolid-2-on-1-yl)ethoxy, 3-hydroxypropyloxy, 2-hydroxyprop-1-yloxy (for example (2R)-2-hydroxyprop-1-yloxy), 3-methoxyprop-1-yloxy, 2-methoxyprop-1-yloxy (for example (2S)-2-methoxyprop-1-yloxy), 3-morpholinoprop-1-yloxy, 3-(methylthio)prop-1-yloxy, 3-(methylsulfonyl)propyl-1-oxy, methylamino, 2-methoxyethylamino, 2-(methoxyethyl)amino, 2-(2-hydroxyethoxy)ethylamino, 2-(morpholin-4-yl)ethylamino, 2-methylprop-1-ylamino, 2-hydroxyprop-1-ylamino (for example (2R)-2-hydroxyprop-1-ylamino or (2S)-2-hydroxyprop-1-ylamino), 3-methoxypropylamino, 3-ethoxypropylamino, 2-isopropoxyethylamino, tetrahydrofuran-2-ylmethylamino (for example (2R)-tetrahydrofuran-2-ylmethylamino), dimethylamino, N-(2-hydroxyethyl)-N-ethylamino, cyclobutylamino, carbamoyl, N-cyclopropylcarbamoyl, N-methylcarbamoyl, 2-hydroxyethylcarbamoyl, N-(2-hydroxypropyl)carbamoyl (for example N-((R)-2-hydroxypropyl)carbamoyl), N-(2-methoxyethyl)carbamoyl, N-[2-(methylthio)ethyl]carbamoyl, pylolidin-1-ylcarbonyl, azetidin-1-ylcarbonyl, methylthio, 4-tetrahydropyranylamino, tetrahydropyran-4-yloxy, pyrrolidin-1-yl, morpholino, piperazin-1-yl, 4-methylpiperazin-1-yl, 4-ethylpiperazin-1-yl, 4-isopropylpiperazin-1-yl, 4-(2-hydroxyethyl)piperazin-1-yl, 4-(3-hydroxypropyl)piperazin-1-yl, 4-(2-methoxyethyl)piperazin-1-yl, 4-(2-cyanoethyl)piperazin-1-yl, , 4-acetylpiperazin-1-yl, 4-(ethylsulfonyl)piperazin-1-yl, 3-hydroxypyrrolidin-1-yl (for example (3R)-3-hydroxypyrrolidin-1-yl), 3-dimethylamino-pyrrolidin-1-yl (for example (3R)-3-dimethylamino-pyrrolidin-1-yl) and 1-formyl-piperazin-4-yl.

In another aspect of the invention, R³ is selected from hydrogen, chloro, methyl, ethyl, methoxy and morpholino.

In another aspect of the invention, R³ is selected from hydrogen, methyl, ethyl, methoxy and morpholino.

In yet another aspect of the invention, R³ is selected from chloro, methyl, methoxy and morpholino.

In yet another aspect of the invention, R³ is selected from methyl, methoxy and morpholino.

In yet another aspect of the invention, R³ is selected from hydrogen, chloro, methyl, ethyl, methoxy, ethoxy, 2-methoxyethoxy, 3-methoxyprop-1-yloxy and morpholino.

In yet another aspect of the invention, R³ is selected from hydrogen, methyl, ethyl, methoxy, ethoxy, 2-methoxyethoxy, 3-methoxyprop-1-yloxy and morpholino (especially methyl, ethyl, methoxy, ethoxy, 2-methoxyethoxy, 3-methoxyprop-1-yloxy and morpholino).

In one aspect of the invention, a suitable value for Q¹ is a 5- or 6-membered heteroaromatic ring comprising one, two, three or four ring heteroatoms, which may be the same or different, selected from nitrogen, oxygen and sulfur. For example, suitable values for Q¹ include thienyl, pyrazolyl, oxazolyl, isoxazolyl, thiadiazolyl, pyrrolyl, furanyl, thiazolyl, triazolyl, tetrazolyl, imidazolyl, pyrazinyl, pyridazinyl, pyrimidinyl and pyridyl.

In another aspect of the invention, a suitable value for Q¹ is a 5- or 6-membered heteroaromatic ring comprising one or two ring heteroatoms, which may be the same or different, selected from nitrogen and oxygen. For example, suitable values for Q¹ include pyridyl, pyrimidyl, pyrazinyl, pyridazinyl, imidazolyl, oxazolyl, tetrazolyl and isoxazolyl (particularly tetrazolyl and isoxazolyl).

In another aspect of the invention, a suitable value for Q¹ is a 5- or 6-membered heteroaromatic ring comprising a nitrogen and an oxygen ring heteroatom, for example an isoxazolyl ring (such as isoxazol-5-yl).

In yet another aspect of the invention, a suitable value for Q¹ is a 5- or 6-membered heteroaromatic ring comprising from one to four nitrogen ring heteroatoms. For example, suitable values for Q¹ include pyrrolyl, pyrazolyl, triazolyl, tetrazolyl, imidazolyl, pyrazinyl, pyridazinyl, pyrimidinyl and pyridyl.

The ring Q¹ may suitably be linked to the azetidine ring through any available ring atom, for example it may be linked via. a ring carbon or a ring nitrogen atom. In particular, Q¹ may be linked to the azetidine ring via. a ring carbon atom, for example via. a ring carbon atom that is adjacent to a heteroatom.

In addition to being substituted by Q², Q¹ is optionally substituted by at least one substituent (for example, one, two, three or four substituents), which may be the same or different, independently selected from (C1-C6)alkyl (such as methyl, ethyl, n-propyl, isopropyl, n-butyl, isobutyl, tert-butyl, n-pentyl or n-hexyl) and (C1-C6)alkoxy (such as methoxy, ethoxy, n-propoxy, n-butoxy, tert-butoxy, n-pentoxy or n-hexoxy) (either of which (C1-C6)alkyl and (C1-C6)alkoxy substituent groups may be optionally substituted by at least one substituent, for example one, two, three or four substituents, independently selected from halogeno (such as fluoro, chloro, bromo or iodo), amino, hydroxy and trifluoromethyl), halogeno (such as fluoro, chloro, bromo or iodo), nitro, cyano, —NR⁴R⁵, carboxy, hydroxy, (C2-C6)alkenyl (such as ethenyl), (C3-C8)cycloalkyl (such as cyclopropyl, cyclobutyl, cyclopentyl or cyclohexyl), (C3-C8)cycloalkyl(C1-C6)alkyl (such as cyclopropylmethyl), (C1-C4)alkoxycarbonyl (such as methoxycarbonyl or ethoxycarbonyl), (C1-C4)alkylcarbonyl (such as methylcarbonyl, ethylcarbonyl, n-propylcarbonyl, isopropylcarbonyl or n-butylcarbonyl), (C2-C6)alkanoylamino (such as acetamido or propionamido), phenylcarbonyl, —S(O)ₚ(C1-C4)alkyl (such as methylthio, ethylthio, methylsulfinyl, ethylsulfinyl, mesyl or ethylsulfonyl), —C(O)NR⁶R⁷ and —SO₂NR⁸R⁹ (where p, R⁴, R⁵, R⁶, R⁷, R⁸ and R⁹ are as defined above).

In one aspect of the invention, R⁴, R⁵, R⁶, R⁷, R⁸ and R⁹ may each suitably independently represent hydrogen or (C1-C4)alkyl (such as methyl, ethyl, propyl or butyl), or suitably R⁴ and R⁵, or R⁶ and R⁷, or R⁸ and R⁹, when taken together with the nitrogen atom to which they are attached, may each independently form a saturated heterocyclic ring such as pyrrolidinyl or piperidinyl.

In one aspect of the invention, Q¹ is substituted by Q2 and is optionally substituted by at least one substituent independently selected from (C1-C6)alkyl, (C1-C6)alkoxy, halogeno and (C3-C8)cycloalkyl.

In another aspect of the invention, Q¹ is substituted only by Q².

In one aspect of the invention, a suitable value for Q² is a substituted or unsubstituted (C1-C6)alkyl (such as methyl, ethyl, propyl or butyl), (C3-C8)cycloalkyl (such as cyclopropyl, cyclobutyl, cyclopentyl or cyclohexyl) or (C3-C8)cycloalkyl(C1-C6)alkyl (such as cyclopropylmethyl) group, or a saturated or unsaturated 5- or 6-membered monocyclic ring which may comprise at least one ring heteroatom (for example, one, two, three or four heteroatoms) selected from nitrogen, oxygen and sulfur (such as phenyl, pyridyl, imidazolyl, isoxazolyl, pyrazolyl, furyl, pyrazinyl, pyridazinyl, pyrimidinyl, pyrrolyl , thiazolyl, oxazolyl, isothiazolyl, triazolyl, tetrahydrofuranyl or thienyl, particularly pyridyl, pyrazinyl, thiazolyl, tetraliydrofuranyl or pyrimidinyl).

In another aspect of the invention, a suitable value for Q² is a substituted or unsubstituted (C1-C6)alkyl or (C3-C8)cycloalkyl group, or a substituted or unsubstituted saturated or unsaturated 5- or 6-membered monocyclic ring which may comprise at least one ring heteroatom selected from nitrogen, oxygen and sulfur. For example, suitable values for Q² include a substituted or unsubstituted group selected from methyl, cyclopropyl, pyridyl, pyrazinyl, thiazolyl, tetrahydrofuranyl or pyrimidinyl.

In yet another aspect of the invention, a suitable value for Q² is a substituted or unsubstituted (C1-C4)alkyl (such as methyl) or (C3-C6)cycloalkyl (such as cyclopropyl) group, or an optionally substituted unsaturated 5- or 6-membered monocyclic ring comprising one or two ring heteroatoms, which may be the same or different, selected from nitrogen, oxygen and sulfur, such as imidazolyl, isoxazolyl, pyrazolyl, furyl, pyrazinyl (especially pyrazin-2-yl), pyridazinyl, pyrimidinyl (especially pyrimidin-2-yl), pyrrolyl, oxazolyl, isothiazolyl, triazolyl, tetrahydrofuranyl or thienyl, especially pyridyl (preferably pyrid-2-yl or pyrid-3-yl) or thiazolyl (especially thiazol-2-yl or thiazol-4-yl) or tetrahydrofuranyl (especially tetrahydrofuran-3-yl).

In yet another aspect of the invention, a suitable value for Q² is an optionally substituted unsaturated 5- or 6-membered monocyclic ring comprising one or two ring heteroatoms, which may be the same or different, selected from nitrogen, oxygen and sulfur, such as pyrazinyl (especially pyrazin-2-yl), pyrimidinyl (especially pyrimidin-2-yl), pyridyl (especially pyrid-2-yl or pyrid-3-yl) or thiazolyl (especially thiazol-2-yl).

In yet another aspect of the invention, a suitable value for Q is an optionally substituted unsaturated 5- or 6-membered monocyclic ring comprising one or two ring nitrogen atoms independently selected from nitrogen, oxygen and sulfur, such as pyridyl (especially pyrid-2-yl or pyrid-3-yl, more especially pyrid-2-yl), pyrazinyl (especially pyrazin-2-yl) or pyrimidinyl (especially pyrimidin-2-yl). A particular value for Q² in this aspect of the invention is pyridyl (especially pyrid-2-yl or pyrid-3-yl, more especially pyrid-2-yl).

In yet another aspect of the invention, a suitable value for Q² is (optionally substituted) pyrazinyl (especially pyrazin-2-yl).

In yet another aspect of the invention, a suitable value for Q² is (optionally substituted) pyrimidinyl (especially pyrimidin-2-yl.

In yet another aspect of the invention, a suitable value for Q² is (optionally substituted) pyridyl (especially pyrid-2-yl or pyrid-3-yl).

In yet another aspect of the invention, a suitable value for Q² is (optionally substituted) thiazolyl (especially thiazol-2-yl).

In one aspect of the invention, suitable substituents for Q², when it is substituted, include one or more (for example, one, two, three or four) substituents independently selected from (C1-C6)alkyl and (C1-C6)alkoxy (either of which (C1-C6)alkyl and (C1-C6)alkoxy substituent groups may be optionally substituted by at least one substituent (for example, one, two, three or four substituents) independently selected from halogeno, amino, hydroxy and trifluoromethyl), halogeno, nitro, cyano, —NR¹⁰OR¹¹, carboxy, hydroxy, (C2-C6)alkenyl, (C3-C8)cycloalkyl, (C1-C6)alkoxycarbonyl, (C1-C6)alkylcarbonyl, (C2-C6)alkanoylamino, phenylcarbonyl, —S(O)ₙ(C1-C6)alkyl, —C(O)NR^{12 R}¹³ and —SO₂NR¹⁴R¹⁵ (where n, R¹⁰, R¹¹, R¹², R¹³, R¹⁴ and R¹⁵ are as defined above).

In another aspect of the invention, suitable substituents for Q², when it is substituted, include one or more (for example, one or two, particularly one) substituents independently selected from (C1-C4)alkyl, (C1-C4)alkoxy, cyano and —NR¹⁰R¹¹ (where R¹⁰ and R¹¹ are as defined above).

In yet another aspect of the invention, suitable substituents for Q², when it is substituted, include one or more (for example, one or two, particularly one) substituents independently selected from (C1-C4)alkyl (such as methyl or ethyl), (C1-C4)alkoxy (such as methoxy or ethoxy) and cyano.

Suitably, R¹⁰, R¹¹, R¹², R¹³, R¹⁴ and R¹⁵ may each independently represent hydrogen or (C1-C4)alkyl (such as methyl), or R¹⁰ and R¹¹, or R¹² and R¹³, or R¹⁴ and R¹⁵, when taken together with the nitrogen atom to which they are attached, may each suitably form a saturated heterocyclic ring, such as pyrrolidinyl or piperidinyl.

It will be appreciated that the number and nature of substituents on rings in the compounds of the invention will be selected so as to avoid sterically undesirable combinations.

In one group of compounds of formula (I) according to the invention, R¹ is a (C1-C4)alkyl group; R² is hydrogen; R³ is selected from hydrogen, halogeno, (C1-C4)alkyl, (C1-C4)alkoxy and a saturated monocyclic 5- or 6-membered heterocyclic ring comprising at least one ring heteroatom selected from nitrogen, oxygen and sulfur; Q¹ is a substituted 5- or 6-membered heteroaromatic ring comprising one or two ring heteroatoms, which may be the same or different, selected from nitrogen and oxygen; and Q² is an optionally substituted unsaturated 5- or 6-membered monocyclic ring comprising one or two ring heteroatoms, which may be the same or different, selected from nitrogen, oxygen and sulfur. For example, within this group, suitable values for Q¹ are isoxazolyl and tetrazolyl (especially isoxazolyl) and suitable values for Q² are methyl, cyclopropyl, tetrahydrofuranyl, pyrazinyl, thiazolyl, pyrimidinyl and pyridyl (especially pyridyl, pyrimidinyl and pyrazinyl, more especially pyridyl).

In another group of compounds of formula (I) according to the invention, R¹ is a (C1-C4)alkyl group; R² is hydrogen; R³ is selected from (C1-C4)alkyl, (C1-C4)alkoxy and a saturated monocyclic 5- or 6-membered heterocyclic ring comprising at least one ring heteroatom selected from nitrogen, oxygen and sulfur; Q¹ is a substituted 5-membered heteroaromatic ring comprising one or two ring heteroatoms, which may be the same or different, selected from nitrogen and oxygen; and Q² is an optionally substituted unsaturated 5- or 6-membered monocyclic ring comprising one or two ring heteroatoms, which may be the same or different, selected from nitrogen, oxygen and sulfur. For example, within this group, a suitable value for Q¹ is isoxazolyl and suitable values for Q² are pyrazinyl, thiazolyl, pyrimidinyl and pyridyl (especially pyridyl, pyrimidinyl and pyrazinyl, more especially pyridyl).

In one aspect of the invention, suitable values for the group of sub-formula (i) (which is attached to the 2-position of the pyrimidine ring of formula (I)):

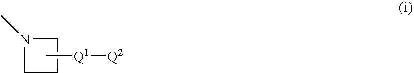

include, for example, 2-[3-(pyrid-2-yl)isoxazol-5-yl]azetidin-1-yl, 2-(3-methylisoxazol-5-yl]azetidin-1-yl, 2-[3-cyclopropylisoxazol-5-yl]azetidin-1-yl, 2-[3-(thiazol-2-yl)isoxazol-5-yl]azetidin-1-yl, 2-[3-(thiazol-4-yl)isoxazol-5-yl]azetidin-1-yl, 2-[3-(pyrid-3-yl)isoxazol-5-yl]azetidin-1-yl, 2-(3-{tetrahydrofuran-3-yl}isoxazol-5-yl]azetidin-1-yl, 2-[3-(2-methoxypyrid-3-yl)isoxazol-5-yl]azetidin-1-yl, 2-(2-methyl-2H-tetrazol-5-yl)azetidin-1-yl-, 2-[3-(pyrazin-2-yl)isoxazol-5-yl]azetidin-1-yl, 2-{3-(pyrimid-2-yl)isoxazol-5-yl}azetidin-1-yl, 2-{3-(3-methoxypyrazin-2-yl)isoxazol-5-yl}azetidin-1-yl, 2-{3-(3-hydroxypyrazin-2-yl)isoxazol-5-yl}azetidin-1-yl, 2-[3-(2-methylpyrid-3-yl)isoxazol-5-yl]azetidin-1-yl, 2-[3-(2-cyanopyrid-3-yl)isoxazol-5-yl]azetidin-1-yl, 2-[3-(2-methylaminopyrid-3-yl)isoxazol-5-yl]azetidin-1-yl and 2-{3-(3-methylpyrazin-2-yl)isoxazol-5-yl}azetidin-1-yl (where, for the avoidance of any doubt, it is the azetidin-1-yl group that is attached to the 2-position of the pyrimidine ring in formula (I)).

In another aspect of the invention, suitable values for the group of sub-formula (i) (which is attached to the 2-position of the pyrimidine ring of formula (I)) include, for example, 2-[3-(pyrid-2-yl)isoxazol-5-yl]azetidin-1-yl, 2-{3-(pyrimid-2-yl)isoxazol-5-yl}azetidin-1-yl, 2-{3-(3-methoxypyrazin-2-yl)isoxazol-5-yl}azetidin-1-yl, 2-[3-(2-methylpyrid-3-yl)isoxazol-5-yl]azetidin-1-yl, 2-[3-(2-cyanopyrid-3-yl)isoxazol-5-yl]azetidin-1-yl, 2-[3-(2-methylaminopyrid-3-yl)isoxazol-5-yl]azetidin-1-yl and 2-{3-(3-methylpyrazin-2-yl)isoxazol-5-yl}azetidin-1-yl (where, for the avoidance of any doubt, it is the azetidin-1-yl group that is attached to the 2-position of the pyrimidine ring in formula (I)). In yet another aspect of the invention, a suitable value for the group of sub-formula (i) is, for example, 2-[3-(pyrid-2-yl)isoxazol-5-yl]azetidin-1-yl.

In yet another aspect of the invention, suitable values for the group of sub-formula (i) (which is attached to the 2-position of the pyrimidine ring of formula (I)) include, for example, 2-[3-(pyrid-2-yl)isoxazol-5-yl]azetidin-1-yl, 2-(3-methylisoxazol-5-yl]azetidin-1-yl, 2-[3-cyclopropylisoxazol-5-yl]azetidin-1-yl, 2-[3-(thiazol-2-yl)isoxazol-5-yl]azetidin-1-yl, 2-[3-(thiazol-4-yl)isoxazol-5-yl]azetidin-1-yl, 2-[3-(pyrid-3-yl)isoxazol-5-yl]azetidin-1-yl, 2-(3-{tetrahydrofuran-3-yl}isoxazol-5-yl]azetidin-1-yl, 2-[3-(2-methoxypyrid-3-yl)isoxazol-5-yl]azetidin-1-yl, 2-(2-methyl-2H-tetrazol-5-yl)azetidin-1-yl, 2-[3-(pyrazin-2-yl)isoxazol-5-yl]azetidin-1-yl, 2-{3-(pyrimid-2-yl)isoxazol-5-yl}azetidin-1-yl, 2-{3-(3-methoxypyrazin-2-yl)isoxazol-5-yl}azetidin-1-yl, 2-{3-(3-ethoxypyrazin-2-yl)isoxazol-5-yl}azetidin-1-yl, 2-{3-(3-hydroxypyrazin-2-yl)isoxazol-5-yl}azetidin-1-yl, 2-[3-(2-methylpyrid-3-yl)isoxazol-5-yl]azetidin-1-yl, 2-[3-(2-cyanopyrid-3-yl)isoxazol-5-yl]azetidin-1-yl, 2-[3-(2-[3-(2-methylaminopyrid-3-yl)isoxazol-5-yl]azetidin-1-yl, 2-{3-(3-ethylpyrazin-2-yl)isoxazol-5-yl}azetidin-1-yl and 2-{3-(3-methylpyrazin-2-yl)isoxazol-5-yl}azetidin-1-yl (where, for avoidance of any doubt, it is the azetidin-1-yl group that is attached to the 2-position of the pyrimidine ring in formula (I)).

In another aspect of the invention, suitable values for the group of sub-formula (i) (which is attached to the 2-position of the pyrimidine ring of formula (I)) include, for example, 2-[3-(pyrid-2-yl)isoxazol-5-yl]azetidin-1-yl, 2-{3-(pyrimid-2-yl)isoxazol-5-yl}azetidin-1-yl, 2-{3-(3-methoxypyrazin-2-yl)isoxazol-5-yl}azetidin-1-yl, 2-{3-(3-ethoxypyrazin-2-yl)isoxazol-5-yl}azetidin-1-yl, 2-[3-(2-methylpyrid-3-yl)isoxazol-5-yl]azetidin-1-yl, 2-[3-(2-cyanopyrid-3-yl)isoxazol-5-yl]azetidin-1-yl, 2-[3-(2-methylaminopyrid-3-yl)isoxazol-5-yl]azetidin-1-yl, 2-{3-(3-ethylpyrazin-2-yl)isoxazol-5-yl}azetidin-1-yl and 2-{3-(3-methylpyrazin-2-yl)isoxazol-5-yl}azetidin-1-yl (where, for the avoidance of any doubt, it is the azetidin-1-yl group that is attached to the 2-position of the pyrimidine ring in formula (I)).

In another aspect of the invention, suitable values for the group of sub-formula (i) (which is attached to the 2-position of the pyrimidine ring of formula (I)) include, for example, 2-[3-(pyrid-2-yl)isoxazol-5-yl]azetidin-1-yl, 2-{3-(pyrimid-2-yl)isoxazol-5-yl}azetidin-1-yl, 2-{3-(3-methoxypyrazin-2-yl)isoxazol-5-yl}azetidin-1-yl, 2-{3-(3-ethoxypyrain-2-yl)isoxazol-5-yl}azetidin-1-yl, 2-[3-(2-methylpyrid-3-yl)isoxazol-5-yl]azetidin-1-yl, 2-[3-(2-cyanopyrid-3-yl)isoxazol-5-yl]azetidin-1-yl, 2-[3-(thiazol-2-yl)isoxazol-5-yl]azetidin-1-yl, 2-{3-(3-ethylpyrazin-2-yl)isoxazol-5-yl}azetidin-1-yl and 2-{3-(3-methylpyrazin-2-yl)isoxazol-5-yl}azetidin-1-yl (where, for the avoidance of any doubt, it is the azetidin-1-yl group that is attached to the 2-position of the pyrimidine ring in formula (I)).

A particular embodiment of the present invention is a compound of formula (Ia):

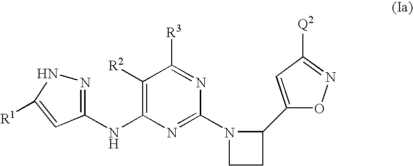

wherein

R¹ is selected from a (C1-C6)alkyl, (C3-C8)cycloalkyl or (C3-C8)cycloalkyl(C1-C6)alkyl group, each of which groups may be optionally substituted by one or more substituents independently selected from halogeno and (C1-C6)alkoxy;

R² is selected from hydrogen, halogeno and trifluoromethyl;

R³ is selected from hydrogen, hydroxy and halogeno, or from a (C1-C6)alkyl, (C2-C6)alkenyl, (C2-C6)alkynyl, (C3-C8)cycloalkyl, (C3-C8)cycloalkyl(C1-C6)alkyl, (C1-C6)alkoxy, (C3-C8)cycloalkyl(C1-C6)alkoxy, (C1-C6)alkylcarbonyl, (C3-C8)cycloalkylcarbonyl, (C3-C8)cycloalkyl(C1-C6)alkylcarbonyl, (C1-C6)alkoxycarbonyl, amino, (C1-C6)alkylamino, di-[(C1-C6)alkyl]amino, (C3-C8)cycloalkylamino, (C3-C8)cycloalkyl(C1-C6)alkylamino, (C1-C6)alkoxyamino, carbamoyl, (C1-C6)alkylcarbamoyl, di-[(C1-C6)alkyl]carbamoyl, —C(O)R^{3b}, —OR^{3b}, —SR , —NHR^{3b}, —N[(C1-C6)alkyl]R^{3b}, —S(O)ₘR^{3a} or —N(R^{3c})C(O)R^{3a} group, wherein R^{3a} is selected from a (C1-C6)alkyl, (C3-C8)cycloalkyl, (C3-C8)cycloalkyl(C1-C6)alkyl or (C1-C6)alkoxy group, m is 0, 1 or 2, R^{3b} is a saturated monocyclic 4-, 5- or 6-membered heterocyclic ring comprising at least one ring heteroatom selected from nitrogen, oxygen and sulfur and R^{3c} is selected from hydrogen and (C1-C6)alkyl, or R³ is a saturated monocyclic 5- or 6-membered heterocyclic ring comprising at least one ring heteroatom selected from nitrogen, oxygen and sulfur,

or R³ is a 5- or 6-membered heteroaromatic ring comprising at least one ring heteroatom selected from nitrogen, oxygen and sulfur,

or R³ is a 2,7-diazaspiro[3.5]nonane group,

each of which groups or rings within R³ may be optionally substituted by one or more substituents independently selected from (C1-C6)alkyl, (C1-C6)alkoxy, (C1-C6)alkoxy(C1-C6)alkyl, (C1-C6)alkoxy(C1-C6)alkoxy, halogeno, hydroxy, trifluoromethyl, tri-[(C1-C4)alkyl]silyl, cyano, amino, (C1-C6)alkylamino, di-[(C1-C6)alkyl]amino, (C3-C8)cycloalkylamino, (C3-C6)cycloalkyl(C1-C3)alkylamino, amino(C1-C6)alkyl, (C1-C6)alkylamino(C1-C6)alkyl, di-[(C1-C6)alkyl]amino(C1-C6)alkyl, (C3-C8)cycloalkylamino(C1-C6)alkyl, (C3-C6)cycloalkyl(C l-C3)alkylamino(C1-C6)alkyl, (C1-C6)alkoxycarbonyl, carbamoyl, (C1-C6)alkylcarbamoyl, di-[(C1-C6)alkyl]carbamoyl, (C1-C6)alkylthio, (C1-C6)alkylsulfonyl, (C1-C6)alkylsulfinyl, (C1-C6)alkanoyl, an alkanoylamino group —N(R^{3d})C(O)R^{3e} wherein R^{3d} is selected from hydrogen and (C1-C6)alkyl and R^{3e} is selected from a (C1-C6)alkyl, (C3-C8)cycloalkyl, (C3-C8)cycloalkyl(C1-C6)alkyl or (C1-C6)alkoxy group, or a saturated monocyclic 3-, 4-, 5-, 6- or 7-membered ring, which ring may optionally comprise one or more heteroatoms selected from nitrogen, oxygen and sulfur, any of which substituents may be optionally substituted by one or more (C1-C4)alkyl, hydroxy or cyano groups;

Q² is selected from a (C1-C6)alkyl, (C3-C6)cycloalkyl or (C3-C6)cycloalkyl(C1-C6)alkyl group or a saturated or unsaturated 5- or 6-membered monocyclic ring which may comprise at least one ring heteroatom selected from nitrogen, oxygen and sulfur, and wherein Q² is optionally substituted by one or more substituents independently selected from (C1-C6)alkyl and (C1-C6)alkoxy (either of which (C1-C6)alkyl and (C1-C6)alkoxy substituent groups may be optionally substituted by one or more substituents independently selected from halogeno, amino, hydroxy and trifluoromethyl), halogeno, nitro, cyano, —NR¹⁰R¹¹, carboxy, hydroxy, (C2-C6)alkenyl, (C3-C8)cycloalkyl, (C1-C6)alkoxycarbonyl, (C1-C6)alkylcarbonyl, (C2-C6)alkanoylamino, phenylcarbonyl, —S(O)ₙ(C1-C6)alkyl, —C(O)NR¹²R¹³ and —SO₂NR¹⁴R¹⁵, wherein R¹⁰, R¹¹, R¹², R¹³, R¹⁴ and R¹⁵ are each independently selected from hydrogen and (C1-C6)alkyl, or R¹⁰ and R¹¹, or R¹² and R¹³, or R¹⁴ and R¹⁵, when taken together with the nitrogen atom to which they are attached, may each independently form a saturated heterocyclic ring and n is 0, 1 or 2;

and wherein any saturated monocyclic ring optionally bears 1 or 2 oxo or thioxo substituents;

or a pharmaceutically-acceptable salt thereof.

In the compounds of formula (Ia), a suitable value for Q² is an optionally substituted unsaturated 5- or 6-membered monocyclic ring comprising one or two ring nitrogen atoms, such as pyridyl, pyrimidinyl or pyrazinyl, especially pyridyl (such as pyrid-2-yl).

In one aspect, particular compounds of the invention include, for example, any one or more compounds of formula (I) selected from:
6-chloro-2-{2-[3-(pyrid-2-yl)isoxazol-5-yl]azetidin-1-yl}-4-(5-methyl-1H-pyrazol-3-ylamino)pyrimidine;
6-morpholino-2-{2-[3-(pyrid-2-yl)isoxazol-5-yl]azetidin-1-yl}-4-(5-methyl-1H-pyrazol-3-ylamino)pyrimidine;
6-methoxy-2-{2-[3-(pyrid-2-yl)isoxazol-5-yl]azetidin-1-yl}-4-(5-methyl-1H-pyrazol-3-ylamino)pyrimidine;
6-methyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(3-methoxypyrazin-2-yl)isoxazol-5-yl]azetidin-1-yl}-4-(5-methyl-1H-pyrazol-3-ylamino)pyrimidine;
S-6-ethyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(2-methylpyrid-3-yl)isoxazol-5-yl]azetidin-1-yl}-4-(5-methyl-1H-pyrazol-3-ylamino)pyrimidine;
S-6-ethyl-2-{2-[3-(2-cyanopyrid-3-yl)isoxazol-5-yl]azetidin-1-yl}-4-(5-methyl-1H-pyrazol-3-ylamino)pyrimidine;
S-6-ethyl-2-{2-[3-(2-methylaminopyrid-3-yl)isoxazol-5-yl]azetidin-1-yl}-4-(5-methyl-1H-pyrazol-3-ylamino)pyrimidine;
S-6-methyl-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(2-methylaminopyrid-3-yl)isoxazol-5-yl]azetidin-1-yl}-4-(5-methyl-1H-pyrazol-3-ylamino)pyrimidine;
S-6-morpholino-2-{2-[3-(2-methylpyrid-3-yl)isoxazol-5-yl]azetidin-1-yl}-4-(5-methyl-1H-pyrazol-3-ylamino)pyrimidine;
S-6-methoxy-2-{2-[3-(2-methylpyrid-3-yl)isoxazol-5-yl]azetidin-1-yl}-4-(5-methyl-1H-pyrazol-3-ylamino)pyrimidine;
S-6-methoxy-2-{2-[3-(2-methylaminopyrid-3-yl)isoxazol-5-yl]azetidin-1-yl}-4-(5-methyl-1H-pyrazol-3-ylamino)pyrimidine;
S-2-{2-[3-(3-methylpyrazin-2-yl)isoxazol-5-yl]azetidin-1-yl}-4-(5-tert-butyl-1H-pyrazol-3-ylamino)pyrimidine;
S-2-{2-[3-(pyrid-2-yl)isoxazol-5-yl]azetidin-1-yl}-4-(5-tert-butyl-1H-pyrazol-3-ylamino)pyrimidine;
S-2-{2-[3-(pyrimid-2-yl)isoxazol-5-yl]azetidin-1-yl}-4-(5-tert-butyl-1H-pyrazol-3-ylamino)pyrimidine;
S-2-{2-[3-(2-methylpyrid-3-yl)isoxazol-5-yl]azetidin-1-yl}-4-(5-tert-butyl-1H-pyrazol-3-ylamino)pyrimidine; S-2-{2-[3-(2-cyanopyrid-3-yl)isoxazol-5-yl]azetidin-1-yl}-4-(5-tert-butyl-1H-pyrazol-3-ylamino)pyrimidine;
S-6-methyl-2-{2-[3-(3-methylpyrazin-2-yl)isoxazol-5-yl]azetidin-1-yl}-4-(5-tert-butyl-1H-pyrazol-3-ylamino)pyrimidine;
S-6-methyl-2-{2-[3-(pyrimid-2-yl)isoxazol-5-yl]azetidin-1-yl}-4-(5-tert-butyl-1H-pyrazol-3-ylamino)pyrimidine;
S-6-methyl-2-{2-[3-(2-cyanopyrid-3-yl)isoxazol-5-yl]azetidin-1-yl}-4-(5-tert-butyl-1H-pyrazol-3-ylamino)pyrimidine; and
S-6-methyl-2-{2-[3-(3-methoxypyrazin-2-yl)isoxazol-5-yl]azetidin-1-yl}-4-(5-tert-butyl-1H-pyrazol-3-ylamino)pyrimidine;
and pharmaceutically-acceptable salts thereof.

In another aspect, particular compounds of the invention include, for example, any one or more compounds of formula (I) selected from:
6-chloro-2-{2-[3-(pyrid-2-yl)isoxazol-5-yl]azetidin-1-yl}-4-(5-methyl-1H-pyrazol-3-ylamino)pyrimidine;
6-morpholino-2-{2-[3-(pyrid-2-yl)isoxazol-5-yl]azetidin-1-yl}-4-(5-methyl-1H-pyrazol-3-ylamino)pyrimidine;
6-methoxy-2-{2-[3-(pyrid-2-yl)isoxazol-5-yl]azetidin-1-yl}-4-(5-methyl-1H-pyrazol-3-ylamino)pyrimidine;
6-methyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(3-methoxypyrazin-2-yl)isoxazol-5-yl]azetidin-1-yl}-4-(5-methyl-1H-pyrazol-3-ylamino)pyrimidine;
S-6-ethyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(2-methylpyrid-3-yl)isoxazol-5-yl]azetidin-1-yl}-4-(5-methyl-1H-pyrazol-3-ylamino)pyrimidine;
S-6-ethyl-2-{2-[3-(2-cyanopyrid-3-yl)isoxazol-5-yl]azetidin-1-yl}-4-(5-methyl-1H-pyrazol-3-ylamino)pyrimidine;
S-6-methyl-2-{2-[3-(2-methylpyrid-3-yl)isoxazol-5-yl]azetidin-1-yl}-4-(5-methyl-1H-pyrazol-3-ylamino)pyrimidine;
S-6-morpholino-2-{2-[3-(2-methylpyrid-3-yl)isoxazol-5-yl]azetidin-1-yl}-4-(5-methyl-1H-pyrazol-3-ylamino)pyrimidine;
S-6-methoxy-2-{2-[3-(2-methylpyrid-3-yl)isoxazol-5-yl]azetidin-1-yl}-4-(5-methyl-1H-pyrazol-3-ylamino)pyrimidine;
S-2-{2-[3-(3-methylpyrazin-2-yl)isoxazol-5-yl]azetidin-1-yl}-4-(5-tert-butyl-1H-pyrazol-3-ylamino)pyrimidine;
S-2-{2-[3-(pyrid-2-yl)isoxazol-5-yl]azetidin-1-yl}-4-(5-tert-butyl-1H-pyrazol-3-ylamino)pyrimidine;
S-6-methyl-2-{2-[3-(3-methylpyrazin-2-yl)isoxazol-5-yl]azetidin-1-yl}-4-(5-tert-butyl-1H-pyrazol-3-ylamino)pyrimidine; and
S-6-methyl-2-{2-[3-(3-methoxypyrazin-2-yl)isoxazol-5-yl]azetidin-1-yl}-4-(5-tert-butyl-1H-pyrazol-3-ylamino)pyrimidine;
and pharmaceutically-acceptable salts thereof.

In another aspect, particular compounds of the invention include, for example, any one or more compounds of formula (I) selected from:
6-morpholino-2-{2-[3-(pyrid-2-yl)isoxazol-5-yl]azetidin-1-yl}-4-(5-methyl-1H-pyrazol-3-ylamino)pyrimidine;
6-methoxy-2-{2-[3-(pyrid-2-yl)isoxazol-5-yl]azetidin-1-yl}-4-(5-methyl-1H-pyrazol-3-ylamino)pyrimidine;
6-methyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(3-methoxypyrazin-2-yl)isoxazol-5-yl]azetidin-1-yl}-4-(5-methyl-1H-pyrazol-3-ylamino)pyrimidine;
S-6-ethyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(2-methylpyrid-3-yl)isoxazol-5-yl]azetidin-1-yl}-4-(5-methyl-1H-pyrazol-3-ylamino)pyrimidine;
S-6-ethyl-2-{2-[3-(2-cyanopyrid-3-yl)isoxazol-5-yl]azetidin-1-yl}-4-(5-methyl-1H-pyrazol-3-ylamino)pyrimidine;
S-6-methyl-2-{2-[3-(2-methylpyrid-3-yl)isoxazol-5-yl]azetidin-1-yl}-4-(5-methyl-1H-pyrazol-3-ylamino)pyrimidine;
S-6-morpholino-2-{2-[3-(2-methylpyrid-3-yl)isoxazol-5-yl]azetidin-1-yl}-4-(5-methyl-1H-pyrazol-3-ylamino)pyrimidine;
S-6-methoxy-2-{2-[3-(2-methylpyrid-3-yl)isoxazol-5-yl]azetidin-1-yl}-4-(5-methyl-1H-pyrazol-3-ylamino)pyrimidine;
S-2-{2-[3-(3-methylpyrazin-2-yl)isoxazol-5-yl]azetidin-1-yl}-4-(5-tert-butyl-1H-pyrazol-3-ylamino)pyrimidine;
S-2-{2-[3-(pyrid-2-yl)isoxazol-5-yl]azetidin-1-yl}-4-(5-tert-butyl-1H-pyrazol-3-ylamino)pyrimidine;
S-6-methyl-2-{2-[3-(3-methylpyrazin-2-yl)isoxazol-5-yl]azetidin-1-yl}-4-(5-tert-butyl-1H-pyrazol-3-ylamino)pyrimidine; and
S-6-methyl-2-{2-[3-(3-methoxypyrazin-2-yl)isoxazol-5-yl]azetidin-1-yl}-4-(5-tert-butyl-1H-pyrazol-3-ylamino)pyrimidine;
and pharmaceutically-acceptable salts thereof.

In another aspect, particular compounds of the invention include, for example, any one or more compounds of formula (I) selected from:
6-chloro-2-{2-[3-(pyrid-2-yl)isoxazol-5-yl]azetidin-1-yl}-4-(5-methyl-1H-pyrazol-3-ylamino)pyrimidine;
6-morpholino-2-{2-[3-(pyrid-2-yl)isoxazol-5-yl]azetidin-1-yl}-4-(5-methyl-1H-pyrazol-3-ylamino)pyrimidine;
6-methoxy-2-{2-[3-(pyrid-2-yl)isoxazol-5-yl]azetidin-1-yl}-4-(5-methyl-1H-pyrazol-3-ylamino)pyrimidine;
6-methyl-2-{2-[3-(pyrid-2-yl)isoxazol-5-yl]azetidin-1-yl}-4-(5-methyl- 1H-pyrazol-3-ylamino)pyrimidine;
S-6-ethyl-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(2-methylpyrid-3-yl)isoxazol-5-yl]azetidin-1-yl}-4-(5-methyl-1H-pyrazol-3-ylamino)pyrimidine;
S-6-chloro-2-{2-[3-(2-methylpyrid-3-yl)isoxazol-5-yl]azetidin-1-yl}-4-(5-methyl-1H-pyrazol-3-ylamino)pyrimidine;
S-6-morpholino-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(3-ethoxypyrazin-2-yl)isoxazol-5-yl]azetidin-1-yl}-4-(5-methyl-1H-pyrazol-3-ylamino)pyrimidine;
S-6-ethyl-2-{2-[3-(3-ethoxypyrazin-2-yl)isoxazol-5-yl]azetidin-1-yl}-4-(5-methyl-1H-pyrazol-3-ylamino)pyrimidine;
6-ethyl-2-{2-[3-(2-cyanopyrid-3-yl)isoxazol-5-yl]azetidin-1-yl}-4-(5-methyl-1H-pyrazol-3-ylamino)pyrimidine;
S-6-methoxy-2-{2-[3-(2-methylpyrid-3-yl)isoxazol-5-yl]azetidin-1-yl}-4-(5-methyl-1H-pyrazol-3-ylamino)pyrimidine;
S-6-chloro-2-{2-[3-(3-ethylpyrazin-2-yl)isoxazol-5-yl]azetidin-1-yl}4-(5-methyl-1H-pyrazol-3-ylamino)pyrimidine;
S-6-ethoxy-2-{2-[3-(3-ethylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(3-ethylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(2-methylpyrid-3-yl)isoxazol-5-yl]azetidin-1-yl}-4-(5-methyl-1H-pyrazol-3-ylamino)pyrimidine;
S-6-(3-methoxypropoxy)-2-{2-[3-(3-ethylpyrazin-2-yl)isoxazol-5-yl]azetidin-1-yl}-4-(5-methyl-1H-pyrazol-3-ylamino)pyrimidine;
S-6-(3-methoxypropoxy)-2-{2-[3-(2-methylpyrid-3-yl)isoxazol-5-yl]azetidin-1-yl}-4-(5-methyl-1H-pyrazol-3-ylamino)pyrimidine;
S-6-chloro-2-{2-[3-(3-methylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(3-methylpyrazin-2-yl)isoxazol-5-yl]azetidin-1-yl}-4-(5-methyl-1H-pyrazol-3-ylamino)pyrimidine;
S-6-chloro-2-{2-[3-(thiazol-2-yl)isoxazol-5-yl]azetidin-1-yl}-4-(5-methyl-1H-pyrazol-3-ylamino)pyrimidine;
S-6-(2-methoxyethoxy)-2-{2-[3-(thiazol-2-yl)isoxazol-5-yl]azetidin-1-yl}-4-(5-methyl-1H-pyrazol-3-ylamino)pyrimidine;
S-6-(2-methoxyethoxy)-2-{2-[3-(pyrid-2-yl)isoxazol-5-yl]azetidin-1-yl}-4-(5-methyl-1H-pyrazol-3-ylamino)pyrimidine;
S-2-{2-[3-(pyrid-2-yl)isoxazol-5-yl]azetidin-1-yl}-4-(5-tert-butyl-1H-pyrazol-3-ylamino)pyrimidine;
S-6-ethyl-2-{2-[3-(pyrid-2-yl)isoxazol-5-yl]azetidin-1-yl}-4-(5-methyl-1H-pyrazol-3-ylamino)pyrimidine;
S-2-{2-[3-(3-methylpyrazin-2-yl)isoxazol-5-yl]azetidin-1-yl}-4-(5-tert-butyl-1H-pyrazol-3-ylamino)pyrimidine;
S-6-ethyl-2-{2-[3-(3-methylpyrazin-2-yl)isoxazol-5-yl]azetidin-1-yl}-4-(5-methyl-1H-pyrazol-3-ylamino)pyrimidine;
S-6-methyl-2-{2-[3-(3-methylpyrazin-2-yl)isoxazol-5-yl]azetidin-1-yl}-4-(5-tert-butyl-1H-pyrazol-3-ylamino)pyrimidine;
S-6-ethyl-2-{2-[3-(3-methoxypyrazin-2-yl)isoxazol-5-yl]azetidin-1-yl}-4-(5-methyl-1H-pyrazol-3-ylamino)pyrimidine;
S-6-methyl-2-{2-[3-(3-methoxypyrazin-2-yl)isoxazol-5-yl]azetidin-1-yl}-4-(5-tert-butyl-1H-pyrazol-3-ylamino)pyrimidine;
S-6-ethyl-2-{2-[3-(pyrimid-2-yl)isoxazol-5-yl]azetidin-1-yl}-4-(5-methyl-1H-pyrazol-3-ylamino)pyrimidine;
S-6-chloro-2-{2-[3-(pyrimid-2-yl)isoxazol-5-yl]azetidin-1-yl}-4-(5-methyl-1H-pyrazol-3-ylamino)pyrimidine; and
6-ethoxy-2-{2-[3-(pyrimid-2-yl)isoxazol-5-yl]azetidin-1-yl}-4-(5-methyl-1H-pyrazol-3-ylamino)pyrimidine;
and pharmaceutically-acceptable salts thereof.

In yet another aspect, particular compounds of the invention include, for example, any one or more compounds of formula (I) selected from:
6-morpholino-2-{2-[3-(pyrid-2-yl)isoxazol-5-yl]azetidin-1-yl}-4-(5-methyl-1H-pyrazol-3-ylamino)pyrimidine;
6-methoxy-2-{2-[3-(pyrid-2-yl)isoxazol-5-yl]azetidin-1-yl}-4-(5-methyl-1H-pyrazol-3-ylamino)pyrimidine;
6-methyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(2-methylpyrid-3-yl)isoxazol-5-yl]azetidin-1-yl}-4-(5-methyl-1H-pyrazol-3-ylamino)pyrimidine;
S-6-morpholino-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(3-ethoxypyrazin-2-yl)isoxazol-5-yl]azetidin-1-yl}-4-(5-methyl-1H-pyrazol-3-ylamino)pyrimidine;
S-6-ethyl-2-{2-[3-(3-ethoxypyrazin-2-yl)isoxazol-5-yl]azetidin-1-yl}-4-(5-methyl-1H-pyrazol-3-ylamino)pyrimidine;
6-ethyl-2-{2-[3-(2-cyanopyrid-3-yl)isoxazol-5-yl]azetidin-1-yl}-4-(5-1H-pyrazol-3-ylamino)pyrimidine;
S-6-methoxy-2-{2-[3-(2-methylpyrid-3-yl)isoxazol-5-yl]azetidin-1-yl}-4-(5-methyl-1H-pyrazol-3-ylamino)pyrimidine;
S-6-ethoxy-2-{2-[3-(3-ethylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(3-ethylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(2-methylpyrid-3-yl)isoxazol-5-yl]azetidin-1-yl}-4-(5-methyl-1H-pyrazol-3-ylamino)pyrimidine;
S-6-(3-methoxypropoxy)-2-{2-[3-(3-ethylpyrazin-2-yl)isoxazol-5-yl]azetidin-1-yl}-4-(5- methyl-1H-pyrazol-3-ylamino)pyrimidine;
S-6-(3-methoxypropoxy)-2-{2-[3-(2-methylpyrid-3-yl)isoxazol-5-yl]azetidin-1-yl}-4-(5-methyl-1H-pyrazol-3-ylamino)pyrimidine;
S-6-(2-methoxyethoxy)-2-{2-[3-(3-methylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(thiazol-2-yl)isoxazol-5-yl]azetidin-1-yl}-4-(5-methyl-1H-pyrazol-3-ylamino)pyrimidine;
S-6-(2-methoxyethoxy)-2-{2-[3-(pyrid-2-yl)isoxazol-5-yl]azetidin-1-yl}-4-(5-methyl-1H-pyrazol-3-ylamino)pyrimidine;
S-2-{2-[3-(pyrid-2-yl)isoxazol-5-yl]azetidin-1-yl}-4-(5-tert-butyl-1H-pyrazol-3-ylamino)pyrimidine;
S-6-ethyl-2-{2-[3-(pyrid-2-yl)isoxazol-5-yl]azetidin-1-yl}-4-(5-methyl-1H-pyrazol-3-ylamino)pyrimidine;
S-2-{2-[3-(3-methylpyrazin-2-yl)isoxazol-5-yl]azetidin-1-yl}-4-(5-tert-butyl-1H-pyrazol-3-ylamino)pyrimidine;
S-6-ethyl-2-{2-[3-(3-methylpyrazin-2-yl)isoxazol-5-yl]azetidin-1-yl}-4-(5-methyl-1H-pyrazol-3-ylamino)pyrimidine;
S-6-methyl-2-{2-[3-(3-methylpyrazin-2-yl)isoxazol-5-yl]azetidin-1-yl}4-(5-tert-butyl-1H-pyrazol-3-ylamino)pyrimidine;
S-6-ethyl-2-{2-[3-(3-methoxypyrazin-2-yl)isoxazol-5-yl]azetidin-1-yl}-4-(5-methyl-1H-pyrazol-3-ylamino)pyrimidine;
S-6-methyl-2-{2-[3-(3-methoxypyrazin-2-yl)isoxazol-5-yl]azetidin-1-yl}-4-(5-tert-butyl-1H-pyrazol-3-ylamino)pyrimidine;
S-6-ethyl-2-{2-[3-(pyrimid-2-yl)isoxazol-5-yl]azetidin-1-yl}-4-(5-methyl-1H-pyrazol-3-ylamino)pyrimidine; and
6-ethoxy-2-{2-[3-(pyrimid-2-yl)isoxazol-5-yl]azetidin-1-yl}-4-(5-methyl-1H-pyrazol-3-ylamino)pyrimidine;
and pharmaceutically-acceptable salts thereof.

In yet another aspect, particular compounds of the invention include, for example, any one or more compounds of formula (I) selected from:
6-morpholino-2-{2-[3-(pyrid-2-yl)isoxazol-5-yl]azetidin-1-yl}-4-(5-methyl-1H-pyrazol-3-ylamino)pyrimidine;
6-methoxy-2-{2-[3-(pyrid-2-yl)isoxazol-5-yl]azetidin-1-yl}-4-(5-methyl-1H-pyrazol-3-ylamino)pyrimidine;
6-methyl-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(2-methylpyrid-3-yl)isoxazol-5-yl]azetidin-1-yl}-4-(5-methyl-1H-pyrazol-3-ylamino)pyrimidine;
S-6-morpholino-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(3-ethoxypyrazin-2-yl)isoxazol-5-yl]azetidin-1-yl}-4-(5-methyl-1H-pyrazol-3-ylamino)pyrimidine;
S-6-ethyl-2-{2-[3-(3-ethoxypyrazin-2-yl)isoxazol-5-yl]azetidin-1-yl}-4-(5-methyl-1H-pyrazol-3-ylamino)pyrimidine;
6-ethyl-2-{2-[3-(2-cyanopyrid-3-yl)isoxazol-5-yl]azetidin-1-yl}-4-(5-methyl-1H-pyrazol-3-ylamino)pyrimidine;
S-6-methoxy-2-{2-[3-(2-methylpyrid-3-yl)isoxazol-5-yl]azetidin-1-yl}-4-(5-methyl-1H-pyrazol-3-ylamino)pyrimidine;
S-6-ethoxy-2-{2-[3-(3-ethylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(3-ethylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(2-methylpyrid-3-yl)isoxazol-5-yl]azetidin-1-yl}-4-(5-methyl-1H-pyrazol-3-ylamino)pyrimidine;
S-6-(3-methoxypropoxy)-2-{2-[3-(3-ethylpyrazin-2-yl)isoxazol-5-yl]azetidin-1-yl}-4-(5-methyl-1H-pyrazol-3-ylamino)pyrimidine;
S-6-(3-methoxypropoxy)-2-{2-[3-(2-methylpyrid-3-yl)isoxazol-5-yl]azetidin-1-yl}-4(5-methyl-1H-pyrazol-3-ylamino)pyrimidine;
S-6-(2-methoxyethoxy)-2-{2-[3-(3-methylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(thiazol-2-yl)isoxazol-5-yl]azetidin-1-yl}-4-(5-methyl-1H-pyrazol-3-ylamino)pyrimidine;
S-6-(2-methoxyethoxy)-2-{2-[3-(pyrid-2-yl)isoxazol-5-yl]azetidin-1-yl}-4-(5-methyl-1H-pyrazol-3-ylamino)pyrimidine;
S-6-ethyl-2-{2-[3-(pyrid-2-yl)isoxazol-5-yl]azetidin-1-yl}-4-(5-methyl-1H-pyrazol-3-lamino)pyrimidine;
S-6-ethyl-2-{2-[3-(3-methylpyrazin-2-yl)isoxazol-5-yl]azetidin-1-yl}-4-(5-methyl-1H-pyrazol-3-ylamino)pyrimidine;
S-6-ethyl-2-{2-[3-(3-methoxypyrazin-2-yl)isoxazol-5-yl]azetidin-1-yl}-4-(5-methyl-1H-pyrazol-3-ylamino)pyrimidine;
S-6-ethyl-2-{2-[3-(pyrimid-2-yl)isoxazol-5-yl]azetidin-1-yl}-4-(5-methyl-1H-pyrazol-3-ylamino)pyrimidine; and
6-ethoxy-2-{2-[3-(pyrimid-2-yl)isoxazol-5-yl]azetidin-1-yl}-4-(5-methyl-1H-pyrazol-3-ylamino)pyrimidine;
and pharmaceutically-acceptable salts thereof.

A compound of formula (I), or a pharmaceutically-acceptable salt thereof, may be prepared by any process known to be applicable to the preparation of chemically-related compounds. Such processes, when used to prepare a compound of formula (I) are provided as a further feature of the invention and are illustrated by the following representative process variants in which, unless otherwise stated, Q¹, Q², R¹, R² and R³ have any of the meanings defined hereinbefore. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

Process (a) the reaction, conveniently in the presence of a suitable base, of a compound of formula (II):

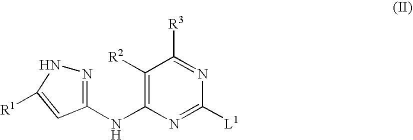

wherein L¹ represents a suitable displaceable group and R¹, R² and R³ are as defined in formula (I) except that any functional group is protected if necessary, with a compound of formula (III):

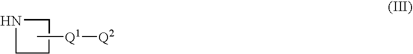

wherein Q¹ and Q² are as defined in formula (I) except that any functional group is protected if necessary; or

Process (b) the reaction, conveniently in the presence of a suitable acid, of a compound of formula (IV):

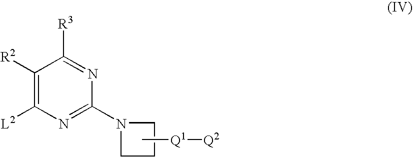

wherein L² is a suitable displaceable group and R², R³, Q¹ and Q² are as defined in formula (I) except that any functional group is protected if necessary, with a pyrazole of formula (V):

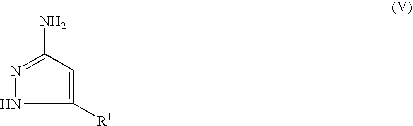

wherein R¹ is as defined in formula (I) except that any functional group is protected if necessary; or

Process (c) the reaction, conveniently in the presence of a suitable base, of a compound of formula (VI):

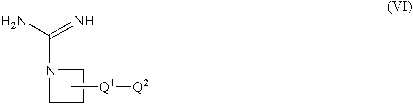

wherein Q¹ and Q² are as defined in formula (I) except that any functional group is protected if necessary, with a compound of formula (VII):

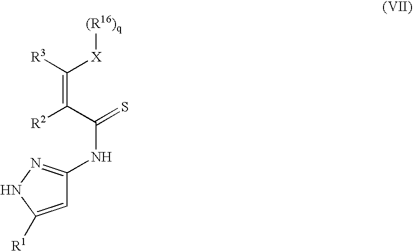

wherein X represents an oxygen atom and q is 1 or X represents a nitrogen atom and q is 2, R¹⁶ is a (C1-C6)alkyl group and R¹, R² and R³ are as defined in formula (I) except that any functional group is protected if necessary; or

Process (d) the reaction of a compound of formula (VIII):

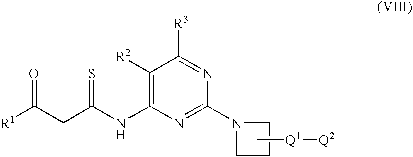

wherein R¹, R², R³, Q¹ and Q² are as defined in formula (I) except that any functional group is protected if necessary, with hydrazine; or

Process (e) for compounds of formula (I) wherein R³ is a (C1-C6)alkoxy, amino, (C1-C6)alkylamino, di-[(C1-C6)alkyl]amino, —OR^{3b}, —SR^{3b}, —NHR^{3b}, N[(C1-C6)alkyl]R^{3b} or —S(O)ₘR^{3a} group wherein m is 0 and R^{3a} and R^{3b} are as defined above (and the group R³ is optionally substituted by at least one group as defined above), the reaction, conveniently in the presence of a suitable base, of a compound of formula (IX):

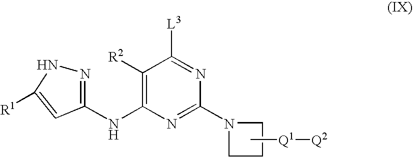

wherein L³ is a suitable displaceable group and R¹, R², Q¹ and Q² are as defined in formula (I) except that any functional group is protected if necessary, with a compound of formula:

H—Xa

wherein Xa represents OR¹⁷, NH₂, NHR¹⁷, N(R¹⁷)₂, OR^{3b}, SR^{3b}, NHR^{3b}, N[(C1-C6)alkyl]R^{3b} and SR^{3a}, wherein R¹⁷ is an, optionally substituted, (C1-C6)alkyl group and R^{3a} and R^{3b} are each as defined above except that any functional group is protected if necessary; or

Process (f) for compounds of formula (I) wherein R³ is (i) an, optionally substituted, saturated monocyclic 5- or 6-membered heterocyclic ring comprising at least one ring nitrogen and, optionally, one or more additional heteroatoms selected from nitrogen, oxygen and sulfur, or (ii) an optionally substituted 2,7-diazaspiro[3.5]nonane group, the reaction, conveniently in the presence of a suitable base, of a compound of formula (IX) as defined above, with (i) a compound of formula (Xb):

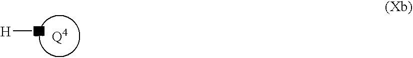

wherein Q⁴ is a saturated monocyclic 5- or 6-membered heterocyclic ring optionally comprising one or more heteroatoms selected from nitrogen, oxygen and sulfur in addition to the nitrogen atom shown above, which ring is optionally substituted by at least one group as defined above, or

with (ii) an optionally substituted 2,7-diazaspiro[3.5]nonane; or

Process (g) for compounds of formula (I) wherein R³ is a (C2-C6)alkenyl or (C2-C6)alkynyl group, and the group R³is optionally substituted by at least one group as defined above, the reaction, conveniently in the presence of a suitable base and a suitable catalyst, of a compound of formula (IX) as defined above, with a compound of formula (Xc) or of formula (Xc′):

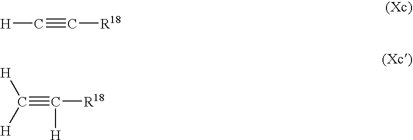

wherein R¹⁸ is selected from hydrogen and an, optionally substituted, (1-4C)alkyl or (C1-C4)alkoxycarbonyl group; or

Process (h) for compounds of formula (I) wherein R³ is attached to the pyrimidine ring through a carbon atom, the reaction, conveniently in the presence of a suitable catalyst, of a compound of formula (IX) as defined above, with a compound of the formula:

M-R³

wherein R³² is appropriately selected from the R³ groups as defined above and M is a metallic group, such as ZnBr, B(OH)₂, CuCN or SnBu₃; or

Process (i) for compounds of formula (I) wherein R³ is a (C1-C6)alkoxycarbonyl group (and the group R³ is optionally substituted by at least one group as defined above), the reaction, conveniently in the presence of a suitable acid, of a compound of formula (X):

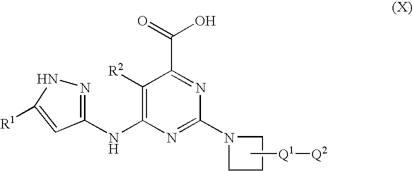

wherein R¹, R², Q¹ and Q² are as defined in formula (I) except that any functional group is protected if necessary, with a compound of formula:

H—O—(C1-C6)alkyl

wherein the (C1-C6)alkyl group is optionally substituted by at least one group as defined above as a substituent for R³ and any functional group is protected if necessary; or

Process (j) for compounds of formula (I) wherein R³ is a 5-membered heteroaromatic ring comprising at least one heteroatom selected from nitrogen, oxygen and sulfur (and the group R³ is optionally substituted by at least one group as defined above), an internal condensation reaction using an appropriate starting material and a suitable dehydrating agent.

For example, for compounds of formula (I) wherein R³ is a 1,3,4-oxadiazole group, the reaction of a compound of formula (XI):

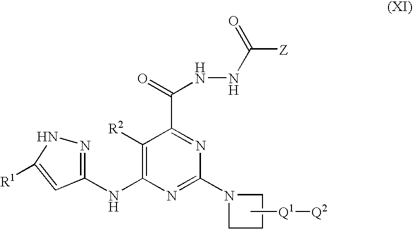

wherein Z represents any suitable substituent for R³ as defined above and R¹, R², Q¹ and Q² are as defined in formula (I) except that any functional group is protected if necessary, with a suitable dehydrating agent, such as (methoxycarbonylsulfamoyl)triethylammonium hydroxide; or

Process (k) for compounds of formula (I) wherein R³ is a (C1-C6)alkyl, (C3-C6)alkenyl, (C3-C6)alkynyl or (C1-C6)alkoxy group substituted by at least one group as defined above, reacting a compound of formula (XII):

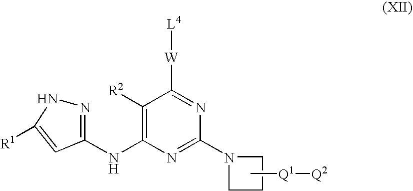

wherein L⁴ is a suitable displaceable group, W is an optionally substituted (C1-C6)alkyl, (C3-C6)alkenyl, (C3-C6)alkynyl or (C1-C6)alkoxy group and R¹, R², Q¹ and Q² as defined in formula (I) except that any functional group is protected if necessary, with a compound of formula H—Xa, (Xb), (Xc), (Xc′) or M-R³ as defined above;

and optionally after process (a), (b), (c), (d) (e), (f), (g), (h), (i), (j) or (k) carrying out one or more of the following:
converting the compound obtained to a further compound of the invention
forming a pharmaceutically-acceptable salt of the compound.

### Process (a)

### Reaction Conditions for Process (a)

A suitable displaceable group L¹ in the compound of formula (II) is for example a halogeno or a sulfonyloxy group, for example a fluoro, chloro, methylsulfonyloxy or toluene-4-sulfonyloxy group. A particular group L¹ is fluoro, chloro or methylsulfonyloxy.

Process (a) conveniently may be carried out in the presence of a suitable base and/or in the presence of a suitable Lewis acid. A suitable base is, for example, an organic amine base such as pyridine, 2,6-lutidine, collidine, 4-dimethylaminopyridine, triethylamine, di-isopropylethylamine, N-methylmorpholine or diazabicyclo[5.4.0]undec-7-ene, or, for example, an alkali or alkaline earth metal carbonate, such as sodium carbonate, potassium carbonate, cesium carbonate or calcium carbonate, or, for example, an alkali metal hydride, such as sodium hydride. A particular base is an organic amine base, for example di-isopropylethylamine. A suitable Lewis acid is zinc acetate.

Process (a) may conveniently be carried out in the presence of a suitable inert solvent or diluent for example a ketone such as acetone or an alcohol such as ethanol, butanol, isopropanol or n-hexanol or an aromatic hydrocarbon such as toluene or N-methyl pyrrolid-2-one and at a temperature in the range from 0° C. to reflux, particularly reflux.

Process (a) may alternatively conveniently be carried out under standard Buchwald conditions (see, for example, J. Am. Chem. Soc., 118, 7215; J. Am. Chem. Soc., 119, 8451; J. Org. Chem., 62, 1568 and 6066). For example, process (a) may conveniently be carried out in the presence of palladium acetate, in a suitable inert solvent or diluent for example an aromatic solvent such as toluene, benzene or xylene, in the presence of a suitable base, for example an inorganic base such as caesium carbonate or an organic base such as potassium-t-butoxide and in the presence of a suitable ligand such as 2,2′-bis(diphenylphosphino)-1,1′-binaphthyl and at a temperature in the range from 25 to 80° C.

### Starting Materials for Process (a)

A compound of formula (II) may be obtained by conventional procedures. For example, a compound of formula (II) may be obtained by the reaction, conveniently in the presence of a suitable base, of a pyrimidine of formula (IIa):

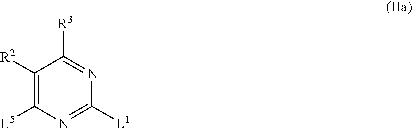

wherein L⁵ is a suitable displaceable group and L¹, R² and R³ have any of the meanings defined hereinbefore except that any functional group is protected if necessary, with a pyrazole of formula (V):

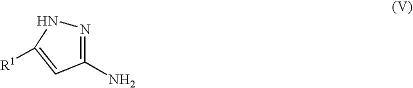

wherein R¹ has any of the meanings defined hereinbefore except that any functional group is protected if necessary.

A suitable displaceable group L⁵ in the compound of formula (IIa) is, for example, a halogeno or a sulfonyloxy group, for example a fluoro, chloro, methylsulfonyloxy or toluene-4-sulfonyloxy group. A particular group L⁵ is chloro.

A suitable base for the reaction of a pyrimidine of formula (IIa) and a pyrazole of formula (V) includes, for example, an alkali or alkaline earth metal carbonate, such as sodium carbonate, potassium carbonate, cesium carbonate or calcium carbonate or an organic amine base such as di-isopropylethylamine.

The reaction may conveniently be carried out in the presence of a suitable inert solvent or diluent for example a ketone such as acetone or an alcohol such as ethanol, butanol or n-hexanol or an aromatic hydrocarbon such as toluene or N-methyl pyrrolid-2-one. The reaction is conveniently carried out at a temperature in the range of, for example, 10 to 150° C., particularly at room temperature.

Pyrimidines of formula (IIa) and pyrazoles of formula (V) are commercially available compounds or they are known in the literature, or they can be prepared by standard processes known in the art.

A compound of formula (III) may be obtained by conventional procedures. For example, when Q¹ is isoxazole, a compound of formula (III) may be obtained as illustrated in Reaction Scheme 1:

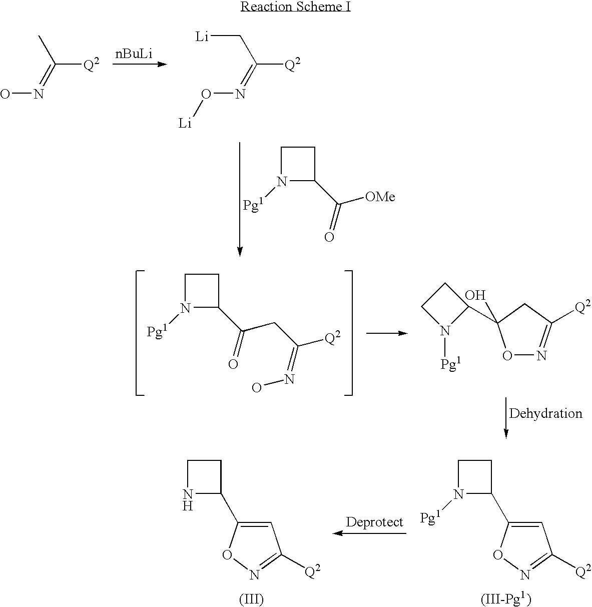

In Reaction Scheme 1, Pg¹ is a suitable protecting group, such as, for example, tert-butoxycarbonyl. The group Q² is as previously defined. For example, Q² may be pyridyl such as pyrid-2-yl).

Alternatively, for example, when Q¹ is isoxazole, a compound of formula (III) may be obtained as illustrated in Reaction Scheme 2:

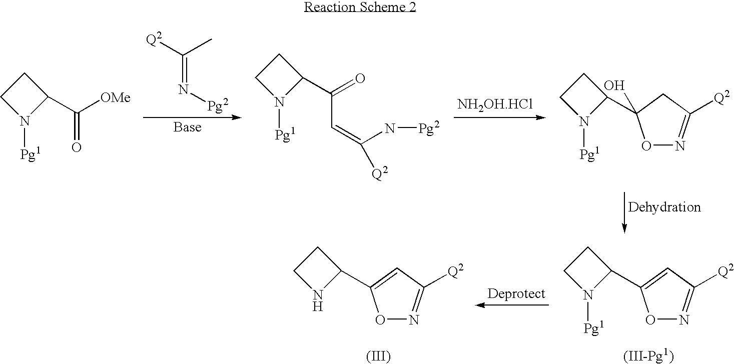

In Reaction Scheme 2, Pg¹ is a suitable protecting group as described above. Similarly, Pg² is a suitable protecting group such as, for example, cyclohexyl. The group Q² is as previously defined.

Alternatively, for example, when Q¹ is isoxazole, a compound of formula (III) may be obtained as illustrated in Reaction Scheme 3:

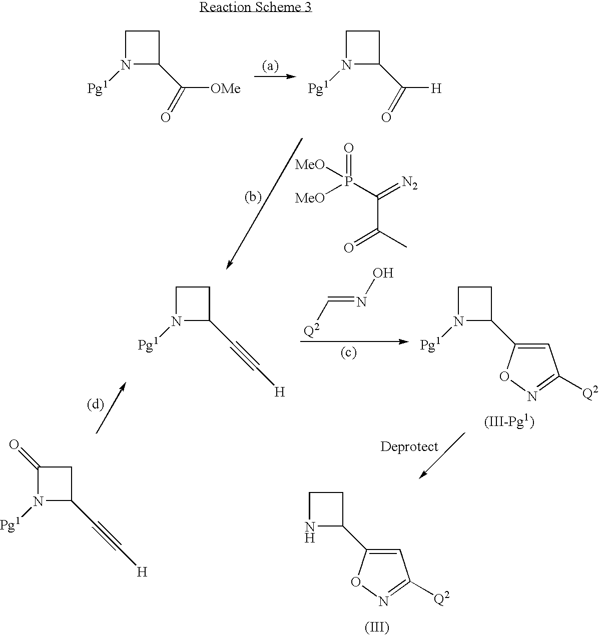

In Reaction Scheme 3, Pg¹ is a suitable protecting group as described above. The group Q² is as previously defined.

In Reaction Scheme 3, step (a) may conveniently be effected by a suitable reducing agent, such as diisobutylaluminium hydride. Step (a) may conveniently be carried out in the presence of a suitable inert solvent or diluent, for example an ether or an aromatic hydrocarbon such as toluene or a chlorinated hydrocarbon such as dichloromethane and at a temperature in the range of, for example, from −78° C. to 25° C.

Step (b) may conveniently be carried out in the presence of a suitable inert solvent or diluent for example a chlorinated hydrocarbon such as dichloromethane and at a temperature in the range of, for example, from −20° C. to 50° C.

Step (c) may conveniently be effected by treatment with a suitable chlorinating agent, such as N-chlorosuccinimide to give an α-chloroaldyde oxime intermediate, and then a suitable base, such as triethylamine, to give a nitrile oxide intermediate which takes part in a 3+2 cycloaddition reaction. Alternatively, the oxime (Q²-CH═N—OH) may be directly transformed into a nitrile oxide intermediate by treatment with sodium hypochlorite. Such reactions may conveniently be carried out in the presence of a suitable inert solvent or diluent, for example a chlorinated hydrocarbon such as dichloromethane and at a temperature in the range of, for example, from −20° C. to 50° C.

Step (d) may conveniently be effected by a suitable reducing agent, such as borane, diisobutylaluminium hydride or lithium aluminium hydride. Step (d) may conveniently be carried out in the presence of a suitable inert solvent or diluent, for example an ether or aromatic hydrocarbon such as toluene or a chlorinated hydrocarbon such as dichloromethane and at a temperature in the range of, for example, from −50° C. to 100° C.

In each of Reaction Schemes 1, 2 and 3, the protecting group may be removed by any convenient method as described in the literature or known to the skilled chemist as appropriate for the removal of the particular protecting group used.

### Process (b)

### Reaction Conditions for Process (b)

A suitable displaceable group L² in a compound of formula (IV) is, for example, halogeno or a sulfonyloxy group, for example fluoro, chloro, methanesulfonyloxy or toluene-4-sulfonyloxy.

Process (b) is conveniently carried out in the presence of a suitable acid. A suitable acid is, for example, an inorganic acid such as anhydrous hydrogen chloride.

Process (b) may conveniently be carried out in the presence of a suitable inert solvent or diluent for example a ketone such as acetone or an alcohol such as ethanol, butanol or n-hexanol or an aromatic hydrocarbon such as toluene or N-methyl pyrrolid-2-one and at a temperature in the range from 0° C. to reflux, particularly reflux.

Process (b) may alternatively conveniently be carried out under standard Buchwald conditions as discussed above for process (a).

### Starting Materials for Process (b)

A compound of formula (IV) may be prepared using conventional methods, for example as discussed above.

Pyrazoles of formula (V) are commercially available compounds or they are known in the literature, or they can be prepared by standard processes known in the art.

### Process (c)

### Reaction Conditions for Process (c)

Process (c) is conveniently carried out in a suitable inert solvent or diluent such as N-methylpyrrolidinone or butanol at a temperature in the range from 100 to 200° C., in particular in the range from 150 to 170° C. The reaction is preferably conducted in the presence of a suitable base such as, for example, sodium methoxide or potassium carbonate.

### Starting Materials for Process (c)

Compounds of the formulae (VI) and (VII) are commercially available compounds or they are known in the literature, or they can be prepared by standard processes known in the art.

### Process (d)

### Reaction Conditions for Process (d)

Process (d) is conveniently carried out in a suitable inert solvent or diluent, for example, an alcohol such as ethanol or butanol at a temperature in the range from 50 to 120° C., in particular in the range from 70 to 100° C.

### Starting Materials for Process (d)

A compound of formula (VIII) may be prepared using conventional methods, for example as discussed above.

Hydrazine is a commercially available compound.

### Process (e)

### Reaction Conditions for Process (e)

A suitable displaceable group L³ in a compound of formula (IX) is, for example, halogeno or a sulfonyloxy group, for example fluoro, chloro, methanesulfonyloxy or toluene-4-sulfonyloxy.

Process (e) is conveniently carried out in the presence of a suitable base. A suitable base is, for example, sodium hydride or an organic amine base such as diisopropylethylamine. Another suitable base is an alkali metal alkoxide, for example sodium methoxide or sodium ethoxide.

Process (e) is conveniently carried out in the presence of a suitable inert solvent or diluent, for example a ketone such as acetone, or an alcohol such as methanol, ethanol, butanol or n-hexanol, or an aromatic hydrocarbon such as toluene or N-methyl pyrrolid-2-one.

Process (e) is conveniently carried out at a temperature in the range from 0° C. to reflux, particularly reflux. Conveniently, process (e) may also be performed by heating the reactants in a sealed vessel using a suitable heating apparatus such as a microwave heater.

### Starting Materials for Process (e)

A compound of formula (IX) may be prepared using conventional methods, for example as discussed above.

Compounds of the formula H—Xa are commercially available compounds or they are known in the literature, or they can be prepared by standard processes known in the art.

### Process (f)

### Reaction Conditions for Process (f)

The reaction of process (f) is conveniently carried out using analogous conditions to those described above for process (e).

### Starting Materials for Process (f)

A compound of formula (IX) may be prepared using conventional methods, for example as discussed above.

Compounds of the formula Xb are commercially available compounds or they are known in the literature, or they can be prepared by standard processes known in the art. 2,7-diazaspiro[3.5]nonane (and substituted derivatives thereof) is a commercially available compound.

### Process (g)

### Reaction Conditions for Process (g)

Process (g) is conveniently carried out in the presence of a suitable base. A suitable base is, for example, an organic amine base, such as for example triethylamine or diisopropylethylamine.

Process (g) is conveniently carried out in the presence of a suitable catalyst. A suitable catalyst is, for example, copper iodide/palladium (II) chloride-bis(triphenyl)phosphine.

Process (g) is conveniently carried out in the presence of a suitable inert solvent or diluent for example acetonitrile, THF or dioxane and at a temperature in the range from 0° C. to reflux, particularly reflux. Conveniently, Process (g) may also be performed by heating the reactants in a sealed vessel using a suitable heating apparatus such as a microwave heater.

### Starting Materials for Process (g)

A compound of formula (IX) may be prepared using conventional methods, for example as discussed above.

Compounds of the formula Xc and Xc′ are commercially available compounds or they are known in the literature, or they can be prepared by standard processes known in the art.

### Process (h)

### Reaction Conditions for Process (h)

Process (h) is conveniently carried out in the presence of a suitable catalyst. A suitable catalyst is, for example, a palladium (0) catalyst, such as for example tetrakis(triphenyl)phosphine palladium(0). As a person skilled in the art would appreciate, the palladium (0) catalyst may be prepared in situ.

Process (h) is conveniently carried out in the presence of a suitable inert solvent or diluent for example THF or dioxane and at a temperature in the range from 0° C. to reflux, particularly reflux.

### Starting Materials for Process (h)

A compound of formula (IX) may be prepared using conventional methods, for example as discussed above.

Compounds of the formula M-R³ are commercially available compounds or they are known in the literature, or they can be prepared by standard processes known in the art.

### Process (i)

### Reaction Conditions for Process (i)

Process (i) is conveniently carried out in the presence of a suitable acid. A suitable acid is, for example, concentrated sulfuric acid.

Process (i) is conveniently carried out in the absence of an inert solvent or diluent and at a temperature in the range from room temperature to reflux, particularly reflux.

### Starting Materials for Process (i)

A compound of formula (X) may be prepared using conventional methods, for example as discussed above.

Compounds of the formula H—O—(C1-C6)alkyl are commercially available compounds or they are known in the literature, or they can be prepared by standard processes known in the art.

### Process (j)

### Reaction Conditions for Process (j)

Process (j) is conveniently carried out in the presence of a suitable inert solvent or diluent, such as for example dichloromethane, THF or dioxane. Process (j) is conveniently carried out at a temperature in the range from 0° C. to reflux, particularly reflux.

### Starting Materials for Process (j)

A compound of formula (XI) may be prepared using conventional methods, for example as discussed above.

Suitable dehydrating agents are commercially available compounds or they are known in the literature, or they can be prepared by standard processes known in the art.

### Process (k)

### Reaction Conditions for Process (k)

A suitable displaceable group L⁴ in a compound of formula (XII) is, for example, halogeno or a sulfonyloxy group, for example fluoro, chloro, methanesulfonyloxy or toluene-4-sulfonyloxy.

The reaction of process (k) is conveniently carried out using analogous conditions to those described above for process (e).

### Starting Materials for Process (k)

A compound of formula (XII) may be prepared using conventional methods, for example as discussed above.

Compounds of the formula H—Xa, (Xb), (Xc), (Xc′) or M-R³ are commercially available compounds or they are known in the literature, or they can be prepared by standard processes known in the art.

As stated above, compounds of formulae (II), (III), (IV), (V), (VI), (VII), (VIII), HXa, (Xb), (Xc), (Xc′) and M-R³ are either commercially available, are known in the literature or may be prepared using known techniques. For example, these compounds may be prepared by analogous processes to those described in WO 03/048133. Examples of preparation methods for certain of these compounds are given hereinafter in the examples.

It will be appreciated that compounds of formula (I) can be converted into further compounds of formula (I) using standard procedures conventional in the art, for example by means of conventional substitution reactions or of conventional functional group modifications either prior to or immediately following the processes mentioned above, and such procedures are included in the process aspect of the invention.

Examples of the types of conversion reactions that may be used include introduction of a substituent by means of an aromatic substitution reaction or of a nucleophilic substitution reaction, reduction of substituents, alkylation of substituents and oxidation of substituents. The reagents and reaction conditions for such procedures are well known in the chemical art.

Particular examples of aromatic substitution reactions include the introduction of a nitro group using concentrated nitric acid; the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of nucleophilic substitution reactions include the introduction of an alkoxy group or of an alkylamino group, a dialkyamino group or a N-containing heterocycle using standard conditions. Particular examples of reduction reactions include the reduction of a carbonyl group to a hydroxy group with sodium borohydride or of a nitro group to an amino group by catalytic hydrogenation with a nickel catalyst or by treatment with iron in the presence of hydrochloric acid with heating; and particular examples of oxidation reactions include oxidation of alkylthio to alkylsulfinyl or alkylsulfonyl. Other conversion reactions that may be used include the acid catalysed esterification of carboxylic acids with alcohols.

An example of a suitable conversion reaction is the conversion of a compound of formula (I) wherein R³ is a (C1-C6)alkenyl group to a compound of formula (I) wherein R³ is a (C1-C6)alkyl group substituted by a di-[(C1-C6)alkyl]amino group or by a saturated monocyclic 4- to 7-membered ring, which ring comprises nitrogen and one or more heteroatoms independently selected from nitrogen, oxygen and sulfur. Such a conversion may be achieved using standard procedures, for example by conversion of the alkenyl group to a dihydroxyalkyl group with osmium tetroxide, oxidation to the corresponding ketone with a suitable oxidising agent (for example sodium periodate) and conversion of the ketone group to the desired substituent as defined above by reaction with the appropriate amine in the presence of a suitable reducing agent (for example sodium cyanoborohydride).

Another example of a suitable conversion reaction is the conversion of a compound of formula (I) wherein R³ is an optionally substituted (C1- C6)alkoxycarbonyl group to a compound of formula (I) wherein R³is an optionally substituted carbamoyl, (C1-C6)alkylcarbamoyl or di-[(C1-C6)alkyl]carbamoyl group or an optionally substituted —C(O)R^{3b} group, wherein R^{3b} is as defined above. Such a conversion may be achieved using standard procedures, for example by reaction of the compound of formula (I) wherein R³ is an optionally substituted (C1-C6)alkoxycarbonyl group with ammonia, with an optionally substituted primary, secondary or tertiary amine or with an optionally substituted H—R^{3b} group. As the skilled person would appreciate, this conversion could be conducted starting from the carboxylic acid and preparing an activated ester, for example using 4-(4,6-dimethoxy[1,3,5]triazin-2-yl)-4-methyl-molpholinium chloride, which may then be reacted with the necessary amine.

Another example of a suitable conversion reaction is the conversion of a compound of formula (I) wherein R³ is a (C1-C6)alkoxycarbonyl group to a compound of formula (I) wherein R³ is a hydroxy-(C1-C6)alkyl group. Such a conversion may be achieved using standard procedures, for example by reduction using lithium borohydride or lithium aluminium hydride.

It will be appreciated that the preparation of compounds of formula (I) may involve, at various stages, the addition and removal of one or more protecting groups. The protecting groups used in the processes above may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1 to 4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

A carboxy protecting group may be the residue of an ester-forming aliphatic or arylaliphatic alcohol or of an ester-forming silanol (the said alcohol or silanol preferably containing 1 to 20 carbon atoms). Examples of carboxy protecting groups include straight or branched chain (1 to 12C)alkyl groups (for example isopropyl, and tert-butyl); lower alkoxy- lower alkyl groups (for example methoxymethyl, ethoxymethyl and isobutoxymethyl); lower acyloxy-lower alkyl groups, (for example acetoxymethyl, propionyloxymethyl, butyryloxymethyl and pivaloyloxymethyl); lower alkoxycarbonyloxy-lower alkyl groups (for example 1-methoxycarbonyloxyethyl and 1-ethoxycarbonyloxyethyl); aryl-lower alkyl groups (for example benzyl, 4-methoxybenzyl, 2-nitrobenzyl, 4-nitrobenzyl, benzhydryl and phthalidyl); tri(lower alkyl)silyl groups (for example trimethylsilyl and tert-butyldimethylsilyl); tri(lower alkyl)silyl-lower alkyl groups (for example trimethylsilylethyl); and (2-6C)alkenyl groups (for example allyl). Methods particularly appropriate for the removal of carboxy protecting groups include for example acid-, base-, metal- or enzymically-catalysed cleavage.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); lower alkanoyloxyalkyl groups (for example pivaloyloxymethyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl, hydrogenation for groups such as benzyl and photolytically for groups such as 2-nitrobenzyloxycarbonyl. For example a tert butoxycarbonyl protecting group may be removed from an amino group by an acid catalysed hydrolysis using trifluoroacetic acid.

The reader is referred to Advanced Organic Chemistry, 4th Edition, by J. March, published by John Wiley & Sons 1992, for general guidance on reaction conditions and reagents and to Protective Groups in Organic Synthesis, 2^{nd} Edition, by T. Green et al., also published by John Wiley & Son, for general guidance on protecting groups.

When a pharmaceutically-acceptable salt of a compound of formula (I) is required, for example an acid-addition salt, it may be obtained by, for example, reaction of said compound with a suitable acid using a conventional procedure. When it is desired to obtain the free base from a salt of the compound of formula (I), a solution of the salt may be treated with a suitable base, for example, an alkali or alkaline earth metal carbonate or hydroxide, for example sodium carbonate, potassium carbonate, calcium carbonate, sodium hydroxide or potassium hydroxide.

As mentioned hereinbefore some of the compounds according to the present invention may contain one or more chiral centers and may therefore exist as stereoisomers. Stereoisomers may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, or example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free for other stereoisomers, for example containing less than 20%, particularly less than 10% and more particularly less than 5% by weight of other stereoisomers.

In the section above relating to the preparation of the compounds of formula (I), the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Certain intermediates used in the processes described above are novel and form a further feature of the present invention. Accordingly there is provided a compound selected from a compound the formulae (III) and (III-Pg¹) as hereinbefore defined, or a salt thereof. The intermediate may be in the form of a salt of the intermediate. Such salts need not be a pharmaceutically-acceptable salt. For example it may be useful to prepare an intermediate in the form of a pharmaceutically non-acceptable salt if, for example, such salts are useful in the manufacture of a compound of formula (I).

In one aspect, particular intermediate compounds of the invention include, for example, one or more intermediate compounds of the formula (III) selected from:
2-[3-(2-pyridyl)isoxazol-5-yl]azetidine;
2-[3-(2-methylpyrid-3-yl)isoxazol-5-yl]azetidine;
2-[3-(3-ethoxypyrazin-2-yl)isoxazol-5-yl]azetidine;
2-[3-(2-cyanopyrid-3-yl)isoxazol-5-yl]azetidine;
2-[3-(3-ethylpyrazin-2-yl)isoxazol-5-yl]azetidine;
2-[3-(3-methylpyrazin-2-yl)isoxazol-5-yl]azetidine;
2-[3-(thiazol-2-yl)isoxazol-5-yl]azetidine;
2-[3-(3-methoxypyrazin-2-yl)isoxazol-5-yl]azetidine; and
2-[3-(pyrimid-2-yl)isoxazol-5-yl]azetidine;
and salts thereof.

In another aspect, particular intermediate compounds of the invention include, for example, one or more intermediate compounds of the formula (III) selected from:
S-2-[3-(pyrid-2-yl)isoxazol-5-yl]azetidine;
S-2-[3-(2-methylpyrid-3-yl)isoxazol-5-yl]azetidine;
S-2-[3-(3-ethoxypyrazin-2-yl)isoxazol-5-yl]azetidine;
S-2-[3-(2-cyanopyrid-3-yl)isoxazol-5-yl]azetidine;
S-2-[3-(3-ethylpyrazin-2-yl)isoxazol-5-yl]azetidine;
20 S-2-[3-(3-methylpyrazin-2-yl)isoxazol-5-yl]azetidine;
S-2-[3-(thiazol-2-yl)isoxazol-5-yl]azetidine;
S-2-[3-(3-methoxypyrazin-2-yl)isoxazol-5-yl]azetidine; and
S-2-[3-(pyrimid-2-yl)isoxazol-5-yl]azetidine;
and salts thereof.

In another aspect, particular intermediate compounds of the invention include, for example, one or more intermediate compounds of the formula (III-Pg¹) selected from:
N-(tert-butoxycarbonyl)-2-[3-(2-pyridyl)isoxazol-5-yl]azetidine;
N-(tert-butoxycarbonyl)-2-[3-(2-methylpyrid-3-yl)isoxazol-5-yl]azetidine;
N-(tert-butoxycarbonyl)-2-[3-(3-ethoxypyrazin-3-yl)isoxazol-5-yl]azetidine;
N-(tert-butoxycarbonyl)-2-[3-(2-chloropyrid-3-yl)isoxazol-5-yl]azetidine;
N-(tert-butoxycarbonyl)-2-[3-(2-cyanopyrid-3-yl)isoxazol-5-yl]azetidine;
N-(tert-butoxycarbonyl)-2-[3-(3-ethylpyrazin-2-yl)isoxazol-5-yl]azetidine;
N-(tert-butoxycarbonyl)-2-[3-(3-methylpyrazin-2-yl)isoxazol-5-yl]azetidine;
N-(tert-butyloxycarbonyl)-2-[3-(thiazol-2-yl)isoxazol-5-yl]azetidine;
N-(tert-butoxycarbonyl)-2-[3-(3-methoxypyrazin-2-yl)isoxazol-5-yl]azetidine; and
N-(tert-butoxycarbonyl)-2-[3-(pyrimid-2-yl)isoxazol-5-yl]azetidine;
and salts thereof.

In yet another aspect, particular intermediate compounds of the invention include, for example, one or more intermediate compounds of the formula (III-Pg¹) selected from:
S—N-(tert-butoxycarbonyl)-2-[3-(2-pyridyl)isoxazol-5-yl]azetidine;
S—N-(tert-butoxycarbonyl)-2-[3-(2-methylpyrid-3-yl)isoxazol-5-yl]azetidine;
S—N-(tert-butoxycarbonyl)-2-[3-(3-ethoxypyrazin-3-yl)isoxazol-5-yl]azetidine;
S—N-(tert-butoxycarbonyl)-2-[3-(2-chloropyrid-3-yl)isoxazol-5-yl]azetidine;
S—N-(tert-butoxycarbonyl)-2-[3-(2-cyanopyrid-3-yl)isoxazol-5-yl]azetidine;
S—N-(tert-butoxycarbonyl)-2-[3-(3-ethylpyrazin-2-yl)isoxazol-5-yl]azetidine;
S—N-(tert-butoxycarbonyl)-2-[3-(3-methylpyrazin-2-yl)isoxazol-5-yl]azetidine;
S—N-(tert-butyloxycarbonyl)-2-[3-(thiazol-2-yl)isoxazol-5-yl]azetidine;
S—N-(tert-butoxycarbonyl)-2-[3-(3-methoxypyrazin-2-yl)isoxazol-5-yl]azetidine; and
S—N-(tert-butoxycarbonyl)-2-[3-(pyrimid-2-yl)isoxazol-5-yl]azetidine;
and salts thereof.

The activity and selectivity of compounds according to the invention may be determined using an appropriate assay as described, for example, in WO 03/048133, and detailed below.

### Biological Assays

### IGF-1R Kinase Assay

a) Protein cloning, expression and purification

A DNA molecule encoding a fusion protein containing glutathione-S-transferase (GST), thrombin cleavage site and IGF-1R intracellular domain (amino-acids 930-1367) and subsequently referred to as GST-IGFR, was constructed and cloned into pFastBac1 (Life Technologies Ltd, UK) using standard molecular biology techniques (Molecular Cloning—A Laboratory Manual, Second Edition 1989; Sambrook, Fritsch and Maniatis; Cold Spring Harbour Laboratory Press).

Production of recombinant virus was performed following the manufacturer's protocol.

Briefly, the pFastBac-1 vector containing GST-IGFR was transformed into E. coli DH10Bac cells containing the baculovirus genome (bacmid DNA) and via a transposition event in the cells, a region of the pFastBac vector containing gentamycin resistance gene and the GST-IGFR expression cassette including the baculovirus polyhedrin promoter was transposed directly into the bacmid DNA. By selection on gentamycin, kanamycin, tetracycline and X-gal, resultant white colonies should contain recombinant bacmid DNA encoding GST-IGFR. Bacmid DNA was extracted from a small scale culture of several BH10Bac white colonies and transfected into Spodoptera frugiperda Sf21 cells grown in TC100 medium (Life Technologies Ltd, UK) containing 10% serum using CellFECTIN reagent (Life Technologies Ltd, UK) following the manufacturer's instructions. Virus particles were harvested by collecting cell culture medium 72 hrs post transfection. 0.5 ml of medium was used to infect 100 ml suspension culture of Sf21s containing 1×10⁷ cells/ml. Cell culture medium was harvested 48 hrs post infection and virus titre determined using a standard plaque assay procedure. Virus stocks were used to infect Sf9 and “High 5” cells at a multiplicity of infection (MOI) of 3 to ascertain expression of recombinant GST-IGFR.

The GST-IGFR protein was purified by affinity chromatography on Glutathione-Sepharose followed by elution with glutathione. Briefly, cells were lysed in 50 mM HEPES pH 7.5 (Sigma, H3375), 200 mM NaCl (Sigma, S7653), Complete Protease Inhibitor cocktail (Roche, 1 873 580) and 1 mM DTT (Sigma, D9779), hereinafter referred to as lysis buffer. Clarified lysate supernatant was loaded through a chromatography column packed with Glutathione Sepharose (Amersham Pharmacia Biotech UK Ltd.). Contaminants were washed from the matrix with lysis buffer until the UV absorbance at 280 nm returned to the baseline. Elution was carried out with lysis buffer containing 20 mM reduced glutathione (Sigma, D2804) and fractions containing the GST fusion protein were pooled and dialysed into a glycerol-containing buffer comprising 50 mM HEPES, pH 7.5, 200 mM NaCl, 10% glycerol (v/v), 3 mM reduced glutathione and 1 mM DTT.

### b) Kinase Activity Assay

The activity of the purified enzyme was measured by phosphorylation of a synthetic poly GluAlaTyr (EAY) 6:3:1 peptide (Sigma-Aldrich Company Ltd, UK, P3899) using an ELISA detection system in a 96-well format.

### b.i) Reagents Used

### Stock Solutions

| 200 | mM | HEPES, pH 7.4 | stored at 4° C. | (Sigma, H3375) |
|---|---|---|---|---|
| 1M | | DTT | stored at −20° C. | (Sigma, D9779) |
| 100 | mM | Na₃VO₄ | stored at 4° C. | (Sigma, S6508) |
| 1M | | MnCl₂ | stored at 4° C. | (Sigma, M3634) |
| 1 | mM | ATP | stored at −20° C. | (Sigma, A3377) |
| Neat | | Triton X-100 | stored at room | (Sigma, T9284) |
| | | | temperature | |
| 10 | mg/ml | BSA | stored at 4° C. | (Sigma, A7888) |

### Enzyme Solution

GST-IGF-1R fusion protein at 75 ng/ml in 100 mM HEPES, pH 7.4, 5mM DTT, 0.25 mM Na₃VO₄, 0.25% Triton X-100, 0.25 mg/ml BSA, freshly prepared.

### Co-Factor Solution

100 mM HEPES, pH 7.4, 60 mM MnCl₂, 5 mM ATP.

### Poly EAY Substrate

Sigma substrate poly (Glu, Ala, Tyr) 6:3:1 (P3899). Made up to 1 mg/ml in PBS and stored at −20° C.

### Assay Plates

Nunc Maxisorp 96 well immunoplates (Life Technologies Ltd, UK).

### Antibodies

Anti-phosphotyrosine antibody, monoclonal from Upstate Biotechnology Inc., NY, USA (UBI 05-321). Dilute 3 μl in 11 ml PBS/T+0.5% BSA per assay plate. Sheep- anti-mouse IgG HRP-conjugated secondary antibody from Amersham Pharmacia Biotech UK Ltd. (NXA931). Dilute 20 μl of stock into 11 ml PBS/T+0.5% BSA per assay plate.

### TMB Solution

Dissolve 1 mg TMB tablet (Sigma T5525) into 1 ml DMSO (Sigma, D8779) in the dark for 1 hour at room temperature. Add this solution to 9 ml of freshly prepared 50 mM phosphate-citrate buffer pH 5.0+0.03% sodium perborate [1 buffer capsule (Sigma P4922) per 100 ml distilled water].

Stop solution is 1M H₂SO₄ (Fisher Scientific UK. Cat. No. S/9200/PB08).

### Test Compound

Dissolve in DMSO to 10 mM then dilutions in distilled water to give a range from 200 to 0.0026 μM in 1-2% DMSO final concentration in assay well.

### b.ii) Assay Protocol

The poly EAY substrate was diluted to 1 μg/ml in PBS and then dispensed in an amount of 100 μl per well into a 96-well plate. The plate was sealed and incubated overnight at 4° C. Excess poly EAY solution was discarded and the plate was washed (2× PBS/T; 250 μl PBS per well), blotting dry between washes. The plate was then washed again (1×50 mM HEPES, pH 7.4; 250 μl per well) and blotted dry (this is important in order to remove background phosphate levels). 10 μl test compound solution was added with 40 μl of kinase solution to each well. Then 50 μl of co-factor solution were added to each well and the plate was incubated for 60 minutes at room temperature.

The plate was emptied (i.e. the contents were discarded) and was washed twice with PBS/T (250 μl per well), blotting dry between each wash. 100 μl of diluted anti-phosphotyrosine antibody were added per well and the plate was incubated for 60 minutes at room temperature.

The plate was again emptied and washed twice with PBS/T (250 μl per well), blotting dry between each wash. 100 μl of diluted sheep- anti-mouse IgG antibody were added per well and the plate was left for 60 minutes at room temperature. The contents were discarded and the plate washed twice with PBS/T (250 μl per well), blotting dry between each wash. 100 μl of TMB solution were added per well and the plate was incubated for 5-10 minutes at room temperature (solution turns blue in the presence horse radish peroxidase).

Reaction was stopped with 50 μl of H₂SO₄ per well (turns the blue solution yellow) and the plate was read at 450 nm in Versamax plate reader (Molecular Devices Corporation, Calif., USA) or similar.

The compounds of the Examples were found to have an IC₅₀ in the above test of less than 100 μM.

c) Inhibition of IGF-Stimulated Cell Proliferation

The construction of murine fibroblasts (NIH3T3) over-expressing human IGF-1 receptor has been described by Lammers et al (EMBO J, 8, 1369-1375, 1989). These cells show a proliferative response to IGF-I which can be measured by BrdU incorporation into newly synthesised DNA. Compound potency was determined as causing inhibition of the IGF-stimulated proliferation in the following assay:

### c.i) Reagents Used:

Cell Proliferation ELISA, BrdU (colorimetric) [Boehringer Mannheim (Diagnostics and Biochemicals) Ltd, UK. Cat no. 1 647 229]. DMEM, FCS, Glutamine, HBSS (all from Life Technologies Ltd., UK). Charcoal/Dextran Stripped FBS (HyClone SH30068.02, Perbio Science UK Ltd). BSA (Sigma, A7888).

Human recombinant IGF-1 Animal/media grade (GroPep Limited ABN 78 008 176 298, Australia. Cat No. IU 100).

### Preparation and Storage of IGF

100 μg of lyophilised IGF was reconstituted in 100 ul of 10 mM HCl.
Add 400 μl of 1 mg/ml BSA in PBS
25 μl aliquots@200 μg/ml IGF-1
Stored at −20° C.

### For Assay:

10 μl of stock IGF+12.5 ml growth medium to give 8× stock of 160 ng/ml.

### Complete Growth Medium

DMEM, 10% FCS, 2 mM glutamine.

### Starvation Medium

DMEM, 1% charcoal/dextran stripped FCS, 2 mM glutamine.

### Test Compound

Compounds are initially dissolved in DMSO to 10 mM, followed by dilutions in DMEM+1% FCS+glutamine to give a range from 100 to 0.0.45 μM in 1-0.00045% DMSO final concentration in assay well.

### c.ii) Assay Protocol

### Day 1

Exponentially growing NIH3T3/IGFR cells were harvested and seeded in complete growth medium into a flat-bottomed 96 well tissue culture grade plate (Costar 3525) at 1.2×10⁴ cells per well in a volume of 100 μl.

### Day 2

Growth medium was carefully removed from each well using a multi-channel pipette. Wells were carefully rinsed three times with 200 μl with HBSS. 100 μl of starvation medium was added to each well and the plate was re-incubated for 24 hours.

### Day 3

50 μl of a 4× concentrate of test compound was added to appropriate wells. Cells were incubated for 30 minutes with compound alone before the addition of IGF. For cells treated with IGF, an appropriate volume (i.e. 25 μl) of starvation medium was added to make a final volume per well up to 200 μl followed by 25 μl of IGF-1 at 160 ng/ml (to give a final concentration of 20 ng/ml). Control cells unstimulated with IGF also had an appropriate volume (i.e. 50 μl) of starvation medium added to make final volume per well up to 200 μl. The plate was re-incubated for 20 hours.

### Day 4

The incorporation of BrdU in the cells (after a 4 h incorporation period) was assessed using the BrdU Cell Proliferation Elisa according to the manufacturer's protocol.

The compounds of the Examples were found to have an IC₅₀ in the above test of less than 50 ρM.

### d) Mechanism of Action Assay

Inhibition of IGF-IR mediated signal transduction was determined by measuring changes in phosphorylation of IGF-IR, Akt and MAPK (ERK1 and 2) in response to IGF-I stimulation of MCF-7 cells (ATCC No. HTB-22). A measure of selectivity was provided by the effect on MAPK phosphorylation in response to EGF in the same cell line.

### d.i) Reagents Used:

RPMI 1640 medium, RPMI 1640 medium without Phenol Red, FCS, Glutamine (all from Life Technologies Ltd., UK).

Charcoal/Dextran Stripped FBS (HyClone SH30068.02, Perbio Science UK Ltd).

SDS (Sigma, L4390).

2-mercaptoethanol (Sigma, M6250).

Bromophenol blue (Sigma, B5525).

Ponceau S (Sigma, P3504).

Tris base (TRIZMA™ base, Sigma, T1503).

Glycine (Sigma, G7403).

Methanol (Fisher Scientific UK. Cat. No. M/3950/21).

Dried milk powder (Marvel™, Premier Brands UK Ltd.).

Human recombinant IGF-1 Animal/media grade (GroPep Limited ABN 78 008 176 298, Australia. Cat No. IU 100).

Human recombinant EGF (Promega Corporation, WI, USA. Cat. No. G5021).

### Complete Growth Medium

RPMI 1640, 10% FCS, 2mM glutamine

### Starvation Medium

RPMI1640 medium without Phenol Red, 1% charcoal/dextran stripped FCS, 2 mM glutamine.

### Test Compound

Compounds were initially dissolved in DMSO to 10 mM, followed by dilutions in RPMI 1640 medium without Phenol Red+1% FCS+2 mM glutamine to give a range from 100 to 0.0.45 μM in 1-0.00045% DMSO final concentration in assay well.

### Western Transfer Buffer

50 mM Tris base, 40 mM glycine, 0.04% SDS, 20% methanol.

### Laemmli Buffer x2:

100 mM Tris-HCl pH6.8, 20% glycerol, 4% SDS.

### Sample Buffer ×4:

200 mM 2-mercaptoethanol, 0.2% bromophenol blue in distilled water.

### Primary Antibodies

Rabbit anti-human IGF-1Rβ (Santa Cruz Biotechnology Inc., USA, Cat. No sc-713) Rabbit anti-insulin/IGF-1R [pYpY^{1162/1163}]Dual Phosphospecific (BioSource International Inc, CA, USA. Cat No. 44-8041).

Mouse anti-PKBα/Akt (Transduction Laboratories, KY, USA. Cat. No. P67220) Rabbit anti-Phospho-Akt (Ser473) (Cell Signalling Technology Inc, MA, USA. Cat. No.#9271).

Rabbit anti-p44/p42 MAP kinase (Cell Signalling Technology Inc, MA, USA. Cat. No.#9102).

Rabbit anti-Phospho p44/p42 MAP kinase (Cell Signalling Technology Inc, MA, USA. Cat. No.#9101).

Mouse anti-actin clone AC-40 (Sigma-Aldrich Company Ltd, UK, A4700).

### Antibody Dilutions

| | | Secondary antibody in |
|---|---|---|
| Antibody | Dilution in PBST | PBST |
| IGFR | 1:200 with 5% milk | Anti-rabbit with 5% milk |
| Phospho-IGFR | 1:1000 with 5% milk | Anti-rabbit with 5% milk |
| Akt | 1:1000 with 5% milk | Anti-mouse with 5% milk |
| PhosphoAkt | 1:1000 with 5% milk | Anti-rabbit with 5% milk |
| MAPK | 1:1000 with 5% milk | Anti-rabbit with 5% milk |
| Phospho-MAPK | 1:1000 with 5% milk | Anti-rabbit with 5% milk |
| Actin | 1:1000 with 5% milk | Anti-mouse with 5% milk |

### Secondary Antibodies

Goat anti-rabbit, HRP linked (Cell Signalling Technology Inc, MA, USA. Cat. No.#7074).

Sheep- anti-mouse IgG HRP-conjugated (Amersham Pharmacia Biotech UK Ltd. Cat. No. NXA931).

Dilute anti-rabbit to 1:2000 in PBST+5% milk.

Dilute anti-mouse to 1:5000 in PBST+5% milk.

### d.ii) Assay Protocol

### Cell Treatment

MCF-7 cells were plated out in a 24 well plate at 1×10⁵ cells/well in 1 ml complete growth medium. The plate was incubated for 24 hours to allow the cells to settle. The medium was removed and the plate was washed gently 3 times with PBS 2 ml/well. 1 ml of starvation medium was added to each well and the plate was incubated for 24 hours to serum starve the cells.

Then 25 μl of each compound dilution was added and the cells and compound were incubated for 30 minutes at 37° C. After 30 minutes incubation of the compound, 25 μl of IGF (for 20 ng/ml final concentration) or EGF (for 0.1 ng/ml final concentration) was added to each well as appropriate and the cells incubated with the IGF or EGF for 5 minutes at 37° C. The medium was removed (by pipetting) and then 100 μl of 2× Laemmli buffer was added. The plates were stored at 4° C. until the cells were harvested. (Harvesting should occur within 2 hours following addition of Laemmli buffer to the cells.) To harvest the cells, a pipette was used to repeatedly draw up and expel the Laemmli buffer/cell mix and transfer into a 1.5 ml Eppendorf tube. The harvested cell lysates were kept at −20° C. until required. The protein concentration of each lysate could be determined using the DC protein assay kit (Bio-Rad Laboratories, USA, according to manufacturer's instructions).

### Western Blot Technique

Cell samples were made up with 4× sample buffer, syringed with a 21 gauge needle and boiled for 5 minutes. Samples were loaded at equal volumes and a molecular weight ladder on 4-12% Bis-Tris gels (Invitrogen BV, The Netherlands) and the gels were run in an Xcell SureLock™ Mini-Cell apparatus (Invitrogen) with the solutions provided and according to the manufacturer's instructions. The gels were blotted onto Hybond C Extra™ membrane (Amersham Pharmacia Biotech UK Ltd.) for 1 hour at 30 volts in the Xcell SureLock™ Mini-Cell apparatus, using Western transfer buffer. The blotted membranes were stained with 0.1% Ponceau S to visualise transferred proteins and then cut into strips horizontally for multiple antibody incubations according to the molecular weight standards. Separate strips were used for detection of IGF-1R, Akt, MAPK and actin control.

The membranes were blocked for 1 hour at room temperature in PBST+5% milk solution. The membranes were then placed into 3 ml primary antibody solution in 4 well plates and the plates were incubated overnight at 4° C. The membranes were washed in 5 ml PBST, 3 times for 5 minutes each wash. The HRP-conjugated secondary antibody solution was prepared and 5 ml was added per membrane. The membranes were incubated for 1 hour at room temperature with agitation. The membranes were washed in 5 ml PBST, 3 times for 5 minutes each wash. The ECL solution (SuperSignal ECL, Pierce, Perbio Science UK Ltd) was prepared and incubated with the membranes for 1 minute (according to manufacturer's instructions), followed by exposure to light sensitive film and development.

The compounds of the Examples were found to have an IC₅₀ in the above test of less than 20 μM.

By way of example, the following Table illustrates the activity of representative compounds according to the invention. Column 2 of the Table shows IC₅₀ data from Test (c) described above for the inhibition of IGF-stimulated proliferation in murine fibroblasts (NIH3T3) over-expressing human IGF-1 receptor:

| Example Number | IC₅₀ (μM) - Test (c) |
|---|---|
| 2 | 0.04 |
| 4 | 0.06 |

We have found that the compounds of the present invention possess anti-proliferative properties such as anti-cancer properties that are believed to arise from their IGF-1R tyrosine kinase inhibitory activity. Furthermore, certain of the compounds according to the present invention possess substantially better potency against the IGF-1R tyrosine kinase than against other tyrosine kinases enzymes. Such compounds possess sufficient potency against the IGF-1R tyrosine kinase that they may be used in an amount sufficient to inhibit IGF-1R tyrosine kinase whilst demonstrating little, or significantly lower, activity against other tyrosine kinases. Such compounds are likely to be useful for the effective treatment of, for example, IGF-1R driven tumours.

Accordingly, the compounds of the present invention are expected to be useful in the treatment of diseases or medical conditions mediated alone or in part by IGF-1R tyrosine kinase, i.e. the compounds may be used to produce an IGF-1R tyrosine kinase modulatory or inhibitory effect in a warm-blooded animal in need of such treatment. Thus the compounds of the present invention provide a method for the treatment of malignant cells characterised by modulation or inhibition of the IGF-1R tyrosine kinase. Particularly the compounds of the invention may be used to produce an anti-proliferative and/or pro-apoptotic and/or anti-invasive effect mediated alone or in part by the modulation or inhibition of IGF-1R tyrosine kinase. Particularly, the compounds of the present invention are expected to be useful in the prevention or treatment of those tumours that are sensitive to modulation or inhibition of IGF-1R tyrosine kinase that is involved in the signal transduction steps which drive proliferation and survival of these tumour cells. Accordingly the compounds of the present invention are expected to be useful in the treatment and/or prevention of a number of proliferative and hyperproliferative diseases/conditions, examples of which include the following cancers:
(1) carcinoma, including that of the bladder, brain, breast, colon, kidney, liver, lung, ovary, pancreas, prostate, stomach, cervix, colon, thyroid and skin;
(2) hematopoietic tumours of lymphoid lineage, including acute lymphocytic leukaemia, B-cell lymphoma and Burketts lymphoma;
(3) hematopoietic tumours of myeloid lineage, including acute and chronic myelogenous leukaemias, promyelocytic leukaemia and multiple myeloma;
(4) tumours of mesenchymal origin, including fibrosarcoma and rhabdomyosarcoma; and
(5) other tumours, including melanoma, seminoma, tetratocarcinoma, neuroblastoma and glioma.

The compounds of the invention are expected to be especially useful in the treatment of tumours of the breast, colon and prostate and in the treatment of multiple myeloma.

According to this aspect of the invention there is provided a compound of formula (I), or a pharmaceutically-acceptable salt thereof, for use as a medicament.

Thus according to this aspect of the invention there is provided the use of a compound of formula (I), or a pharmaceutically-acceptable salt thereof, as defined hereinbefore in the manufacture of a medicament for use in the production of an anti-proliferative effect in a warm-blooded animal such as man.

According to a further feature of this aspect of the invention there is provided a method for producing an anti-proliferative effect in a warm-blooded animal, such as man, in need of such treatment which comprises administering to said animal an effective amount of a compound of formula (I), or a pharmaceutically-acceptable salt thereof, as hereinbefore defined.

According to a further aspect of the invention there is provided a compound of formula (I), or a pharmaceutically-acceptable salt thereof, for use in the production of an anti-proliferative effect in a warm-blooded animal such as man.

According to a further aspect of the invention there is provided the use of a compound of formula (I), or a pharmaceutically-acceptable salt thereof, as defined hereinbefore in the manufacture of a medicament for use in the production of an anti-proliferative effect which effect is produced alone or in part by inhibiting IGF-1R tyrosine kinase in a warm-blooded animal such as man.

According to a further feature of this aspect of the invention there is provided a method for producing an anti-proliferative effect which effect is produced alone or in part by inhibiting IGF-1R tyrosine kinase in a warm-blooded animal, such as man, in need of such treatment which comprises administering to said animal an effective amount of a compound of formula (I), or a pharmaceutically-acceptable salt thereof, as hereinbefore defined.

According to a further aspect of the invention there is provided a compound of formula (I), or a pharmaceutically-acceptable salt thereof, for use in the production of an anti-proliferative effect which effect is produced alone or in part by inhibiting IGF-1R tyrosine kinase in a warm-blooded animal such as man.

According to a further aspect of the present invention there is provided the use of a compound of formula (I), or a pharmaceutically-acceptable salt thereof, as defined hereinbefore in the manufacture of a medicament for use in the treatment of a disease or medical condition (for example a cancer as mentioned herein) mediated alone or in part by IGF-1R tyrosine kinase.

According to a further feature of this aspect of the invention there is provided a method for treating a disease or medical condition (for example a cancer as mentioned herein) mediated alone or in part by IGF-1R tyrosine kinase in a warm-blooded animal, such as man, in need of such treatment, which comprises administering to said animal an effective amount of a compound of formula (I), or a pharmaceutically-acceptable salt thereof, as defined hereinbefore.

According to a further aspect of the invention there is provided a compound of formula (I), or a pharmaceutically-acceptable salt thereof, for use in the treatment of a disease or medical condition (for example a cancer as mentioned herein) mediated alone or in part by IGF-1R tyrosine kinase.

According to a further aspect of the invention there is provided the use of a compound of formula (I), or a pharmaceutically-acceptable salt thereof, as defined hereinbefore in the manufacture of a medicament for use in the prevention or treatment of those tumours which are sensitive to inhibition of IGF-1R tyrosine kinase involved in the signal transduction steps which lead to the proliferation of tumour cells.

According to a further feature of this aspect of the invention there is provided a method for the prevention or treatment of those tumours which are sensitive to inhibition of IGF-1R tyrosine kinase, involved in the signal transduction steps which lead to the proliferation and/or survival of tumour cells in a warm-blooded animal, such as man, in need of such treatment, which comprises administering to said animal an effective amount of a compound of formula (I), or a pharmaceutically-acceptable salt thereof, as defined hereinbefore.

According to a further aspect of the invention there is provided a compound of formula (I), or a pharmaceutically-acceptable salt thereof, for use in the prevention or treatment of those tumours which are sensitive to inhibition of IGF-1R tyrosine kinase, involved in the signal transduction steps which lead to the proliferation and/or survival of tumour cells.

According to a further aspect of the invention there is provided the use of a compound of formula (I), or a pharmaceutically-acceptable salt thereof, as defined hereinbefore in the manufacture of a medicament for use in providing an IGF-1R tyrosine kinase inhibitory effect.

According to a further feature of this aspect of the invention there is provided a method for providing an IGF-1R tyrosine kinase inhibitory effect in a warm-blooded animal, such as man, in need of such treatment, which comprises administering to said animal an effective amount of a compound of formula (I), or a pharmaceutically-acceptable salt thereof, as defined hereinbefore.

According to a further aspect of the invention there is provided a compound of formula (I), or a pharmaceutically-acceptable salt thereof, for use in providing an IGF-1R tyrosine kinase inhibitory effect.

According to a further aspect of the present invention there is provided the use of a compound of formula (I), or a pharmaceutically-acceptable salt thereof, as defined hereinbefore in the manufacture of a medicament for use in the treatment of a cancer, for example a cancer selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer.

According to a further feature of this aspect of the invention there is provided a method for treating a cancer, for example a cancer selected from selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer in a warm-blooded animal, such as man, in need of such treatment, which comprises administering to said animal an effective amount of a compound of formula (I), or a pharmaceutically-acceptable salt thereof, as defined hereinbefore.

According to a further aspect of the invention there is provided a compound of formula (I), or a pharmaceutically-acceptable salt thereof, for use in the treatment of a cancer, for example a cancer selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer.

As mentioned above the size of the dose required for the therapeutic or prophlyactic treatment of a particular disease will necessarily be varied depending upon, amongst other things, the host treated, the route of administration and the severity of the illness being treated.

The compounds of the invention may be administered in the form of a pro-drug, by which we mean a compound that is broken down in a warm-blooded animal, such as man, to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxylic acid or a hydroxy group in a compound of formula (I).

Accordingly, the present invention includes those compounds of formula (I) as defined hereinbefore when made available by organic synthesis and when made available within the human or animal body by way of cleavage of a pro-drug thereof. Accordingly, the present invention includes those compounds of formula (I) that are produced by organic synthetic means and also such compounds that are produced in the human or animal body by way of metabolism of a precursor compound, that is a compound of formula (I) may be a synthetically-produced compound or a metabolically-produced compound.

A suitable pharmaceutically-acceptable pro-drug of a compound of formula (I) is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309 to 396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, edited by H. Bundgaard, p. 113 to 191 (1991);
d) H. Bundgaard, Advanced Drug Delivery Reviews, 8, 1 to 38 (1992); and
e) H. Bundgaard, et al., Journal of Pharmaceutical Sciences, 77, 285 (1988).

The compounds of formula (I), and pharmaceutically-acceptable salts thereof, may be used on their own but will generally be administered in the form of a pharmaceutical composition in which the formula (I) compound/salt (active ingredient) is in association with a pharmaceutically-acceptable adjuvant, diluent or carrier.

Thus, the present invention also provides a pharmaceutical composition comprising a compound of formula (I), or a pharmaceutically-acceptable salt thereof, as hereinbefore defined, in association with a pharmaceutically-acceptable adjuvant, diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The invention further provides a process for the preparation of a pharmaceutical composition of the invention which comprises mixing a compound of formula (I), or a pharmaceutically-acceptable salt thereof, as hereinbefore defined, with a pharmaceutically-acceptable adjuvant, diluent or carrier.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of formula (I) will naturally vary according to the nature and severity of the conditions, the age and sex of the animal or patient and the route of administration, according to well known principles of medicine.

In using a compound of formula (I) for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms will contain about 0.5 mg to 0.5 g of a compound of this invention.

The anti-proliferative treatment defined hereinbefore may be applied as a sole therapy or may involve, in addition to the compound of the invention, conventional surgery or radiotherapy or chemotherapy. Such chemotherapy may include one or more of the following categories of anti-tumour agents:
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™]and the anti-erbB1 antibody cetuximab [Erbitux, C225]); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD 1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications W097/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a compound of formula (I), or a pharmaceutically-acceptable salt thereof, as defined hereinbefore and an additional anti-tumour agent as defined hereinbefore for the conjoint treatment of cancer.

Although the compounds of formula (I) are primarily of value as therapeutic agents for use in warm-blooded animals (including man), they are also useful whenever it is required to inhibit the effects of IGF-1R tyrosine kinases. Thus, they are useful as pharmacological standards for use in the development of new biological tests and in the search for new pharmacological agents.

### EXAMPLES

The invention will now be further described with reference to the following illustrative examples—in which, unless stated otherwise:
(i) temperatures are given in degrees Celsius (° C); operations were carried out at room or ambient temperature, that is, at a temperature in the range of 18 to 25° C.;
(ii) organic solutions were dried over anhydrous magnesium sulfate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz, in DMSO-d₆ unless otherwise indicated. The following abbreviations have been used: s, singlet; d, doublet; t, triplet; q, quartet; m, multiplet; br, broad. Where NMR spectra are broad (due to hindered rotation or slow proton exchange), NMR spectra were run at 100° C.;
(viii) chemical symbols have their usual meanings; SI units and symbols are used;
(ix) solvent ratios are given in volume:volume (v/v) terms; and
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(xi) the following abbreviations have been used:

THF tetrahydrofuran;

EtOAc ethyl acetate;

DCM dichloromethane;

DMSO dimethylsulfoxide;

DIPEA diisopropylethylamine;

NMP N-methylpyrrolid-2-one;

tBuOH tert-butyl alcohol;

TFA trifluoroacetic acid;

DMF N,N-dimethylformamide; and

DMA N,N-dimethylacetamide.

### Example 1

### 6-Chloro-2-{2-[3-(pyrid-2-yl)isoxazol-5-yl]azetidin-1-yl}-4-(5-methyl-1H-pyrazol-3-ylamino)pyrimidine

A mixture of 2,6-dichloro-4-(5-methyl-1H-pyrazol-3-ylamino)pyrimidine (601.9 mg, 2.46 mmol), diisopropylethylamine (383.3 mg, 2.96 mmol) and 2-(3-[2-pyridyl]isoxazol-5-yl)azetidine (595 mg, 2.96 mmol) in n-butanol (20 ml) was heated under a nitrogen atmosphere at 70° C. for 18 hours. The solvent was removed by evaporation and the residue purified by chromatography on silica gel eluting with DCM and then methanol/DCM (2:98) to give the title compound (876 mg, 87.2%) as a foam; Mass Spectrum 409 [MH]+.

The 2,6-dichloro-4-(5-methyl-1H-pyrazol-3-ylamino)pyrimidine starting material was prepared as follows:

A mixture of 2,4,6-trichloropyrimidine (1.0 g, 5.4 mmol), 3-amino-5-methyl-1H-pyrrazole (0.53 g, 5.4 mmol) and sodium carbonate (0.57 g, 5.4 mmol) in ethanol (25 ml) was stirred at ambient temperature for 18 hours. Water was added and the resulting precipitate was collected by filtration, washed with water and a small amount of methanol and dried to give 2,6-dichloro-4-(5-methyl-1H-pyrazol-3-ylamino)pyrimidine (1.15 g, 88%) as a colourless crystalline solid; NMR Spectrum 2.23 (s, 3H), 6.01 (s, 1H), 7.24 (s, 1H), 10.25 (br s, 1H), 11.9 (br s, 1H); Mass Spectrum 244 [MH]+.

The 2-(3-[2-pyridyl]isoxazol-5-yl)azetidine starting material was prepared as follows:

A mixture of (S)-2-azetidinecarboxyic acid (10 g, 98.9 mmol), di-tert butyl dicarbonate (28.06 g, 128.6 mmol), N-methyl molpholine (11.5 g, 113.7 mmol), 1,4-dioxane (160 ml) and water (160 ml) was stirred at 0° C, for 4 hours and then at ambient temperature for 18 hours. The volatiles were removed by evaporation and the residue was dissolved in water, washed with DCM and the aqueous layer acidified to pH 1.0 at 0° C. with concentrated hydrochloric acid. The aqueous layer was extracted with DCM and the organic layer dried (Na₂SO₄) over anhydrous sodium sulfate to give N-tert-butyloxycarbonylazetidin-2-yl carboxylic acid (12.61 g, 63.1%) as an oil; NMR Spectrum (CDCl₃) 1.42 (s, 9H), 2.40 (m, 1H), 2.59 (m, 1H), 3.90 (m, 2H), 4.80 (t, 1H).

Isobutyl chloroformate (8.431 g, 61.7 mmol) was added to a solution of N-tert-butyloxycarbonylazetidin-2-yl carboxylic acid (12.42 g, 61.7 mmol) and N-methyl morpholine (6.23 g, 61.7 mmol) in anhydrous THF (20 ml) cooled at −10° C. under an nitrogen atmosphere, at such a rate to keep the reaction temperature below −8 ° C. The reaction mixture was then stirred at −8° C. for 1 hour and then at ambient temperature for a further 3 hours. Methanol (200 ml) was added and the reaction mixture stirred at ambient temperature for 18 hours. The volatiles were removed by evaporation and the residue partitioned between water and diethylether. The organic layer was separated, dried (Na₂SO₄) give methyl N-tert-butyloxycarbonylazetidin-2-yl carboxylate (11.15 g, 84.0%) as an oil; NMR Spectrum (CDCl₃) 1.43 (s, 9H), 2.20 (m, 1H), 2.50 (m, 1H), 3.78 (s, 3H), 3.90 (m, 1H), 4.00 (m, 1H), 4.60 (m, 1H). 2-Acetylpyridyl oxime (11.63 g, 85.5 mmol) dissolved in anhydrous THF (100 ml) was added to a solution of 1.6M solution of n-butyllithium in hexanes (106 ml, 171 mmol) in anhydrous THF (50 ml) cooled in ice under an inert atmosphere, at such a rate to maintain reaction temperature below 0° C. The mixture was then stirred at 0° C. for 1 hour and a solution of methyl N-tert-butyloxycarbonylazetidin-2-yl carboxylate (5.575 g, 25.9 mmol) in anhydrous THF (50 ml) was added at such a rate as to maintain reaction temperature below 0° C. The mixture was then stirred at 0° C. for 3.5 hours, then at ambient temperature for 18 hours. The reaction mixture was quenched at 0° C. with 2N sodium hydroxide aqueous solution and then extracted with DCM. The solvent was removed and the residue purified by chromatography on silica gel eluting with EtOAc /hexane (2:3) and then with EtOAc to give the N-(tert-butyloxycarbonyl)-2-(3-[2-pyridyl]-4,5-dihydro-5-hydroxyisoxazol-5-yl)azetidine (4.42 g, 53.5%) as a oil; NMR Spectrum (CDCl₃) 2.48 (s, 9H), 2.03 (m, 1H), 2.20 (m, 1H), 3.18 (d, 1H), 3.40 (d, 1H), 3.90 (m, 2H), 4.75 (t, 1H), 7.27 (m, 1H), 7.55 (br s, 1H), 7.70 (t, 1H), 8.02 (d, 1H), 8.58 (m, 1H)

A mixture of N-(tert-butyloxycarbonyl)-2-(3-[2-pyridyl]-4,5-dihydro-5-hydroxyisoxazol-5-yl)azetidine (4.40 g, 13.7 mmol), potassium hydroxide (2.1 g, 12.2 mmol) and ethanol (100 ml) was heated at 100° C. for 2 hours. Volatiles were removed by evaporation, the residue dissolved in DCM and the insoluble material removed by filtration. The solvent was removed from the filtrate by evaporation to give N-(tert-butyloxycarbonyl)-2-(3-[2-pyridyl]-isoxazol-5-yl)azetidine (4.12g, 100%) as a solid; NMR Spectrum (CDCl₃) 1.38 (s, 9H), 2.45 (m, 1H), 2.65 (m, 1H), 4.02 (m, 2H), 5.35 (m, 1H), 6.90 (s, 1H), 7.35 (m, 1H), 7.80 (t, 1H), 8.05 (d, 1H), 8.68 (d, 1H); Mass Spectrum 302 [MH]+.

A mixture of 1-(tert-butyloxycarbonyl)-2-(3-[2-pyridyl]-isoxazol-5-yl)azetidine (4.12 g, 13.7 mmol), 4N hydrogen chloride solution in 1,4-dioxane (40 ml, 160 mmol) and methanol (50 ml) was heated at 50° C. for 2 hours. The mixture allowed to cool and poured on to a 50 g isolute SCX-2 ion exchange column. The column was eluted with methanol to elute any neutrals, followed by 7M methanolic ammonia to elute the product. The solvent was removed by evaporation to give 2-(3-[2-pyridyl]isoxazol-5-yl)azetidine (2.33g, 85.0%) as an oil; NMR Spectrum (CDCl₃) 2.70 (m, 2H), 3.60 (m, 1H), 3.75 (m, 1H), 5.08 (t, 1H), 6.90 (s, 1H), 7.37 (m, 1H), 7.80 (t, 1H), 8.07 (d, 1H), 8.70 (d, 1H); Mass Spectrum 202 [MH]+.

### Example 2

### 6-Morpholino-2-{2-[3-(pyrid-2-yl)isoxazol-5-yl]azetidin-1-yl}-4-(5-methyl-1H-pyrazol-3-ylamino)pyrimidine

A mixture of 6-chloro-2-{2-[3-(pyrid-2-yl)isoxazol-5-yl]azetidin-1-yl}-4-(5-methyl-1H-pyrazol-3-ylamino)pyrimidine (876 mg, 2.14 mmol) and morpholine (3 g, 344 mmol) was heated at 120° C. for 1 hour under a nitrogen atmosphere. The mixture allowed to cool and the volatiles removed by evaporation. The residue was triturated with water and diethylether. The resulting solid product was collected by filtration, dissolved in DCM and purified by chromatography on silica gel eluting with methanol/diethylether/aqueous ammonia (5:94:1) to give the title compound as solid after evaporation (278 mg, 28.3%); NMR Spectrum (DMSO-d₆+d₄-acetic acid at 100° C.) 2.10 (s, 3H), 2.50 (m, 3H), 2.70 (m, 1H), 3.35 (m, 2H), 3.55 (m, 1H), 4.05 (m, 2H), 5.43 (t, 1H), 5.91 (s, 1H), 6.93 (s, 1H), 7.45 (s, 1H), 7.88 (t, 1H), 7.97 (d, 1H), 8.68 (d, 1H); Mass Spectrum 460 [MH]+.

### Example 3

### 6-Methoxy-2-{2-[3-(pyrid-2-yl)isoxazol-5-yl]azetidin-1-yl}-4-(5-methyl-1H-pyrazol-3-ylamino)pyrimidine

6-Chloro-2-{2-[3-(pyrid-2-yl)isoxazol-5-yl]azetidin-1-yl}-4-(5-methyl-1H-pyrazol-3-ylamino)pyrimidine (154 mg, 0.38 mmol) was added to a 25% solution of sodium methoxide in methanol (4 ml) and the mixture heated at 120° C. for 20 minutes under microwave irradiation. The volatiles were removed by evaporation and water (50 ml) was added to the residue. The resulting precipitated product was collected by filtration and purified by reverse phase HPLC using a C8 column eluting with water/acetonitrile/TFA (95:5:0.2 decreasing in polarity to 0:100:0.2). The fractions containing product were combined and passed through a 10 g isolute SCX-2 ion exchange column. The column was eluted with methanol to remove neutral impurities and then with 2M methanolic ammonia to elute the product. The solvent was removed by evaporation to give the title compound (57 mg, 37%) as a cream powder; NMR Spectrum (DMSO-d₆+d₄-acetic acid at 100° C.) 2.10 (s, 3H), 2.50 (m, 1H), 2.75 (m, 1H), 4.05-4.20 (m, 2H), 5.50 (dd, 1H), 5.95 (s, 1H), 6.95 (s, 1H), 7.45 (m, 1H), 7.85 (m, 1H), 7.95 (d, 1H), 8.65 (d, 1H); Mass Spectrum 405 [MH]+.

### Example 4

### 6-Methyl-2-{2-[3-(pyrid-2-yl)isoxazol-5-yl]azetidin-1-yl}-4-(5-methyl-1H-pyrazol-3-ylamino)pyrimidine

A mixture of 2-chloro-6-methyl-4-(5-methyl-1H-pyrazol-3-ylamino)pyrimidine (200 mg, 0.9 mmol), 2-(3-[2-pyridyl]isoxazol-5-yl)azetidine (191 mg, 0.95 mmol), and diisopropylethylamine (0.32 ml, 1.80 mmol) in hexanol (3 ml) were heated at 130° C. for 30 minutes. The crude reaction mixture was allowed to cool and purified directly by reverse phase HPLC using a C18 column eluting with water/acetonitrile/TFA (95:5:0.2 decreasing in polarity to 0: 100:0.2). The fractions containing product were combined and passed through an isolute SCX-2 ion exchange column. The column was eluted with methanol to elute any neutrals, followed by 7M methanolic ammonia to elute the product. The solvent was removed by evaporation to give the title compound (69 mg, 20 %) as a white solid; NMR Spectrum 2.10 (s, 3H), 2.14 (s, 3H), 2.50 (m, 1H), 2.76 (m, 1H), 4.09 (m, 2H), 5.49 (dd, 1H), 600 (s, 1H), 6.27 (s, 1H), 6.95 (s, 1H), 7.47 (t, 1H), 7.92 (m, 1H), 8.0 (d, 1H), 8.68 (d, 1H), 8.87 (br s, 1H); Mass Spectrum 389 [MH]+.

The 2-chloro-6-methyl-4-(5-methyl-1H-pyrazol-3-ylamino)pyrimidine starting material was prepared as follows:

Solid sodium carbonate (1.2 g, 11.3 mmol) was added to a solution of 2,4-dichloro-6-methylpyrimidine (1.7g, 10.3 mmol) and 5-amino-3-methyl-1H-pyrazole (1.0 g, 10.3 mmol) in dry ethanol (50 ml) and the mixture heated and stirred at 42° C. for 3 days. The mixture was allowed to cool, the insoluble material was removed by filtration and the filter pad washed with ethanol (10 ml). The volatiles were removed from the filtrate by evaporation, keeping the bath temperature below 40° C. The residue was immediately purified by chromatography on silica gel eluting with methanol/DCM (5:95 increasing in polarity to 20:80) to give 2-chloro-6-methyl-4-(5-methyl-1H-pyrazol-3-ylamino)pyrimidine (758 mg, 33%) as a white solid; NMR Spectrum (CDCl₃) 2.17 (s, 3H), 2.11 (s, 3H), 5.88 (br s, 1H), 7.85 (br s, 1H), 8.80 (br s, 1H); Mass Spectrum 224 [MH]+.

### Example 5

### S-6-Ethyl-2-{2-[3-(2-methylpyrid-3-yl)isoxazol-5-yl]azetidin-1-yl}-4-(5-methyl-1H-pyrazol-3-ylamino)pyrimidine

A mixture of 2-chloro-6-ethyl-4-(5-methyl-1H-pyrazol-3-ylamino)pyrimidine (146 mg, 0.62 mmol), S-2-[3-(2-methylpyrid-3-yl)isoxazol-5-yl]azetidine (200 mg, 0.93 mmol) and DIPEA (0.3 ml, 1.7 mmol) in 1-hexanol (4 ml) was heated at 130° C. for 3 hours. The mixture was allowed to cool and the volatiles removed by evaporation. The residue was purified by chromatography on silica gel eluting with EtOAc/hexanes (5:95 increasing in polarity to 50:50). The purified product was triturated with DCM/hexanes, collected by filtration and dried to give the title compound (148 mg, 58%); NMR Spectrum 1.15 (t, 3H), 2,10 (s, 3H), 2.38-2.48 (m, 2H), 2.50-2.55 (m, 1H), 2.60 (s, 3H), 2.71-2.80 (m, 1H), 4.02-4.15 (m, 2H), 5.50 (dd, 1H), 6.00 (s, 1H), 6.78 (s, 1H), 6.81 (s, 1H), 7.30 (dd, 1H), 7.90 (d, 1H), 8.53 (d, 1H), 8.89 (s, 1H), 11.5 (s, 1H); Mass Spectrum 417 [MH]+.

The 2-chloro-6-ethyl-4-(5-methyl-1H-pyrazol-3-ylamino)pyrimidine starting material was prepared as follows:

A mixture of 2,4-dichloro-6-ethylpyrimidine (4.0 g, 22.6 mmol), 3-amino-5-methylpyrazole (2.19 g, 22.6 mmol) and sodium carbonate (2.88 g, 27.1 mmol) in ethanol (100 ml) was heated at 40° C. for 4 days. Insoluble material was removed from the hot mixture by filtration and the solvent removed from the resulting filtrate. The residue was purified by chromatography on silica gel eluting with hexane/EtOAc (50:50 in creasing in polarity to 0:100). The purified product was triturated with ether to give 2-chloro-6-ethyl-4-(5-methyl-1H-pyrazol-3-ylamino)pyrimidine (667 mg, 12%) as a white solid; NMR Spectrum 1.18 (t, 3H), 2.21 (s, 3H), 2.55 (q, 2H), 6.03 (s, 1H), 6.95 (s, 1H); Mass Spectrum 238 [MH+.

The S-2-[3-(2-methylpyrid-3-yl)isoxazol-5-yl]azetidine starting material was prepared as follows:

A mixture of the 3-hydroxymethyl-2-methylpyridine (9.0 g, 73.1 mmol) and manganese (IV) dioxide (28.1 g, 322 mmol) in DCM (100 ml) was heated at reflux for two days. The insolubles were removed by filtration through diatomaceous earth and the filter pad was washed with methanol/DCM. The solvent was removed from the filtrate by evaporation to give 2-methylpyridine-3-carboxaldehyde (7.5 g, 85%) as an oil; NMR Spectrum 2.78 (s, 3H), 7.43 (dd, 1H), 8.15 (dd, 1H), 8.66 (dd, 1H).

A solution of hydroxylamine hydrochloride (3.16 g, 45.1 mmol) in water (15 ml) was added to a cooled solution of sodium hydroxide (2.46 g, 61.5 mmol) in water (15 ml). The resulting aqueous solution was added to a mixture of 2-methylpyridine-3-carboxaldehyde (5.0 g, 40.9mmol), water (44 ml), ethanol (44 ml) and ice (70 g). The mixture was stirred at ambient temperature for 18 hours and adjusted to pH 7 by the addition of 2M hydrochloric acid. The resulting precipitate was collected by filtration washed with water and dried to give 2-methylpyridine-3-carboxaldehyde oxime (3.5 g, 62%); NMR Spectrum 2.55 (s, 3H), 7.22 (dd, 1H), 7.94 (dd, 1H), 8.39 (s, 1H), 8.42 (dd, 1H).

Diisobutylaluminium hydride (55.4 ml of a 1M solution in hexanes, 55.4 mmol) was added to a solution of methyl N-tert-butyloxycarbonylazetidin-2-yl carboxylate (9.9 g, 46.1 mmol) in dry DCM (120 ml) at −78° C. The reaction mixture was allowed to warm to 0° C., and potassium carbonate (12.7 g, 92.2 mmol) and dimethyl (1-diazo-2-oxopropyl) phosphonate (10.0 g, 52.08 mmol) were added and the mixture stirred for 18 hours at ambient temperature. Saturated aqueous sodium potassium tartrate solution was added and the mixture extracted with DCM. The extracts were combined were dried (MgSO₄) and the solvent was removed by evaporation. The residue was purified by chromatography on silica gel eluting with EtOAc/hexanes (10:90) to give S—N-tertbutoxycarbonyl-2-ethynylazetidine (4.2 g, 50%) as a colourless oil, which gradually crystallised. NMR Spectrum (CDCl₃) 1.46 (s, 9H), 2.20-2.34 (m, 1H), 2.44-2.58 (m, 2H), 3.80-3.90 (q, 1H), 3.90-4.00 (q, 1H), 4.68-4.76 (broad t, 1H). Optical Rotation: −190.5 (c=2, methanol).

Sodium hypochlorite (11 ml of a 13% aqueous solution, 19.2 mmol), was added to a stirred mixture of 2-methylpyridine-3-carboxaldehdye oxime (1.5 g, 11 mmol) and S—N-tertbutoxycarbonyl-2-ethynylazetidine (2.2 g, 12.1 mmol) in DCM (60 ml) at 0° C. The mixture was allowed to warm to ambient temperature and stirred for 18 hours. The DCM layer was separated, dried (Na₂SO₄), and the solvent removed by evaporation. The residue was purified by chromatography on silica gel eluting with EtOAc/hexanes (25:75 increasing in polarity to 50:50) to give S—N-(tert-butoxycarbonyl)-2-[3-(2-methylpyrid-3-yl)isoxazol-5-yl]azetidine (1.4 g, 41%); Mass Spectrum 316 [MH]+.

A solution of 4M hydrogen chloride in dioxane (20 ml) was added to a solution of S—N-(tert-butoxycarbonyl)-2-[3-(2-methylpyrid-3-yl)isoxazol-5-yl]azetidine (1.5 g, 4.7 mmol) in methanol (20 ml) and the mixture stirred at ambient temperature for 18 hours. The volatiles were removed by evaporation and the residue dissolved in water, basified with aqueous ammonia and extracted with DCM. The extracts were combined, dried (Na₂SO₄) and the solvent removed by evaporation. The residue was purified by chromatography on silica gel eluting with methanol/DCM (4:96) to give S-2-[3-(2-methylpyrid-3-yl)isoxazol-5-yl]azetidine (700 mg, 68%); NMR Spectrum 2.48-2.53 (m, 2H), 2.65 (s, 3H), 3.36-3.41 (m, 1H), 1H), 3.61-3.69 (m, 1H), 4.99-5.05 (dd, 1H), 6.75 (s, 1H), 7.32 (dd, 1H), 7.80 (dd, 1H), 8.55 (dd, 1H).

### Example 6

### S-6-Methyl-2-{2-[3-(2-methylpyrid-3-yl)isoxazol-5-yl]azetidin-1-yl}-4-(5-methyl-1H-pyrazol-3-ylamino)pyrimidine

A mixture of 2-chloro-6-methyl-4-(5-methyl-1H-pyrazol-3-ylamino)pyrimidine (140 mg, 0.62 mmol), S-2-[3-(2-methylpyrid-3-yl)isoxazol-5-yl]azetidine (200 mg, 0.93 mmol) and DIPEA (0.3 ml, 1.7 mmol) in 1-hexanol (4 ml) was heated at 130° C. for 3 hours. The mixture was allowed to cool and the volatiles removed by evaporation. The residue was purified by chromatography on silica gel eluting with methanol/DCM (5:95) to give the title compound (124mg, 50%); NMR Spectrum 2.10 (s, 3H), 2.15 (s, 3H), 2.50-2.55 (m, 1H), 2.65 (s, 1H), 2.71-2.80 (m, 1H), 4.03-4.15 (m, 2H), 5.50 (dd, 1H), 6.00 (s, 1H), 6.78 (s, 1H), 6.80 (s, 1H), 7.30 (dd, 1H), 7.89 (d, 1H), 8.53 (d, 1H), 8.85 (s, 1H), 11.5 (s, 1H); Mass Spectrum 403 [MH]+.

### Example 7

### S-6-Chloro-2-{2-[3-(2-methylpyrid-3-yl)isoxazol-5-yl]azetidin-1-yl}-4-(5-methyl-1H-pyrazol-3-ylamino)pyrimidine

A mixture of 2,6-dichloro-4-(5-methyl-1H-pyrazol-3-ylamino)pyrimidine (prepared according to Method 29 of WO2005/040159) (610 mg, 2.5 mmol), S-2-[3-(2-methylpyrid-3-yl)isoxazol-5-yl]azetidine (600 mg, 2.8 mmol) and DIPEA (1.1 ml, 6.2 mmol) in 1-hexanol (10 ml) was heated at 75° C. for 18 hours. The 1-hexanol was removed by evaporation and the residue adsorbed onto silica and purified by chromatography on silica gel eluting with methanol/DCM (5:95 increasing in polarity to 10:90) to give the title compound (770 mg, 73%); NMR Spectrum 2.10 (s, 3H), 2.50-2.55 (m, 1H), 2.60 (s, 3H), 2.80-2.85 (m, 1H), 4.02-4.20 (m, 2H), 5.55 (dd, 1H), 6.00 (s, 1H), 6.48 (s, 1H), 6.89 (s, 1H), 7.31 (dd, 1H), 7.90 (d, 1H), 8.54 (d, 1H), 9.36 (s, 1H), 11.50 (s, 1H); Mass Spectrum 423 [MH]+.

### Example 8

### S-6-Morpholino-2-{2-[3-(2-methylpyrid-3-yl)isoxazol-5-yl]azetidin-1-yl}-4-(5-methyl-1H-pyrazol-3-ylamino)pyrimidine

A mixture of S-6-chloro-2-{2-[3-(2-methylpyrid-3-yl)isoxazol-5-yl]azetidin-1-yl}-4-(5-methyl-1H-pyrazol-3-ylamino)pyrimidine (300 mg, 0.71 mmol) and morpholine (4 ml) was heated at 120° C. under microwave irradiation for 2 hours. The mixture was allowed to cool, diluted with water and extracted with EtOAc. The extracts were combined, washed with water, dried (Na₂SO₄) and the solvent removed by evaporation. The residue was purified by chromatography on silica gel eluting with methanol/EtOAc (0:100 increasing in polarity to 5:95) to give the title compound (120 mg, 33%); NMR Spectrum 2.05 (s, 2H), 2.5-2.45 (m, 1H), 2.60 (s, 1H), 2.6-2.7 (m, 1H), 3.30-3.35 (m, 2H), 3.60 (s, 3H), 3.98-4.05 (m, 2H), 5.42 (dd, 1H), 5.95 (s, 1H), 6.95 (s, 1H), 7.35 (dd, 1H), 7.92 (d, 1H), 8.55 (d, 1H), 8.95 (s, 1H), 11.65 (s, 1H); Mass Spectrum 472 [MH]+.

### Example 9

### S-6-Methyl-2-{2-[3-(3-ethoxypyrazin-2-yl)isoxazol-5-yl]azetidin-1-yl}-4-(5-methyl-1H-pyrazol-3-ylamino)pyrimidine

A mixture of 2-chloro-6-methyl-4-(5-methyl-1H-pyrazol-3-ylamino)pyrimidine (130 mg, 0.58 mmol), S-2-[3-(3-ethoxypyrazin-2-yl)isoxazol-5-yl]azetidine (200 mg, 0.87 mmol) and DIPEA (0.3 ml, 1.7 mmol) in 1-hexanol (4 ml) was heated at 130° C. for 3 hours. The mixture was allowed to cool and the volatiles removed by evaporation. The residue was purified by chromatography on silica gel eluting with EtOAc/hexanes (10:90) to give the title compound (93mg, 37%); NMR Spectrum 1.39 (t, 3H), 2.10 (s, 1H), 2.15 (s, 3H), 2.45-2.55 (m, 1H), 2.72-2.80 (m, 1H), 4.03-4.15 (m, 2H), 4.49 (q, 2H), 5.50 (dd, 1H), 6.01 (s, 1H), 6.29 (s, 1H), 6.96 (s, 1H), 8.30 (dd, 2H), 8.82 (s, 1H), 11.48 (s, 1H); Mass Spectrum 434 [MH]+.

The S-2-[3-(3-ethoxypyrazin-2-yl)isoxazol-5-yl]azetidine starting material was prepared as follows:

A mixture of 3-ethoxy-2-methylpiperazine (13.8 g, 0.1 mole), selenium dioxide (22,2 g, 0.2 mole) and diatomeous earth (22 g) were heated at reflux in EtOAc (350 ml) for 3 days. Additional selenium dioxide (22 g, 0.2 mole) was added and heating continued for a further 7 days. The insolubles were removed by filtration through diatomeous earth and the filtrate concentrated by evaporation. The residue was purified by chromatography silica gel eluting with EtOAc/hexanes (10:90 increasing in polarity to 25:75) to give 3-ethoxypyrazine-2-carboxaldehyde (9.6 g, 63%) as an oil; NMR Spectrum (CDCl₃) 1.45-1.55 (t, 3H), 4.55-4.65 (q, 2H), 8.30-8.40 (q, 2H), 10.30 (s, 1H).

A mixture of 3-ethoxypyrazine-2-carboxaldehyde (3.04 g, 20 mmol) and hydroxylamine hydrochloride (1.6 g, 0.23 mmol) in a mixture of ethanol (50 ml) and water (5 ml) was heated at reflux for 4 hours. The solution was allowed to cool and basified by careful addition of sodium hydrogen carbonate. Ethanol was removed by evaporation and water was added to the residue. The resulting precipitate was collected by filtration, washed with water and acetone, and dried to give 3-ethoxypyrazine-2-carboxaldehyde oxime (1.47 g, 44%); NMR Spectrum 1.30-1.40 (t, 3H), 4.35-4.50 (q, 2H), 8.2-8.3 (m, 2H), 11.9 (s, 1H).

Sodium hypochlorite (8.5 ml of a 13% aqueous solution, 14.8 mmol), was added to a stirred mixture of 3-ethoxypyrazine-2-carboxaldehyde oxime (1.32 g, 7.9 mmol) and S—N-tertbutoxycarbonyl-2-ethynylazetidine (1.72 g, 9.5 mmol) in DCM (60 ml) at 0° C. The mixture was allowed to warm to ambient temperature and stirred for 18 hours. The DCM layer was separated, dried (Na₂SO₄), and the solvent removed by evaporation. The residue was purified by chromatography on silica gel eluting with EtOAc/hexanes (25:75 increasing in polarity to 50:50) to give S—N-(tert-butoxycarbonyl)-2-[3-(3-ethoxypyrazin-2-yl)isoxazol-5-yl]azetidine (680 mg, 25%); NMR Spectrum 1.35 (s, 9H), 1.38 (t, 3H), 2.35-2.42 (m, 1H), 2.62-2.72 (m, 1H), 3.9-4.0 (m, 2H), 4.52 (q, 2H), 5.4 (dd, 1H), 6.90 (s, 1H), 8.32 (dd, 2H); Mass Spectrum 347 [MH]+.

A solution of 4M hydrogen chloride in dioxane (15 ml) was added to a solution of S—N-(tert-butoxycarbonyl)-2-[3-(3-ethoxypyrazin-2-yl)isoxazol-5-yl]azetidine (600 mg, 1.7 mmol) in methanol (15 ml) and the mixture stirred at ambient temperature for 18 hours. The volatiles were removed by evaporation and the residue dissolved in water, basified with aqueous ammonia and extracted with DCM. The extracts were combined, dried (Na₂SO₄) and the solvent removed by evaporation. The residue was purified by chromatography on silica gel eluting with methanol/DCM (4:96) to give S-2-[3-(3-ethoxypyrazin-2-yl)isoxazol-5-yl]azetidine (465 mg, 100%); NMR Spectrum 1.38 (t, 3H), 2.42-2.50 (m, 1H), 2.5-2.6 (m, 1H), 3.28-3.32 (m, 1H), 3.65-3.66 (m, 1H), 4.48 (q, 2H), 5.0 (dd, 1H), 6.88 (s, 1H), 8.85 (s, 2H).

### Example 10

### S-6-Ethyl-2-{2-[3-(3-ethoxyrazin-2-yl)isoxazol-5-yl]azetidin-1-yl}-4-(5-methyl-1H-pyrazol-3-ylamino)pyrimidine

A mixture of 2-chloro-6-ethyl-4-(5-methyl-1H-pyrazol-3-ylamino)pyrimidine (137 mg, 0.58 mmol), S-2-[3-(3-ethoxypyrazin-2-yl)isoxazol-5-yl]azetidine (200 mg, 0.87 mmol) and DIPEA (0.3 ml, 1.7 mmol) in 1-hexanol (4 ml) was heated at 130° C. for 3 hours. The mixture was allowed to cool and the volatiles removed by evaporation. The residue was purified by chromatography on silica gel eluting with EtOAc/hexanes (50:50 increasing in polarity to 100:0) to give the title compound (93 mg, 36%); NMR Spectrum 1.15 (t, 3H), 1.38 (t, 3H), 2.12 (s, 3H), 2.42 (q, 2H), 2.47-2.55 (m, 1H), 2.80-2.70 (m, 1H), 4.15-4.02 (m, 2H), 4.50 (q, 2H), 5.50 (dd, 1H), 6.00 (s, 1H), 6.30 (s, 1H), 6.90 (s, 1H), 8.71 (d, 2H), 8.85 (s, 1H), 11.45 (s, 1H); Mass Spectrum 448 [MH]+.

### Example 11

### 6-Ethyl-2-{2-[3-(2-cyanopyrid-3-yl)isoxazol-5-yl]azetidin-1-yl}-4-(5-methyl-1H-pyrazol-3-ylamino)pyrimidine

A mixture of 2-chloro-6-ethyl-4-(5-methyl-1H-pyrazol-3-ylamino)pyrimidine (146 mg, 0.62 mmol), S-2-[3-(2-cyanopyrid-3-yl)isoxazol-5-yl]azetidine (240 mg, lmmol) and DIPEA (0.3 ml, 1.7 mmol) in 1-hexanol (4 ml) was heated at 130° C. for 3 hours. The mixture was allowed to cool and the volatiles removed by evaporation. The residue was purified by chromatography on silica gel eluting with EtOAc/hexanes (5:95 increasing in polarity to 50:50). The purified product was triturated with DCM/hexanes, collected by filtration and dried to give the title compound (20 mg, 8%) as a racemate. NMR Spectrum 1.15 (t, 3H), 2.12 (s, 3H), 2.38-2.5 (m, 2H), 2.75-2.82 (m, 1H), 4.06-4.15 (m, 2H), 5.55 (t, 1H), 6.0 (s, 1H), 6.30 (s, 1H), 7.08 (s, 1H), 7.89 (dd, 1H), 8.39 (d, 1H), 8.80 (s, 1H), 8.90 (s, 1H), 11.5 (s, 1H); Mass Spectrum 428 [MH]+.

The S-2-[3-(2-cyanopyrid-3-yl)isoxazol-5-yl]azetidine starting material was prepared as follows:

A solution of hydroxylamine hydrochloride (533 mg, 7.6 mmol) in water (1.8 ml) was added drop-wise to sodium hydroxide (708 mg, 17mmol) in water (2 ml). The resulting solution was then added to a solution of 2-chloropyrid-3-ylcarboxaldehyde (1 g, 7 mmol) in ethanol (7 ml), water (7 ml) and ice (15 g). The mixture was stirred at ambient temperature for 18 hours. The mixture was neutralised to pH 7 with 6M hydrochloric acid. The solid product was collected by filtration, washed with water and dried to give 2-chloropyrid-3-ylcarboxaldehyde oxime (800 mg, 73%); NMR Spectrum 7.45 (dd, 1H), 8.18 (dd, 1H), 8.32 (s, 1H), 8.42 (dd, 1H); Mass Spectrum 157 [MH]+.

Sodium hypochlorite (10 ml of a 13% aqueous solution, 17.5 mmol), was added to a stirred mixture of 2-chloropyridine-3-carboxaldehdye oxime (1.56 g, 10 mmol) and S—N-tertbutoxycarbonyl-2-ethynylazetidine (2.0 g, 11 mmol) in DCM (60 ml) at 0° C. The mixture was allowed to warm to ambient temperature and stirred for 18 hours. The DCM layer was separated, dried (Na₂SO₄), and the solvent removed by evaporation. The residue was purified by chromatography on silica gel eluting with EtOAc/hexanes (25:75) to give S—N-(tert-butoxycarbonyl)-2-[3-(2-chloropyrid-3-yl)isoxazol-5-yl]azetidine (800 mg, 24%); NMR Spectrum 1.38 (s, 9H), 1.99 (s, 1H), 3.08-3.18 (m, 2H), 3.35-3.41 (m, 2H), 6.48 (s, 1H), 7.58 (dd, 1H), 7.64 (s, 1H), 8.18 (dd, 1H), 8.55 (dd, 1H); Mass Spectrum 336 [MH]+.

A mixture of S—N-(tert-butoxycarbonyl)-2-[3-(2-chloropyrid-3-yl)isoxazol-5-yl]azetidine (800 mg, 2.4 mmol), copper (I)cyanide (853 mg, 9.5 mmol), bis-palladium (0) tris-dibenzylideneacetone (87 mg, 0.095 mmol), 1,1′-bis (diphenylphosphino)ferrocene (311 mg, 0.38 mmol) and tetraethylammonium cyanide (372 mg, 2.38 mmol) in dry dioxane (15 ml) was thoroughly degassed by repeated evacuation and refilling with nitrogen and then the mixture was heated at reflux under nitrogen for 2 days. The mixture was allowed to cool and was diluted with EtOAc/methanol and insoluble matter was removed by filtration. The filtrate was washed with water and the organic layer separated, dried (MgSO₄) and the solvent removed by evaporation. The residue was purified by chromatography on silica gel eluting with EtOAc/hexane (30:70) to give S—N-(tert-butoxycarbonyl)-2-[3-(2-cyanopyrid-3-yl)isoxazol-5-yl]azetidine (338 mg, 49%).

A solution of 4M hydrogen chloride in dioxane (7 ml) was added to a solution of S—N-(tert-butoxycarbonyl)-2-[3-(2-cyanopyrid-3-yl)isoxazol-5-yl]azetidine (380 mg, 1.16 mmol) in methanol (7 ml) and the mixture stirred at ambient temperature for 18 hours. The volatiles were removed by evaporation and the residue dissolved in water, basified with aqueous ammonia and extracted with DCM. The extracts were combined, dried (Na₂SO₄) and the solvent removed by evaporation to give S-2-[3-(2-cyanopyrid-3-yl)isoxazol-5-yl]azetidine (240 mg, 91%).

### Example 12

### S-6-Methoxy-2-[2-{3-(2-methylpyrid-3-yl)isoxazol-5-yl]azetidin-1-yl1-4-(5-methyl-1H-pyrazol-3-ylamino)pvrimidine

A mixture of S-6-chloro-2-{2-[3-(2-methylpyrid-3-yl)isoxazol-5-yl]azetidin-1-yl}-4-(5-methyl-1H-pyrazol-3-ylamino)pyrimidine (55 mg, 0.13 mmol) and sodium methoxide (0.14 ml of a 25% solution in methanol) in dry methanol (4 ml) was heated at 120° C. under microwave irradiation for 3 hours. The mixture was allowed to cool and the volatiles removed by evaporation. The residue was dissolved in EtOAc/methanol, washed with water and dried (Na₂SO₄). The solvent was removed by evaporation and the residue purified by chromatography on silica gel eluting with EtOAc/hexanes (80:20 increasing to 100:0) to give the title compound (15 mg, 28%); NMR Spectrum 2.10 (s, 3H), 2.62 (s, 3H), 2.80-2.68 (m, 1H), 3.70 (s, 3H), 4.00-4.10 (m, 3H), 5.5 (t, 1H), 5.99 (s, 1H), 6.99 (s, 1H), 7.38 (dd, 1H), 7.96 (d, 1H), 8.55 (d, 1H), 9.20 (s, 1H), 11.75 (s, 1H); Mass Spectrum 419 [MH]+.

### Example 13

### S-6-Chloro-2-{2-[3-(3-ethylpyrazin-2-yl)isoxazol-5-yl]azetidin-1-yl}-4-(5-methyl-1H-pyrazol-3-ylamino)pyrimidine

A mixture of 2,6-dichloro-4-(5-methyl-1H-pyrazol-3-ylamino)pyrimidine (prepared according to Method 29 of WO2005/040159) (1.1 g, 4.5 mmol), S-2-[3-(3-ethylpyrazin-2-yl)isoxazol-5-yl]azetidine (1.15 g, 5 mmol) and DIPEA (2 ml) in 1-hexanol (20 ml) was heated at 75° C. for 18 hours. The 1-hexanol was removed by evaporation and the residue adsorbed onto silica and purified by chromatography on silica gel eluting with EtOAc/hexanes (1:1) to give the title compound (860 mg, 53%); NMR Spectrum 1.28 (t, 3H), 2.14 (s, 3H), 2.78-2.88 (m, 1H), 3.18 (q, 2H), 4.05-4.22 (m, 2H), 5.60 (dd, 1H), 6.03 (s, 1H), 6.48 (s, 1H), 6.96 (s, 1H), 8.55 (s, 1H), 8.64 (s, 1H), 9.33 (s, 1H), 11.62 (s, 1H); Mass Spectrum 438 [MH]+.

The S-2-[3-(3-ethylpyrazin-2-yl)isoxazol-5-yl]azetidine starting material was prepared as follows:

A mixture of 2-methyl-3-ethylpyrazine (22.35 g, 183 mmol), selenium dioxide (30 g, 270 mmol), and diatomaceous earth (30 g) in dioxane (250 ml) was heated at reflux for 18 hours. The mixture was allowed to cool and the solid material removed by filtration through diatomaceous earth. The filter pad was washed with methanol several times and the volatiles removed from the combined filtrate by evaporation. The residue was purified by chromatography on silica gel eluting with EtOAc/hexanes (0:100 increasing in polarity to 15:85) to give 3-ethylpyrazine-2-carboxaldehyde (3.0 g, 12%); NMR Spectrum 1.22 (t, 3H), 3.19 (q, 2H), 8.78 (s, 1H), 8.80 (s, 1H), 10.10 (s, 1H).

A solution of sodium hydroxide (4.52 g, 0.113mol) in water (15 ml) was added to a solution of hydroxylamine hydrochloride (3.48 g, 50 mmol) in water (15 ml). The mixture was cooled in an ice bath and then added carefully to a solution of 3-ethylpyrazine-2-carboxaldehyde (6.15 g, 45 mmol) in a mixture of ethanol (85 ml), water (85 ml), and ice (80 g). The mixture was stirred at ambient temperature for 18 hours and then neutralised to pH 7 with 6M hydrochloric acid. The mixture was concentrated by evaporation and the resulting precipitated product collected by filtration. The product was washed with water and dried under vacuum to give 3-ethylpyrazine-2-carboxaldehyde oxime (2.3 g, 34%); NMR Spectrum 1.20 (t, 3H), 3.05 (q, 2H), 8.25 (s, 1H), 8.52 (s, 2H), 11.89 (s, 1H); Mass Spectrum [MH]+.

Sodium hypochlorite (8 ml of a 13% aqueous solution, 14 mmol), was added to a stirred mixture of 3-ethylpyrazine-2-carboxaldehyde oxime (1.21 g, 8 mmol) and S—N-tertbutoxycarbonyl-2-ethynylazetidine (1.6 g, 8.8 mmol) in DCM (100 ml) at 0C. The mixture was allowed to warm to ambient temperature and stirred for 18 hours. The DCM layer was separated, dried (Na₂SO₄), and the solvent removed by evaporation. The residue was purified by chromatography on silica gel eluting with EtOAc/hexanes (25:75increasing in polarity to 50:50) to give S—N-(tert-butoxycarbonyl)-2-[3-(3-ethylpyrazin-2-yl)isoxazol-5-yl]azetidine (1.55 g, 59%).

A solution of 4M hydrogen chloride in dioxane (15 ml) was added to solution of S—N-(tert-butoxycarbonyl)-2-[3-(3-ethylpyrazin-2-yl)isoxazol-5-yl]azetidine (1.55 g, 4.7 mmol) in methanol (15 ml) and the mixture stirred at ambient temperature for 18 hours. The volatiles were removed by evaporation and the residue dissolved in water, basified with aqueous ammonia and extracted with DCM. The extracts were combined, dried (Na₂SO₄) and the solvent removed by evaporation to give S-2-[3-(3-ethylpyrazin-2-yl)isoxazol-5-yl]azetidine (1.1 g, 100%); Mass Spectrum 229 [M-H]-.

### Example 14

### S-6-Ethoxy-2-{2-[3-(3-ethylpyrazin-2-yl)isoxazol-5-yl]azetidin-1-yl}-4-(5-metyl-1H-pyrazol-3-ylamino)pyrimidine

A mixture of S-6-chloro-2-{2-[3-(3-ethylpyrazin-2-yl)isoxazol-5-yl]azetidin-1-yl}-4-(5-methyl-1H-pyrazol-3-ylamino)pyrimidine (160 mg, 0.36 mmol), and sodium ethoxide (176 mg, 2.6 mmol) in dry ethanol (20 ml) was heated at 120° C. under microwave irradiation for 7.5 hours. The mixture was allowed to cool and purified directly by reverse phase HPLC using a C18 column eluting with 1% aqueous ammonia/acetonitrile (55:45 decreasing in polarity to 35:65) to give the title compound (30 mg, 18%); NMR Spectrum 1.20 (m, 3H), 1.22 (t, 3H), 2.10 (s, 3H), 2.70-2.78 (m, 1H), 3.18 (q, 2H), 4.02-4.20 (m, 2H), 5.50 (t, 1H), 5.90-6.03 (m, 2H), 7.00 (s, 1H), 8.62 (s, 1H), 8.80 (s, 1H), 9.18 (s, 1H), 11.73 (s, 1H); Mass Spectrum 448 [MH]+.

### Example 15

### S-6-(2-Methoxyethoxy)-2-{2-[3-(3-ethylpyrazin-2-yl)isoxazol-5-yl]azetidin-1-yl}-4-(5-methyl-1H-pyrazol-3-ylamino)pyrimidine

Sodium hydride (105 mg, 2.2 mmol) was added to 2-methoxyethanol (20 ml) and the mixture was stirred for 10 minutes while being purged with nitrogen. S-6-chloro-2-{2-[3-(3-ethylpyrazin-2-yl)isoxazol-5-yl]azetidin-1-yl}-4-(5-methyl-1H-pyrazol-3-ylamino)pyrimidin (190 mg, 0.44 mmol) was added and the mixture heated at 120° C. in a sealed vessel under microwave irradiation for 4.5 hours. The mixture was allowed to cool, was diluted with water and extracted with EtOAc. The extracts were combined washed with water, dried (Na₂SO₄) and the solvent removed by evaporation. The residue was purified by chromatography on silica gel eluting with EtOAc/hexanes (90:10) to give the title compound (127 mg, 62%); NMR Spectrum 1.25 (t, 3H), 2.10 (s, 3H), 2.68-2.79 (m, 1H), 3.15 (q, 2H), 3.20 (s, 1H), 3.28 (s, 3H), 3.50 (s, 2H), 4.0-4.12 (m, 2H), 4.15-4.25 (s, 2H), 5.52 (t, 1H), 6.00 (s, 1H), 7.02 (s, 1H), 8.62 (s, 1H), 8.68 (s, 1H), 9.22 (s, 1H), 11.91 (s, 1H); Mass Spectrum 478 [MH]+.

### Example 16

### S-6-(2-Methoxyethoxy)-2-{2-[3-(2-methylpyrid-3-yl)isoxazol-5-yl]azetidin-1-yl}-4-(5-methyl-1H-pyrazol-3-ylamino)pyrimidine

Sodium hydride (140 mg, 3 mmol) was added to 2-methoxyethanol (20 ml) and the mixture was stirred for 10 minutes while being purged with nitrogen. S-6-chloro-2-{2-[3-(2-methylpyrid-3-yl)isoxazol-5-yl]azetidin-1-yl}-4-(5-methyl-1H-pyrazol-3-ylamino)pyrimidine (250 mg, 0.6 mmol) was added and the mixture heated at 120° C. in a sealed vessel under microwave irradiation for 3.5 hours. The mixture was allowed to cool, was diluted with water and extracted with EtOAc. The extracts were combined washed with water, dried (Na₂SO₄) and the solvent removed by evaporation. The residue was purified by chromatography on silica gel eluting with EtOAc/hexanes (90:10) to give the title compound (142 mg, 52%); NMR Spectrum 2.14 (s, 3H), 2.52-2.60 (m, 1H), 2.72-2.80 (m, 1H), 3.27 (s, 3H), 3.57 (dd, 2H), 4.05-4.14 (m, 2H), 4.25 (dd, 2H), 5.55 (dd, 1H), 5.85 (s, 1H), 5.94 (s, 1H), 6.84 (s, 1H), 7.31 (dd, 1H), 7.90 (d, 1H), 8.55 (d, 1H), 8.75 (s, 1H), 11.5 (s, 1H); Mass Spectru [MH]+.

### Example 17

### S-6-(3-Methoxypropoxy)-2-{2-[3-(3-ethylpyrazin-2-yl)isoxazol-5-yl]azetidin-1-yl}-4-(5-methyl-1H-pvrazol-3-ylamino)pyrimidine

Sodium hydride (109 mg, 2.28 mmol) was added to 3-methoxypropanol (20 ml) and the mixture was stirred for 10 minutes while being purged with nitrogen. S-6-chloro-2-{2-[3-(3-ethylpyrazin-2-yl)isoxazol-5-yl]azetidin-1-yl}-4-(5-methyl-1H-pyrazol-3-ylamino)pyrimid (200 mg, 0.46 mmol) was added and the mixture heated at 120° C. in a sealed vessel under microwave irradiation for 3.5 hours. The mixture was allowed to cool, was diluted with water and extracted with EtOAc. The extracts were combined washed with water, dried (Na₂SO₄) and the solvent removed by evaporation. The residue was purified by chromatography on silica gel eluting with EtOAc/hexanes (80:20 increasing in polarity to 100:0) to give the title compound (132 mg, 58%) as the racemate. The S enantiomer was separated by chiral HPLC using a Chiralpak AD column with methanol as eluent. NMR Spectrum 1.25 (t, 3H), 1.84 (q, 2H), 2.14 (s, 3H), 2.52-2.58 (m, 1H), 2.72-2.80 (m, 1H), 3.16 (q, 2H), 3.18 (s, 3H), 3.38 (t, 8.58 (s, 1H), 8.63 (s, 1H), 8.71 (s, 1H), 11.45 (s, 1H); Mass Spectrum 492 [MH]+.

### Example 18

### S-6-(3-Methoxypropoxy)-2-{2-[3-(2-methylpyrid-3-yl)isoxazol-5-yl]azetidin-1-yl}-4-(5-methyl-1H-pyrazol-3-ylamino)pyrimidine

Sodium hydride (102 mg, 2.36 mmol) was added to 3-methoxypropanol (20 ml) and the mixture was stirred for 10 minutes while being purged with nitrogen. S-6-chloro-2-{2-[3-(2-methylpyrid-3-yl)isoxazol-5-yl]azetidin-1-yl}-4-(5-methyl-1H-pyrazol-3-ylamino)pyrimidin (200 mg, 0.47 mmol) was added and the mixture heated at 120° C. in a sealed vessel under microwave irradiation for 2.5 hours. The mixture was allowed to cool, was diluted with water and extracted with EtOAc. The extracts were combined washed with water, dried (Na₂SO₄) and the solvent removed by evaporation. The residue was purified by chromatography on silica gel eluting with EtOAc/hexanes/methanol (50:50:0 increasing in polarity to 98:0:2) to give the racemate product. The S enantiomer was separated by chiral HPLC using a Chiralpak AD column with methanol as eluent to give the title compound (42 mg, 19%); NMR Spectrum 1.85 (q, 2H), 2.12 (s, 3H), 2.50-2.58 (m, 1H), 2.62 (s, 3H), 2.72-2.80 (m, 1H), 3.20 (s, 3H), 3.38 (t, 2H), 4.05-4.16 (m, 2H), 4.20 (t, 2H), 5.5 (dd, 1H), 5.87 (s, 1H), 5.95 (s, 1H), 6.81 (s, 1H), 7.30 (dd, 1H), 7.90 (d, 1H), 8.52 (d, 1H), 8.70 (s.1H), 11.5 (s, 1H); Mass Spectrum 477 [MH]+.

### Example 19

### S-6-Chloro-2-{2-[3-(3-methylpyrazin-2-yl)isoxazol-5-yl]azetidin-1-yl}-4-(5-methyl-1H-pyrazol-3-ylamino)pyrimidine

A mixture of 2,6-dichloro-4-(5-methyl-1H-pyrazol-3-ylamino)pyrimidine (Method 29 of WO2005/040159) (409 mg, 1.68 mmol), S-2-[3-(3-methylpyrazin-2-yl)isoxazol-5-yl]azetidine (400 mg, 1.85 mmol) and DIPEA (0.64 ml, 3.70 mmol) in 1-hexanol (20 ml) was heated at 75° C. for 18 hours. The 1-hexanol was removed by evaporation and the residue purified by chromatography on silica gel elating with EtOAc/hexanes (25:75 increasing in polarity to 100:0) to give the title compound (380 mg, 53%); NMR Spectrum 2.14 (s, 3H), 2.53-2.55 (m, 1H), 2.81 (s, 3H), 2.80-2.85 (m, 1H), 4.12-4.16 (m, 1H), 4.18-4.21 (m, 1H), 5.59-5.62 (m, 1H), 6.04 (s, 1H), 6.49 (s, 1H), 7.00 (d, 1H), 8.60-8.61 (m, 2H), 9.35 (s, 1H), 11.62 (s, 1H); Mass Spectrum 424 [MH]+.

The S-2-[3-(3-methylpyrazin-2-yl)isoxazol-5-yl]azetidine starting material was prepared as follows:

A mixture of 2,3-dimethylpyrazine (20 g, 18.5 mmol), selenium dioxide (41.06 g, 37 mmol) and diatomeous earth (20 g) in EtOAc (500 ml) was stirred and heated at 70° C. for 2 hours. The mixture was allowed to cool and the insoluble matter was removed by filtration through diatomeous earth. The filtrate was washed with saturated aqueous sodium hydrogen carbonate solution and then saturated aqueous sodium chloride solution, dried (MgSO₄) and the solvent removed by evaporation. The residue was suspended in water (100 ml) and hydroxylamine (45 ml of a 50% aqueous solution) was added. The mixture was stirred at ambient temperature for 18 hours and the mixture then extracted with EtOAc. The extracts were combined, washed with saturated aqueous sodium chloride solution, dried (MgSO₄) and the solvent removed by evaporation. The residue was triturated with isohexane to give 3-methylpyrazine-2-carboxaldehyde oxime (9.65 g, 38%); NMR Spectrum 2.67 (s, 3H), 8.23 (s, 1H), 8.45-8.49 (m, 2H), 11.87 (s, 1H).

Sodium hypochlorite (6.6 ml of a 13% aqueous solution, 9.5 mmol), was added to a stirred mixture of 3-methylpyrazine-2-carboxaldehyde oxime (1.0 g, 7.3 mmol) and S—N-tertbutoxycarbonyl-2-ethynylazetidine (1.92 g, 11.0 mmol) in DCM (50 ml) at 0° C. The mixture was stirred for 1 hour at 0° C. then allowed to warm to ambient temperature and stirred for 18 hours. The mixture was diluted with water and extracted with DCM. The extracts were combined, washed with a saturated solution of sodium chloride, dried (MgSO₄) and the solvent removed by evaporation. The residue was purified by chromatography on silica gel eluting first with DCM and then with EtOAc/hexanes (25:75) to give S—N-(tert-butoxycarbonyl)-2-[3-(3-methylpyrazin-2-yl)isoxazol-5-yl]azetidine (1.29 g, 56%); NMR Spectrum 1.40 (s, 9H), 2.00 (s, 1H), 2.45-2.53 (m, 1H), 2.63-2.72 (m, 1H), 2.94 (s, 3H), 3.99-4.05 (m, 1H), 4.06-4.11 (m, 1H), 5.36-5.39 (m, 1H), 6.90 (s, 1H), 8.50 (br s, 2H); Mass Spectrum 261 [M-C₄H₉]+.

A solution of 4M hydrogen chloride in dioxane (6 ml) was added to a solution of S—N-(tert-butoxycarbonyl)-2-[3-(3-methylpyrazin-2-yl)isoxazol-5-yl]azetidine (640 mg, 2.03 mmol) and in methanol (25 ml) and the mixture heated at 50° C. for 1 hour. The mixture was allowed to cool and the volatiles removed by evaporation. The residue dissolved in water (20 ml) and adjusted to pH 10 with concentrated aqueous ammonia and the mixture was extracted with DCM. The extracts were combined, dried (MgSO₄) and the solvent removed by evaporation to give S-2-[3-(3-methylpyrazin-2-yl)isoxazol-5-yl]azetidine (406 mg, 93%); NMR Spectrum (CDCl₃) 2.61-2.70 (m, 1H), 2.71-2.77 (m, 1H), 2.93 (s, 3H), 3.60-3.65 (m, 1H), 3.78 (q, 1H), 5.11 (t, 1H), 6.88 (s, 1H), 8.49-8.52 (m, 2H).

### Example 20

### S-6-(2-Methoxyethoxy)-2-{2-[3-(3-methylpyrazin-2-yl)isoxazol-5-yl]azetidin-1-yl}-4-(5-methyl-1H-pyrazol-3-ylamino)pyrimidine

Sodium hydride (99 mg of a 60% suspension in oil, 2.5 mmol) was added to 2-methoxyethanol (15 ml) and the mixture was stirred for 10 minutes while being purged with nitrogen. S-6-chloro-2-{2-[3-(3-methylpyrazin-2-yl)isoxazol-5-yl]azetidin-1-yl}-4-(5-methyl-1H-pyrazol-3-ylamino)pyrimidine (210 mg, 0.5 mmol) was added and the mixture heated at 120° C. in a sealed vessel under microwave irradiation for 3 hours. The mixture was allowed to cool, diluted with water and extracted with EtOAc. The extracts were combined washed with water and then saturated aqueous sodium chloride solution, dried (Na₂SO₄) and the solvent removed by evaporation. The residue was purified by chromatography on silica gel eluting with EtOAc/hexanes (25:75) to give the title compound (44 mg, 19%); NMR Spectrum 2.15 (s, 3H), 2.44-2.58 (m, 1H), 2.64-2.79 (m, 1H), 2.79 (s, 3H), 3.22 (s, 3H), 3.55-3.57 (m, 2H), 4.11-4.14 (m, 1H), 4.09-4.16 (m, 1H), 4.27-4.30 (m, 2H), 5.53-5.56 (m, 1H), 5.89 (s, 1H), 5.97 (s, 1H), 6.99 (d, 1H), 8.60 (s, 2H), 8.72 (s, 1H), 11.49 (s, 1H); Mass Spectrum 464 [MH]+.

### Example 21

### S-6-Chloro-2-{2-[3-(thiazol-2-yl)isoxazol-5-yl]azetidin-1-yl}-4-(5-methyl-1H-pyrazol-3-ylamino)pvrimidine

A mixture of 2,6-dichloro-4-(5-methyl-1H-pyrazol-3-ylamino)pyrimidine (prepared according to Method 29 of WO2005/040159) (1.35 g, 5.56 mmol), S-2-[3-(thiazol-2-yl)isoxazol-5-yl]azetidine (1.15 g, 5.56 mmol) and zinc acetate (0.51 g, 2.78 mmol) in isopropanol (60 ml) was heated at reflux for 18 hours. The mixture was allowed to cool and the volatiles removed by evaporation. The residue suspended in water and adjusted to pH9 with a 15% aqueous solution of sodium hydroxide. The aqueous mixture was extracted with EtOAc, washed with water and saturated aqueous sodium chloride solution, dried (MgSO₄) and the solvent removed by evaporation. The residue was purified by chromatography on silica gel, eluting with EtOAc/hexanes (50:50 increasing in polarity 100:0) to give the title compound (360 mg, 16%); NMR Spectrum 1.99 (s, 3H), 2.32-2.46 (m, 1H), 2.59-2.72 (m, 1H), 4.10-4.12 (m, 2H), 5.57-5.59 (m, 1H), 5.61 (s, 1H), 6.18 (s, 1H), 7.06 (s, 1H), 7.88 (d, 1H), 7.98 (d, 1H), 9.70 (s, 1H), 11.87 (s, 1H); Mass Spectrum 415 [MH]+.

The S-2-[3-(thiazol-2-yl)isoxazol-5-yl]azetidine starting material was prepared as follows:

A mixture of 2-acetylthiazole (5.0 g, 39 mmol), cyclohexylamine (5.85 g, 59 mmol) and p-toluenesulphonic acid (100 mg) in toluene was heated under Dean and Stark conditions for 48 hours. The mixture was allowed to cool and the volatiles removed by evaporation to give crude N-cyclohexyl[l-(thiazol-2-yl)ethylidene]amine (6.2 g, 76%) which was used without purification; NMR Spectrum (CDCl₃) 1.24-1.42 (m, 4H), 1.49-1.58 (m, 2H), 1.64-1.71 (m, 3H), 1.78-1.88 (m, 2H), 2.38 (s, 3H), 3.52-3.59 (m, 1H), 7.30 (d, 1H), 7.81 (d, 1H); Mass Spectrum 209 [MH+].

A solution of N-cyclohexyl[1-(thiazol-2-yl)ethylidene]amine (5.0 g, 24 mmol) in anhydrous THF (20 ml) was added over 10 minutes to a stirred solution of 2M lithium diisopropylamide in heptane (21 ml, 42 mmol) and anhydrous THF (50 ml) at -10° C. The mixture was stirred for 10 minutes and a solution of S-methyl N-tert-butyloxycarbonylazetidin-2-yl carboxylate (4.62 g, 21 mmol) in anhydrous THF (30 ml) was added over 35 minutes. The mixture was stirred at −10° C. for 30 minutes and allowed to warm to ambient temperature and stirred for a further 2 hours. Saturated aqueous ammonium chloride solution was added and the mixture extracted with EtOAc. The extracts were combined, washed with water and saturated aqueous sodium chloride solution, dried (MgSO₄) and the solvent removed by evaporation to give S-1-(N-tert-butyloxycarbonylazetidin-2-yl)-3-cyclohexylamino-3-(thiazol-2-yl)prop-2-en-1-one (2.86 g, 35%); NMR Spectrum 1.20-1.47 (m, 5H), 1.43 (s, 9H), 1.49-1.53 (m, 1H), 1.69-1.78 (m, 2H), 1.84-1.98 (m, 2H), 2.18-2.26 (m, 1H), 2.44-2.49 (m, 1H), 3.88-3.94 (m, 2H), 4.07-4.09 (m, 1H), 4.58-4.62 (m, 1H), 5.67 (s, 1H), 7.46 (d, 1H), 7.94 (d, 1H), 11.09 (d, 1H); Mass Spectrum 292 [M-C₄H₉]+.

A mixture of S-1-(N-tert-butyloxycarbonylazetidin-2-yl)-3-cyclohexylamino-3-(thiazol-2-yl)prop-2-en-1-one (2.86 g, 7.3mmol) and hydroxylamine hydrochloride (510 mg, 7.3 mmol) in THF (150 ml) was heated at reflux for 21 hours. The mixture was allowed to cool and the volatiles removed by evaporation. The residue was purified by chromatography on silica gel eluting with EtOAc/hexanes (25:75 increasing in polarity 100:0) to give S—N-(tert-butyloxycarbonyl)-2-[3-(thiazol-2-yl)-4,5-dihydro-5-hydroxyisoxazol-5-yl]azetidine (1.37g, 58%); NMR Spectrum (CDCl₃) 1.47-1.48 (m, 9H), 1.95-2.07 (m, 1H), 2.15-2.36 (m, 1H), 3.18 (d, 1H), 3.42 (d, 1H), 3.71-3.97 (m, 2H), 4.73 (d, 1H), 7.41 (d, 1H), 7.66 (s, 1H), 7.87 (d, 1H); Mass Spectrum 673 [2M+Na]+.

A solution of thionyl chloride (4.54 g, 38 mmol) in methyltetrahydrofuran (20 ml) was added at −20° C. to a mixture of S—N-(tert-butyloxycarbonyl)-2-[3-(thiazol-2-yl)-4,5-dihydro-5-hydroxyisoxazol-5-yl]azetidine (1.37 g, 4.22 mmol) and triethylamine (4.11 ml, 29.5 mmol) in methyltetrahydrofuran (80 ml). The mixture was then stirred for 10 minutes, quenched with water and then extracted with EtOAc. The extracts were combined, washed with water and then with saturated aqueous sodium chloride solution, dried (MgSO₄) and the solvent removed by evaporation to give S—N-(tert-butyloxycarbonyl)-2-[3-(thiazol-2-yl)isoxazol-5-yl]azetidine (1.32 g, 100%); NMR Spectrum (CDCl₃) 1.39 (s, 9H), 2.40-2.51 (m, 1H), 2.62-2.73 (m, 1H), 3.94-4.11 (m, 2H), 5.35 (t, 1H), 6.84 (s, 1H), 7.47 (s, 1H), 7.95 (s, 1H); Mass Spectrum 308 [MH]+.

TFA (10 ml) was added to a solution of S—N-(tert-butyloxycarbonyl)-2-[3-(thiazol-2-yl)isoxazol-5-yl]azetidine (1.32 g, 4.3 mmol) in DCM (80 ml) and the mixture stirred at ambient temperature for 60 hours. The mixture was concentrated by evaporation and diluted with water. The aqueous mixture was adjusted to pH10 by addition of sodium hydroxide solution and extracted with EtOAc. The extracts were combined, washed with brine, dried (MgSO₄) and the solvent removed by evaporation to give S-2-[3-(thiazol-2-yl)isoxazol-5-yl]azetidine (1.15 g, 100%) as an oil; NMR Spectrum (CDCl₃) 2.54 (s, 1H), 2.58-2.64 (m, 1H), 2.68-2.75 (m, 1H), 3.56-3.61 (m, 1H), 3.77 (q, 1H), 5.09 (t, 1H), 6.84 (s, 1H), 7.45 (d, 1H), 7.95 (d, 1H); Mass Spectrum 179 [MH-C₂H₅]+.

### Example 22

### S-6-(2-Methoxyethoxy)-2-{2-[3-(thiazol-2-yl)isoxazol-5-yl]azetidin-1-yl}-4-(5-methyl-1H-pyrazol-3-ylamino)pyrimidine

Sodium hydride (97 mg of a 60% suspension in oil, 2.42 mmol) was added to 2-methoxyethanol (18 ml) and the mixture was stirred for 10 minutes while being purged with nitrogen. S-6-Chloro-2-{2-[3-(thiazol-2-yl)isoxazol-5-yl]azetidin-1-yl}-4-(5-methyl-1H-pyrazol-3-ylamino)pyrimidine (200 mg, 0.48 mmol) was added and the mixture heated at 120° C. in a sealed vessel under microwave irradiation for 3 hours. The mixture was allowed to cool, was diluted with water and extracted with EtOAc. The extracts were combined washed with water and then saturated aqueous sodium chloride solution, dried (Na₂SO₄) and the solvent removed by evaporation. The residue was purified by chromatography on silica gel eluting with EtOAc/hexanes (50:50 increasing in polarity to 100:0). The purified product was triturated with hexane and collected by filtration to give the title compound (61 mg, 28%); as a partial racemate 67% ee; NMR Spectrum 2.15 (s, 3H), 2.45-2.57 (m, 1H), 2.76-2.80 (m, 1H), 2.94 (s, 1H), 3.24 (s, 3H), 3.56 (t, 2H), 4.08-4.15 (m, 2H), 4.26-4.30 (m, 2H), 5.51-5.54 (m, 1H), 5.88 (s, 1H), 5.95 (s, 1H), 6.98 (s, 1H), 7.90 (d, 1H), 8.04 (d, 1H), 8.77 (s, 1H), 11.51 (s, 1H); Mass Spectrum 455 [MH]+.

### Example 23

### S-6-(2-Methoxyethoxy)-2-{2-[3-(pyrid-2-yl)isoxazol-5-yl]azetidin-1-yl}-4-(5-methyl-1H-pyrazol-3-ylamino)pyrimidine

Sodium hydride (100 mg of a 60% suspension in oil, 2.5 mmol) was added to 2-methoxyethanol (18 ml) and the mixture was stirred for 10 minutes while being purged with nitrogen. S-6-chloro-2-{2-[3-(pyrid-2-yl)isoxazol-5-yl]azetidin-1-yl}-4-(5-methyl-1H-pyrazol-3-ylamino)pyrimidine (204 mg, 0.5 mmol) was added and the mixture heated at 120° C. in a sealed vessel under microwave irradiation for 3 hours. The mixture was allowed to cool, was diluted with water and extracted with EtOAc. The extracts were combined washed with water and then saturated aqueous sodium chloride solution, dried (Na₂SO₄) and the solvent removed by evaporation. The residue was purified by chromatography on silica gel eluting with EtOAc/hexanes (50:50 increasing in polarity to 100:0). The purified product was triturated with DCM/hexane and collected by filtration to give the title compound (33 mg, 15%); NMR Spectrum 2.15 (d, 3H), 2.46-2.55 (m, 1H), 2.72-2.81 (m, 1H), 3.22 (s, 3H), 3.50 (s, 1H), 3.56-3.58 (m, 2H), 4.09-4.16 (m, 2H), 4.27-4.30 (m, 2H), 5.50-5.54 (m, 1H), 5.88 (s, 1H), 5.97 (s, 1H), 6.99 (d, 1H), 7.47-7.50 (m, 1H), 7.92-7.96 (m, 1H), 7.99-8.02 (m, 1H), 8.69-8.71 (m, 1H), 8.78 (s, 1H); Mass Spectrum 449 [MH]+.

### Example 24

### S-2-{2-[3-(pyrid-2-yl)isoxazol-5-yl]azetidin-1-yl}-4-(5-tert-butyl-1H-pyrazol-3-ylamino)pyrimidine

A mixture of 2-chloro-4-(5-tert-butyl-1H-pyrazol-3-ylamino)pyrimidine (125 mg, 0.5 mmol), S-2-[3-(pyrid-2-yl)isoxazol-5-yl]azetidine (150 mg, 0.5 mmol) and DIPEA (0.3 ml, 1.7 mmol) in 1-hexanol (3 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (100 mg, 48%); NMR Spectrum 1.25 (s, 9H), 2.40-2.55 (m, 1H), 2.70-2.85 (m, 1H), 4.05-4.20 (m, 2H), 5.45-5.55 (t, 1H), 6.05-6.25 (br s, 1H), 6.25-6.45 (br s, 1H), 6.90 (s, 1H), 7.40-7.50 (m, 1H), 7.80-8.05 (m, 3H), 8.64-8.69 (d, 1H), 8.95-9.15 (br s, 1H), 11.45-11.70 (br s, 1H); Mass Spectrum 417 [MH]+.

The 2-chloro-4-(5-tert-butyl-1H-pyrazol-3-ylamino)pyrimidine starting material was prepared as follows:

A mixture of 2,4-dichloropyrimidine (3.73 g, 25 mmol) and 5-amino-3-tert-butyl-1H-pyrazole (3.55 g, 25.5 mmol) and DIPEA (4.44 ml, 25 mmol) were heated at 50° C. in THF (75 ml) for 18 hours tnder nitrogen. The mixture was allowed to cool and the volatiles removed by evaporation. The residue was dissolved in DCM (150 ml), washed with water, dried (MgSO₄) and the solvent removed by evaporation. The residue was purified by chromatography on silica gel eluting with EtOAc/hexanes (1:3) and then methanol/DCM (5:95) to give 2-chloro-4-(5-tert-butyl-1H-pyrazol-3-ylamino)pyrimidine (1.73 g, 28%). NMR Spectrum 1.30 (s, 9H), 6.05 (s, 1H), 7.16 (br s, 1H), 8.12 (d, 1H), 9.80 (br s, 1H), 11.89 (br s, 1H); Mass Spectrum 252 [MH]+.

### Example 25

### S-6-Ethyl-2-{2-[3-(Pyrid-2-yl)isoxazol-5-yl]azetidin-1-yl}-4-(5-methyl-1H-pyrazol-3-ylamino)pyrimidine

A mixture of 2-chloro-6-ethyl-4-(5-methyl-1H-pyrazol-3-ylamino)pyrimidine (118 mg, 0.5 mmol), S-2-[3-(pyrid-2-yl)isoxazol-5-yl]azetidine (150 mg, 0.5 mmol), DIPEA (0.3 ml, 1.7 mmol) in 1-hexanol (3 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (76mg, 38%); NMR Spectrum 1.10-1.18 (t, 3H), 2.10 (s, 3H), 2.40-2.50 (q, 2H), 2.45-2.55 (m, 1H), 2.70-2.80 (m, 1H), 4.00-4.20 (m, 2H), 5.45-5.55 (t, 1H), 5.95-6.15 (br s, 1H), 6.15-6.35 (br s, 1H), 6.95 (s, 1H), 7.45-7.55 (m, 1H), 7.88-7.95 (m, 1H), 7.95-8.05 (d, 1H), 8.64-8.70 (d, 1H), 8.75-8.95 (br s, 1H), 11.45-11.60 (br s, 1H); Mass Spectrum 403 [MH]+.

### Example 26

### S-2-{2-[3-(3-methylpyrazin-2-yl)isoxazol-5-yl]azetidin-1-yl}-4-(5-tert-butyl-1H-pyrazl-3-ylamino)pyrimidine

A mixture of 2-chloro-4-(5-tert-butyl-1H-pyrazol-3-ylamino)pyrimidine (152 mg, 0.6 mmol), S-2-[3-(3-methylpyrazin-2-yl)isoxazol-5-yl]azetidine (194 mg, 0.9 mmol) and DIPEA (0.3 ml, 1.7 mmol) in 1-hexanol (3 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (130 mg, 50%); NMR Spectrum 1.23 (s, 9H), 2.45-2.57 (m, 1H), 2.75-2.90 (m, 1H), 2.80 (s, 3H), 4.05-4.20 (m, 2H), 5.50-5.60 (t, 1H), 6.05-6.25 (br s, 1H), 6.25-6.45 (br s, 1H), 6.93 (s, 1H), 7.86-7.92 (d, 1H), 8.55-8.62 (m, 2H), 8.95-9.15 (br s, 1H), 11.45-11.70 (br s, 1H); Mass Spectrum 432 [MH]+.

### Example 27

### S-6-Ethyl-2-{2-[3-(3-methylpyrazin-2-yl)isoxazol-5-yl]azetidin-1-yl}-4-(5-methyl-1H-pyrazol-3-ylamino)pyrimidine

A mixture of 2-chloro-6-ethyl-4-(5-methyl-1H-pyrazol-3-ylamino)pyrimidine (142 mg 0.5 mmol), S-2-[3-(3-methylpyrazin-2-yl)isoxazol-5-yl]azetidine (208 mg, 0.9 mmol) and DIPEA (0.3 ml, 1.7 mmol) in 1-hexanol (3 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (110 mg, 53%); NMR Spectrum 1.10-1.18 (t, 3H), 2.10 (s, 3H), 2.40-2.50 (q, 2H), 2.45-2.55 (m, 1H), 2.70-2.80 (m, 1H), 2.80 (s, 3H), 4.00-4.20 (m, 2H), 5.45-5.55 (t, 1H), 5.95-6.15 (br s, 1H), 6.15-6.35 (br s, 1H), 6.95 (s, 1H), 8.55-8.60 (m, 2H), 8.75-8.95 (br s, 1H), 11.45-11.60 (br s, 1H); Mass Spectrum 418 [MH]+.

### Example 28

### S-6-Methyl-2-{2-[3-(3-methylpyrazin-2-yl)isoxazol-5-yl]azetidin-1-yl}-4-(5-tert-butyl-1H-pyrazol-3-ylamino)pyrimidine

A mixture of 2-chloro-6-methyl-4-(5-tert-butyl-1H-pyrazol-3-ylamino)pyrimidine (168 mg, 0.6 mmol), S-2-[3-(3-methylpyrazin-2-yl)isoxazol-5-yl]azetidine (194 mg, 0.9 mmol) and DIPEA (0.3 ml, 1.7 mmol) in 1-hexanol (3 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (120 mg, 54%); NMR Spectrum 1.23 (s, 9H), 2.20 (s, 3H), 2.45-2.57 (m, 1H), 2.75-2.90 (m, 1H), 2.80 (s, 3H), 4.05-4.20 (m, 2H), 5.50-5.60 (t, 1H), 6.00-6.20 (br s, 1H), 6.20-6.40 (br s, 1H), 6.93 (s, 1H), 7.86-7.92 (d, 2H), 8.55-8.62 (m, 2H), 8.80-9.00 (br s, 1H), 11.45-11.65 (br s, 1H); Mass Spectrum 446 [MH]+.

The 2-chloro-6-methyl-4-(5-tert-butyl-1H-pyrazol-3-ylamino)pyrimidine starting material was prepared as follows:

A mixture of 2,4-dichloro-6-methylpyrimidine (3.52 g, 22 mmol) and 5-amino-3-tert-butyl-1H-pyrazole (3.00 g, 22 mmol) and anhydrous sodium carbonate (2.29 g, 22 mmol) in ethanol (75 ml) was stirred at ambient temperature for 24 hours. The insoluble material was removed by filtration and the solvent removed from the filtrate by evaporation. The residue was purified by chromatography on silica gel eluting with EtOAc/isohexane (1:3) and then methanol/DCM (5:95) to give 2-chloro-6-methyl-4-(5-tert-butyl-1H-pyrazol-3-ylamino)pyrimidine (3.16 g, 55%); NMR Spectrum 1.31 (s, 9H), 2.19 (s, 3H), 6.15 (s, 1H), 7.25 (s, 1H), 10.13 (s, 1H), 12.14 (s, 1H); Mass Spectrum 266 [MH]+.

### Example 29

### S-6-Ethyl-2-{2-[3-(3-methoxypyrazin-2-yl)isoxazol-5-yl]azetidin-1-yl}-4-(5-methyl-1H-pyrazol-3-ylamino)pyrimidine

A mixture of 2-chloro-6-ethyl-4-(5-methyl-1H-pyrazol-3-ylamino)pyrimidine (142 mg, 0.6 mmol), S-2-[3-(3-methoxypyrazin-2-yl)isoxazol-5-yl]azetidine (208 mg, 0.9 mmol) and DIPEA (0.3 ml, 1.7 mmol) in 1-hexanol (3 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (110 mg, 51%); NMR Spectrum 1.10-1.18 (t, 3H), 2.10 (s, 3H), 2.35-2.45 (q, 2H), 2.45-2.55 (m, 1H), 2.70-2.80 (m, 1H), 3.95-4.15 (m, 2H), 4.05 (s, 3H), 5.45-5.55 (t, 1H), 5.95-6.15 (br s, 1H), 6.15-6.35 (br s, 1H), 6.95 (s, 1H), 8.30-8.40 (m, 2H), 8.75-8.95 (br s, 1H), 11.45-11.60 (br s, 1H); Mass Spectrum 434 [MH]+.

The S-2-[3-(3-methoxypyrazin-2-yl)isoxazol-5-yl]azetidine starting material was prepared as follows:

Sodium hypochlorite (15 ml of a 13% aqueous solution, 26 mmol) was added drop-wise over 2 hours to a solution of 3-methoxypyrazine-2-carboxaldehyde oxime (prepared according to Method 67 of WO2005/040159) (2.30 g, l5 mmol) and S—N-tertbutoxycarbonyl-2-ethynylazetidine (3.0 g, 16 mmole) in DCM (100 ml) at -10° C. The solution was allowed to warm to ambient temperature and stirred for 18 hours. The mixture was extracted with DCM, the extracts combined, washed with water and then brine, dried (Na₂SO₄) and volatiles removed by evaporation. The residue was purified by chromatography on silica gel eluting with EtOAc/hexanes (20:80 increasing in polarity to 40:60) to give S—N-(tert-butoxycarbonyl)-2-[3-(3-methoxypyrazin-2-yl)isoxazol-5-yl]azetidine (1.30 g, 26%) as an oil; NMR Spectrum (CDCl₃) 1.40 (s, 9H), 2.40-2.50 (m, 1H), 2.50-2.60 (m, 1H), 3.95-4.20 (m, 2H), 4.10 (s, 3H), 5.40-5.50 (t, 1H), 6.90 (s, 1H), 8.20 (d, 1H), 8.30 (d, 1H).

4M hydrogen chloride solution in dioxane (15 ml) was added to a solution of S—N-(tert-butoxycarbonyl)-2-[3-(3-methoxypyrazin-2-yl)isoxazol-5-yl]azetidine (1.3 g, 4 mmol) in methanol (100 ml) and the mixture heated at 50° C. for 4 hours. The volatiles were removed by evaporation and the residue diluted with water and basified with concentrated aqueous ammonia solution. The mixture was extracted with DCM, the extracts combined, washed with water and then brine, dried (Na₂SO₄) and volatiles removed by evaporation. The residue was purified by chromatography on silica gel eluting with Methanol/DCM (50:50) to give S-2-[3-(3-methoxypyrazin-2-yl)isoxazol-5-yl]azetidine (450 mg, 50%) as an oil; NMR Spectrum (CDCl₃) 2.55-2.65 (m, 1H), 2.65-2.75 (m, 1H), 3.55-3.65 (m, 1H), 3.70-3.80 (q, 1H), 4.10 (s, 3H), 5.05-5.15 (t, 1H), 6.90 (s, 1H), 8.17 (d, 1H), 8.27 (d, 1H).

### Example 30

### S-6-Methyl-2-{2-[3-(3-methoxypyrazin-2-yl)isoxazol-5-yl]azetidin-1-yl]-4-(5-tert-butyl-1H-pyrazol-3-ylamino)pyrimidine

A mixture of 2-chloro-6-methyl-4-(5-tert-butyl-1H-pyrazol-3-ylamino)pyrimidine (158 mg, 0.6 mmol), S-2-[3-(3-methoxypyrazin-2-yl)isoxazol-5-yl]azetidine (220 mg, 0.95 mmol) and DIPEA (0.3 ml, 1.7 mmol) in 1-hexanol (3 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (150 mg, 64%); NMR Spectrum 1.20 (s, 9H), 2.15 (s, 3H), 2.45-2.57 (m, 1H), 2.75-2.90 (m, 1H), 3.95-4.20 (m, 2H), 4.0 (s, 3H), 5.50-5.60 (t, 1H), 6.00-6.20 (br s, 1H), 6.20-6.40 (br s, 1H), 6.92 (s, 1H), 8.30-8.35 (m, 2H), 8.80-9.00 (br s, 1H), 11.45-11.65 (br s, 1H); Mass Spectrum 462 [MH]+.

### Example 31

### S-6-Ethyl-2-{2-[3-(pyrimid-2-yl)isoxazol-5-yl]azetidin-1-yl]-4-(5-methyl-1H-pyrazol-3-ylamino)pyrimidine

A mixture of 2-chloro-6-ethyl-4-(5-methyl-1H-pyrazol-3-ylamino)pyrimidine (50 mg, 0.22 mmol), S-2-[3-(pyrimid-2-yl)isoxazol-5-yl]azetidine (53 mg, 0.25 mmol) and DIPEA (0.31 ml, 1.7 mmol) in 1-hexanol (1 ml) was heated at 130° C. for 3 hours. The mixture was allowed to cool and the volatiles removed by evaporation. The residue was purified by chromatography on silica gel eluting with EtOAc/methanol (100:0 increasing in polarity to 95:5). The purified product was triturated with DCM/hexanes, collected by filtration and dried to give the title compound (23 mg, 52%); NMR Spectrum 1.10-1.18 (t, 3H), 2.10 (s, 3H), 2.35-2.45 (q, 2H), 2.45-2.55 (m, 1H), 2.70-2.80 (m, 1H), 4.00-4.20 (m, 2H), 5.45-5.55 (t, 1H), 5.95-6.15 (br s, 1H), 6.15-6.35 (br s, 1H), 6.97 (s, 1H), 7.52-7.57 (t, 1H), 8.80-8.95 (br s, 1H), 8.91-8.95 (d, 2H), 11.45-11.60 (br s, 1H); Mass Spectrum 404 [MH]+.

The S-2-[3-(pyrimid-2-yl)isoxazol-5-yl]azetidine starting material was prepared as follows:

Sodium hypochlorite (10 ml of a 13% aqueous solution, 17.3 mmol) was added drop-wise over 2 hours to a solution of pyrimidine-2-carbaldehyde oxime (prepared according to Khimiya Geterotsiklicheskikh Soedinenii (1972), 10, 1422-4) (1.23 g, 10 mmol) and S—N-tertbutoxycarbonyl-2-ethynylazetidine (2.0 g, 11 mmol) in DCM (100 ml) at −10° C. The mixture was allowed to warm to ambient temperature and stirred for 18 hours. The mixture was extracted with DCM, the extracts combined, washed with water and then brine, dried (Na₂SO₄) and the solvent removed by evaporation. The residue was purified chromatography on silica gel eluting with EtOAc/hexanes (20:80 increasing in polarity to 40:60) to give S—N-(tert-butoxycarbonyl)-2-[3-(pyrimid-2-yl)isoxazol-5-yl]azetidine (350 mg, 12%) as an oil; NMR Spectrum (CDCl₃) 1.1-1.5 (br s, 9H), 2.30-2.50 (m, 1H), 2.55-2.70 (m, 1H), 3.80-4.00 (m, 2H), 5.35-5.45 (t, 1H), 7.05 (s, 1H), 7.60-7.65 (t, 1H), 8.98-9.00 (d, 2H).

A solution of S—N-(tert-butoxycarbonyl)-2-[3-(pyrimid-2-yl)isoxazol-5-yl]azetidine (350 mg, 0.16 mmol) was stirred in TFA (5 ml) for 5 hours. The solution was concentrated by evaporation and water (50 ml) added and solution basified by addition of concentrated aqueous ammonia solution. The aqueous mixture was extracted with DCM, the extracts combined, washed with water and then brine, dried (Na₂SO₄) and volatiles removed by evaporation to give S-2-[3-(pyrimid-2-yl)isoxazol-5-yl]azetidine (150 mg, 64%); NMR Spectrum (CDCl₃) 2.55-2.75 (m, 2H), 3.55-3.65 (m, 1H), 3.7-3.8 (q, 1H), 6.93 (s, 1H), 7.30-7.35 (t, 1H), 8.83-8.88 (d, 2H).

### Example 32

### S-6-Chloro-2-{2-[3-(pyrimid-2-yl)isoxazol-5-yl]azetidin-1-yl}-4-(5-methyl-1H-pyrazol-3-ylamino)pyrimidine

A mixture of 2,6-dichloro-4-(5-methyl-1H-pyrazol-3-ylamino)pyrimidine (prepared according to Method 29 of WO2005/040159) (80 mg, 0.41 mmol), S-2-[3-(pyrimid-2-yl)isoxazol-5-yl]azetidine (150 mg, 0.75 mmol) and DIPEA (0.2 ml, 1.2 mmol) in 1-hexanol (5 ml) was heated at reflux for 18 hours. The 1-hexanol was removed by evaporation and the residue adsorbed onto silica and purified by chromatography on silica gel eluting with EtOAc/hexanes (50:50 increasing in polarity to 100:0). The purified product was triturated with DCM/hexane to give the title compound (30 mg, 23%); Mass Spectrum 410 [MH]+.

### Example 33

### 6-Ethoxy-2-{2-[3-(pyrimid-2-yl)isoxazol-5-yl]azetidin-1-yl}-4-(5-methyl-1H-pyrazol-3-ylamino)pyrimidine

A mixture of S-6-chloro-2-{2-[3-(pyrimid-2-yl)isoxazol-5-yl]azetidin-1-yl}-4-(5-methyl-1H-pyrazol-3-ylamino)pyrimidine (50 mg, 00.12 mmol) and powdered sodium ethoxide (30 mg, 0.44 mmol) in ethanol (4 ml) was heated in a sealed vessel at 120° C. under microwave irradiation for 3 hours. More sodium ethoxide (30 mg, 0.44 mmol) was added and the solution heated for a further 3 hours at 120° C. and then a further 3 hours at 150° C. in a sealed vessel under microwave irradiation. The mixture was allowed to cool and the volatiles removed by evaporation. The residue was purified by chromatography on silica gel eluting with EtOAc/hexanes (50:50). The purified product was triturated with DCM/hexane and collected by filtration to give the title compound (6 mg, 7%) as a racemate; NMR Spectrum 1.10-1.22 (t, 3H), 2.10 (s, 3H), 2.40-2.60 (m, 1H), 2.70-2.80 (m, 1H), 4.00-4.20 (m, 2H), 4.2-4.35 (q, 2H), 5.45-5.55 (t, 1H), 5.75-5.95 (br s, 1H), 5.95-6.05 (br s, 1H), 6.97 (s, 1H), 7.50-7.55 (t, 1H), 8.50-8.70 (br s, 1H), 8.91-8.95 (d, 2H), 11.35-11.50 (br s, 1H); Mass Spectrum 420 [MH]+.

1. A compound of formula (I): 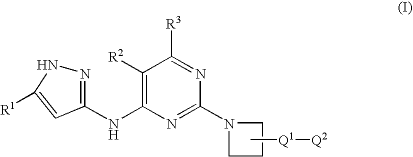
wherein:
R¹ is selected from a (C1-C6)alkyl, (C3-C8)cycloalkyl or (C3-C8)cycloalkyl(C1-C6)alkyl group, each of which groups may be optionally substituted by one or more substituents independently selected from halogeno and (C1-C6)alkoxy;
R² is selected from hydrogen, halogeno and trifluoromethyl;
R³ is selected from hydrogen, hydroxy and halogeno, or from a (C1-C6)alkyl, (C2-C6)alkenyl, (C2-C6)alkynyl, (C3-C8)cycloalkyl, (C3-C8)cycloalkyl(C1-C6)alkyl, (C1-C6)alkoxy, (C3-C8)cycloalkyl(C1-C6)alkoxy, (C1-C6)alkylcarbonyl, (C3-C8)cycloalkylcarbonyl, (C3-C8)cycloalkyl(C1-C6)alkylcarbonyl, (C1-C6)alkoxycarbonyl, amino, (C1-C6)alkylamino, di-[(C1-C6)alkyl]amino, (C3-C8)cycloalkylamino, (C3-C8)cycloalkyl(C1-C6)alkylamino, (C1-C6)alkoxyamino, carbamoyl, (C1-C6)alkylcarbamoyl, di-[(C1-C6)alkyl]carbamoyl, —C(O)R^{3b}, —OR^{3b}, —SR^{3b}, —NHR^{3b}, 13 N[(C1-C6)alkyl]R^{3b}, —S(O)ₘR^{3a} or —N(R^{3c})C(O)R^{3a} group, wherein R^{3a} is selected from a (C1-C6)alkyl, (C3-C8)cycloalkyl, (C3-C8)cycloalkyl(C1-C6)alkyl or (C1-C6)alkoxy group, m is 0, 1 or 2, R^{3b} is a saturated monocyclic 4-, 5- or 6-membered heterocyclic ring comprising at least one ring heteroatom selected from nitrogen, oxygen and sulfur and R^{3c} is selected from hydrogen and (C1-C6)alkyl,
or R³ is a saturated monocyclic 5- or 6-membered heterocyclic ring comprising at least one ring heteroatom selected from nitrogen, oxygen and sulfur,
or R³ is a 5- or 6-membered heteroaromatic ring comprising at least one ring heteroatom selected from nitrogen, oxygen and sulfur,
or R³ is a 2,7-diazaspiro[3.5]nonane group,
each of which groups or rings within R³ may be optionally substituted by one or more substituents independently selected from (C1-C6)alkyl, (C1-C6)alkoxy, (C1-C6)alkoxy(C1-C6)alkyl, (C1-C6)alkoxy(C1-C6)alkoxy, halogeno, hydroxy, trifluoromethyl, tri-[(C1-C4)alkyl]silyl, cyano, amino, (C1-C6)alkylamino, di-[(C1-C6)alkyl]amino, (C3-C8)cycloalkylamino, (C3-C6)cycloalkyl(C1-C3)alkylamino, amino(C1-C6)alkyl, (C1-C6)alkylamino(C1-C6)alkyl, di-[(C1-C6)alkyl]amino(C1-C6)alkyl, (C3-C8)cycloalkylamino(C1-C6)alkyl, (C3-C6)cycloalkyl(C1-C3)alkylamino(C1-C6)alkyl, (C1-C6)alkoxycarbonyl, carbamoyl, (C1-C6)alkylcarbamoyl, di-[(C1-C6)alkyl]carbamoyl, (C1-C6)alkylthio, (C1-C6)alkylsulfonyl, (C1-C6)alkylsulfinyl, (C1-C6)alkanoyl, an alkanoylamino group —N(R^{3d})C(O)R^{3e} wherein R^{3d} is selected from hydrogen and (C1-C6)alkyl and R^{3e} is selected from a (C1-C6)alkyl, (C3-C8)cycloalkyl, (C3-C8)cycloalkyl(C1-C6)alkyl or (C1-C6)alkoxy group, or a saturated monocyclic 3-, 4-, 5-, 6- or 7-membered ring, which ring may optionally comprise one or more heteroatoms selected from nitrogen, oxygen and sulfur, any of which substituents may be optionally substituted by one or more (C1-C4)alkyl, hydroxy or cyano groups;
Q¹ is a 5- or 6-membered heteroaromatic ring comprising at least one ring heteroatom selected from nitrogen, oxygen and sulfur, which ring is substituted by Q² and is optionally substituted, on any available ring atom, by one or more further substituents independently selected from (C1-C6)alkyl and (C1-C6)alkoxy (either of which (C1-C6)alkyl and (C1-C6)alkoxy substituent groups may be optionally substituted by one or more substituents independently selected from halogeno, amino, hydroxy and trifluoromethyl), halogeno, nitro, cyano, —NR⁴R⁵, carboxy, hydroxy, (C2-C6)alkenyl, (C3-C8)cycloalkyl, (C3-C8)cycloalkyl(C1-C6)alkyl, (C1-C4)al koxycarbonyl, (C1-C4)alkylcarbonyl, (C2-C6)alkanoylamino, phenylcarbonyl, —S(O)ₚ(C1-C4)alkyl, —C(O)NR⁶R⁷ and —SO₂NR⁸R⁹, wherein R⁴, R⁵, R⁶, R⁷, R⁸ and R⁹ are each independently selected from hydrogen and (C1-C6)alkyl, or R⁴ and R⁵, or R⁶ and R⁷, or R⁸ and R⁹, when taken together with the nitrogen atom to which they are attached, may each independently form a saturated heterocyclic ring and p is 0, 1 or 2;
Q² is selected from a (C1-C6)alkyl, (C3-C6)cycloalkyl or (C3-C6)cycloalkyl(C1-C6)alkyl group or a saturated or unsaturated 5- or 6-membered monocyclic ring which may comprise at least one ring heteroatom selected from nitrogen, oxygen and sulfur, and wherein Q² is optionally substituted by one or more substituents independently selected from (C1-C6)alkyl and (C1-C6)alkoxy (either of which (C1-C6)alkyl and (C1-C6)alkoxy substituent groups may be optionally substituted by one or more substituents independently selected from halogeno, amino, hydroxy and trifluoromethyl), halogeno, nitro, cyano, —NR¹⁰R¹¹, carboxy, hydroxy, (C2-C6)alkenyl, (C3-C8)cycloalkyl, (C1-C6)alkoxycarbonyl, (C1-C6)alkylcarbonyl, (C2-C6)alkanoylamino, phenylcarbonyl, —S(O)ₙ(C1-C6)alkyl, —C(O)NR¹²R¹³ and —SO₂NR¹⁴R¹⁵, wherein R¹⁰, R¹¹, R¹², R¹³, R¹⁴ and R¹⁵ are each independently selected from hydrogen and (C1-C6)alkyl, or R¹⁰ and R¹¹, or R¹² and R¹³, or R¹⁴ and R¹⁵, when taken together with the nitrogen atom to which they are attached, may each independently form a saturated heterocyclic ring and n is 0, 1 or 2;
and wherein any saturated monocyclic ring optionally bears 1 or 2 oxo or thioxo substituents;
or a pharmaceutically-acceptable salt thereof.

2. A compound of formula (I) according to claim 1, wherein R¹ is selected from a (C1-C6)alkyl or (C3-C8)cycloalkyl group, which group may be optionally substituted by one or more substituents selected from halogeno and (1-4C)alkoxy.

3. A compound of formula (I) according to claim 1, wherein R¹ is selected from methyl and tert-butyl.

4. A compound of formula (I) according to claim 3, wherein R¹ is methyl.

5. A compound of formula (I) according to claim 1, wherein R² is hydrogen.

6. A compound of formula (I) according to claim 1, wherein R³ is selected from hydrogen, hydroxy and halogeno, or from a (C1-C4)alkyl, (C2-C4)alkenyl, (C2-C4)alkynyl, (C1-C3)alkoxy, amino, (C1-C3)alkylamino, di-[(C1-C3)alkyl]amino, (C3-C6)cycloalkylamino, carbamoyl, (C1-C3)alkylcarbamoyl, di-[(C1-C3)alkyl]carbamoyl, —C(O)R^{3b}, —OR^{3b}, —NHR^{3b} or —S(O)ₘR^{3a} group, wherein R^{3a} is a (C1-C3)alkyl group, m is 0 and R^{3b} is a saturated monocyclic 4-, 5- or 6-membered heterocyclic ring comprising at least one ring heteroatom selected from nitrogen, oxygen and sulfur,
or R³ is a saturated monocyclic 5- or 6-membered heterocyclic ring comprising at least one ring heteroatom selected from nitrogen and oxygen,
or R³ is a 5- or 6-membered heteroaromatic ring comprising at least one ring heteroatom selected from nitrogen and oxygen,
each of which groups or rings within R³ may be optionally substituted by one or more substituents independently selected from (C1-C3)alkyl, (C1-C3)alkoxy, (C1-C3)alkoxy(C1-C3)alkyl, (C1-C3)alkoxy(C1-C3)alkoxy, halogeno, hydroxy, trifluoromethyl, amino, (C1-C3)alkylamino, di-[(C1-C3)alkyl]amino, amino(C1-C3)alkyl, carbamoyl, (C1-C3)alkylcarbamoyl, (C1-C3)alkylthio, (C1-C3)alkylsulfonyl, (C1-C3)alkanoyl, an alkanoylamino group —N(R^{3d})C(O)R^{3e} wherein R^{3d} is selected from hydrogen and (C1-C3)alkyl and R^{3e} is selected from a (C1-C3)alkyl or (C1-C3)alkoxy group, or a saturated monocyclic 3-, 4-, 5- or 6-membered ring, which ring may optionally comprise one or more heteroatoms selected from nitrogen, oxygen and sulfur, any of which substituents may be optionally substituted by one or more (C1-C2)alkyl, hydroxy or cyano groups, and wherein any saturated monocyclic ring within R³ optionally bears 1 oxo substituent.

7. A compound of formula (I) according to claim 1, wherein R³is selected from hydrogen and halogeno, or from a (C1-C4)alkyl or (C1-C3)alkoxy group,
or R³ is a saturated monocyclic 5- or 6-membered heterocyclic ring comprising at least one ring heteroatom selected from nitrogen and oxygen,
each of which groups or rings within R³ may be optionally substituted by one or more substituents independently selected from hydroxy and (C1-C3)alkoxy.

8. A compound of formula (I) according to claim 1, wherein R³is selected from hydrogen, chloro, methyl, ethyl, methoxy, ethoxy, 2-methoxyethoxy, 3-methoxyprop-1-yloxy and morpholino.

9. A compound of formula (I) according to claim 1, wherein R³is selected from methyl, ethyl, methoxy, ethoxy, 2-methoxyethoxy, 3-methoxyprop-1-yloxy and morpholino.

10. A compound of formula (I) according to claim 1, wherein Q¹ is a 5- or 6-membered heteroaromatic ring comprising one, two, three or four ring heteroatoms, which may be the same or different, selected from nitrogen, oxygen and sulfur, which ring is substituted by Q² and is optionally substituted, on any available ring atom, by one or more further substituents independently selected from (C1-C6)alkyl and (C1-C6)alkoxy (either of which (C1-C6)alkyl and (C1-C6)alkoxy substituent groups may be optionally substituted by one or more substituents independently selected from halogeno, amino, hydroxy and trifluoromethyl), halogeno, nitro, cyano, —NR⁴R⁵, carboxy, hydroxy, (C2-C6)alkenyl, (C3-C8)cycloal kyl, (C3-C8)cycloalkyl(C1-C6)alkyl, (C1-C4)alkoxycarbonyl, (C1-C4)alkylcarbonyl, (C2-C6)alkanoylamino, phenylcarbonyl, —S(O)ₚ(C1-C4)alkyl, —C(O)NR⁶R⁷ and —SO₂NR⁸R⁹, wherein R⁴, R⁵, R⁶, R⁷, R⁸ and R⁹ are each independently selected from hydrogen and (C1-C6)alkyl, or R⁴ and R⁵, or R⁶ and R⁷, or R⁸ and R⁹, when taken together with the nitrogen atom to which they are attached, may each independently form a saturated heterocyclic ring and p is 0, 1 or 2.

11. A compound of formula (I) according to claim 10, wherein Q¹ is selected from thienyl, pyrazolyl, oxazolyl, isoxazolyl, thiadiazolyl, pyrrolyl, furanyl, thiazolyl, triazolyl, tetrazolyl, imidazolyl, pyrazinyl, pyridazinyl, pyrimidinyl and pyridyl.

12. A compound of formula (I) according to claim 10, wherein Q¹ is a 5- or 6-membered heteroaromatic ring comprising a nitrogen and an oxygen ring heteroatom.

13. A compound of formula (I) according to claim 12, wherein Q¹ is isoxazolyl.

14. A compound of formula (I) according to claim 1, wherein Q² is an unsaturated 5- or 6-membered monocyclic ring comprising one or two ring heteroatoms independently selected from nitrogen, oxygen and sulfur, wherein Q² is optionally substituted by one or more substituents independently selected from (C1- C4)alkyl, (C1- C4)alkoxy and cyano.

15. A compound of formula (I) according to claim 1, wherein Q² is selected from pyrazinyl, pyrimidinyl, pyridyl and thiazolyl, wherein Q² is optionally substituted by one or more substituents independently selected from (C1-C4)alkyl, (C1-C4)alkoxy and cyano.

16. A compound of formula (I) selected from one or more of:
6-chloro-2-{2-[3-(pyrid-2-yl)isoxazol-5-yl]azetidin-1-yl}-4-(5-methyl-1H-pyrazol-3-ylamino)pyrimidine;
6-morpholino-2-{2-[3-(pyrid-2-yl)isoxazol-5-yl]azetidin-1-yl}-4-(5-methyl-1H-pyrazol-3-ylamino)pyrimidine;
6-methoxy-2-{2-[3-(pyrid-2-yl)isoxazol-5-yl]azetidi n-1-yl}-4-(5-methyl-1H-pyrazol-3-ylamino)pyrimidine;
6-methyl-2-{2-[3-(pyrid-2-yl )isoxazol-5-yl]azetidin-1-yl}-4-(5-methyl-1H-pyrazol-3-ylamino)pyrimidine;
S-6-ethyl-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(2-methylpyrid-3-yl)isoxazol-5-yl]azetidin-1-yl}-4-(5-methyl-1H-pyrazol-3-ylamino)pyrimidine;
S-6-chloro-2-{2-[3-(2-methylpyrid-3-yl)isoxazol-5-yl]azetidin-1-yl}-4-(5-methyl-1H-pyrazol-3-ylamino)pyrimidine;
S-6-morpholino-2-{2-[3-(2-methylpyrid-3-yl)isoxazol-5-yl]azetidin-1-yl}-4-(5-methyl-1H-pyrazol-3-ylamino)pyrimidine;
S-6-methyl-2-{2-[3-(3-ethoxypyrazin-2-yl)isoxazol-5-yl]azetidin-1-yl}-4-(5-methyl-1H-pyrazol-3-ylamino)pyrimidine;
S-6-ethyl-2-{2-[3-(3-ethoxypyrazi n-2-yl)isoxazol-5-yl]azetidin-1-yl}-4-(5-methyl-1H-pyrazol-3-ylamino)pyrimidine;
6-ethyl-2-{2-[3-(2-cyanopyrid-3-yl)isoxazol-5-yl]azetidin-1-yl}-4-(5-methyl-1H-pyrazol-3-ylamino)pyrimidine;
S-6-methoxy-2-{2-[3-(2-methylpyrid-3-yl)isoxazol-5-yl]azetidin-1-yl}-4-(5-methyl-1H-pyrazol-3-ylamino)pyrimidine;
S-6-chloro-2-{2-[3-(3-ethylpyrazin-2-yl)isoxazol-5-yl]azetidin-1-yl}-4-(5-methyl-1H-pyrazol-3-ylamino)pyrimidine;
S-6-ethoxy-2-{2-[3-(3-ethyl pyrazin-2-yl)isoxazol-5-yl]azetidi n-1-yl}-4-(5-methyl-1H-pyrazol-3-ylamino)pyrimidine;
S-6-(2-methoxyethoxy)-2-{2-[3-(3-ethylpyrazi n-2-yl)isoxazol-5-yl]azetidin-1-yl}-4-(5-methyl-1H-pyrazol-3-ylamino)pyrimidine;
S-6-(2-methoxyethoxy)-2-{2-[3-(2-methylpyrid-3-yl)isoxazol-5-yl]azetidin-1-yl}-4-(5-methyl-1H-pyrazol-3-ylamino)pyrimidine;
S-6-(3-methoxypropoxy)-2-{2-[3-(3-ethylpyrazin-2-yl)isoxazol-5-yl]azetidin-1-yl}-4-(5-methyl-1H-pyrazol-3-ylamino)pyrimidine;
S-6-(3-methoxypropoxy)-2-{2-[3-(2-methylpyrid-3-yl)isoxazol-5-yl]azetidin-1-yl}-4-(5-methyl-1H-pyrazol-3-ylamino)pyrimidine;
S-6-chloro-2-{2-[3-(3-methylpyrazin-2-yl)isoxazol-5-yl]azetidin-1-yl}-4-(5-methyl-1H-pyrazol-3-ylamino)pyrimidine;
S-6-(2-methoxyethoxy)-2-{2-[3-(3-methylpyrazin-2-yl)isoxazol-5-yl]azetidin-1-yl}-4-(5-methyl-1H-pyrazol-3-ylamino)pyrimidine;
S-6-chloro-2-{2-[3-(thiazol-2-yl)isoxazol-5-yl]azetidin-1-yl}-4-(5-methyl-1H-pyrazol-3-ylamino)pyrimidine;
S-6-(2-methoxyethoxy)-2-{2-[3-(thiazol-2-yl)isoxazol-5-yl]azetidin-1-yl}-4-(5-methyl-1H-pyrazol-3-ylamino)pyrimidine;
S-6-(2-methoxyethoxy)-2-{2-[3-(pyrid-2-yl)isoxazol-5-yl]azetidin-1-yl}-4-(5-methyl-1H-pyrazol-3-ylamino)pyrimidine;
S-2-{2-[3-(pyrid-2-yl )isoxazol-5-yl]azetidi n-1-yl}-4-(5-tert-butyl-1H-pyrazol-3-ylamino)pyrimidine;
S-6-ethyl-2-{2-[3-(pyrid-2-yl)isoxazol-5-yl]azetidin-1-yl}-4-(5-methyl-1H-pyrazol-3-ylamino)pyrimidine;
S-2-{2-[3-(3-methylpyrazin-2-yl)isoxazol-5-yl]azetidin-1-yl}-4-(5-tert-butyl-1H-pyrazol-3-ylamino)pyrimidine;
S-6-ethyl-2-{2-[3-(3-methyl pyrazin-2-yl)isoxazol-5-yl]azetidin-1-yl}-4-(5-methyl-1H-pyrazol-3-ylamino)pyrimidine;
S-6-methyl-2-{2-[3-(3-methylpyrazin-2-yl)isoxazol-5-yl]azetidin-1-yl}-4-(5-tert-butyl-1H-pyrazol-3-ylamino)pyrimidine;
S-6-ethyl-2-{2-[3-(3-methoxypyrazin-2-yl)isoxazol-5-yl]azetidin-1-yl}-4-(5-methyl-1H-pyrazol-3-ylamino)pyrimidine;
S-6-methyl-2-{2-[3-(3-methoxypyrazin-2-yl)isoxazol-5-yl]azetidin-1-yl}-4-(5-tert-butyl-1H-pyrazol-3-ylamino)pyrimidine;
S-6-ethyl-2-{2-[3-(pyrimid-2-yl)isoxazol-5-yl]azetidin-1-yl}-4-(5-methyl-1H-pyrazol-3-ylamino)pyrimidine;
S-6-chloro-2-{2-[3-(pyrimid-2-yl )isoxazol-5-yl]azetidin-1-yl}-4-(5-methyl-1H-pyrazol-3-ylamino)pyrimidine; and
6-ethoxy-2-{2-[3-(pyrimid-2-yl)isoxazol-5-yl]azetidin-1-yl}-4-(5-methyl-1H-pyrazol-3-ylamino)pyrimidine;
and pharmaceutically-acceptable salts thereof.

17. A pharmaceutical composition which comprises a compound of formula (I), or a pharmaceutically-acceptable salt thereof, according to claim 1 in association with a pharmaceutically-acceptable adjuvant, diluent or carrier.

18-21. (canceled)

22. (canceled)

23. A method for treating a disease or medical condition mediated alone or in part by IGF-1 R tyrosine kinase in a warm-blooded animal in need of such treatment, which comprises administering to said animal an effective amount of a compound of formula (I), or a pharmaceutically-acceptable salt thereof, according to claim 1.

24-27. (canceled)

28. A process for the preparation of a compound of formula (I), or a pharmaceutically-acceptable salt thereof, according to claim 1 which comprises:
(a) the reaction, conveniently in the presence of a suitable base, of a compound of formula (II): 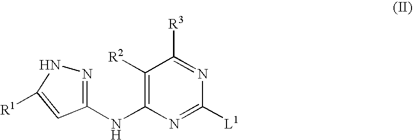
wherein L¹ represents a suitable displaceable group and R¹, R² and R³ are as defined in claim 1 except that any functional group is protected if necessary, with a compound of formula (III): 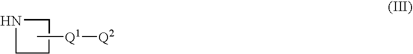
wherein Q¹ and Q² are as defined in claim 1 except that any functional group is protected if necessary; or
(b) the reaction, conveniently in the presence of a suitable acid, of a compound of formula (IV): 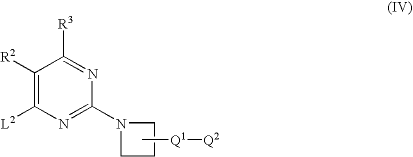
wherein L² is a suitable displaceable group and R², R³, Q¹ and Q² are as defined in claim 1 except that any functional group is protected if necessary, with a pyrazole of formula (V): 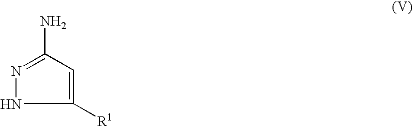
wherein R¹ is as defined in claim 1 except that any functional group is protected if necessary; or
(c) the reaction, conveniently in the presence of a suitable base, of a compound of formula (VI): 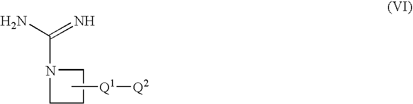
wherein Q¹ and Q² are as defined in claim 1 except that any functional group is protected if necessary, with a compound of formula (VII): 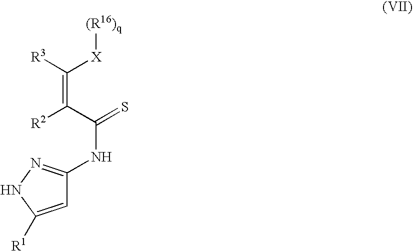
wherein X represents an oxygen atom and q is 1 or X represents a nitrogen atom and q is 2, R¹⁶ is a (C1-C6)alkyl group and R¹, R² and R³ are as defined in claim 1 except that any functional group is protected if necessary; or
(d) the reaction of a compound of formula (VIII): 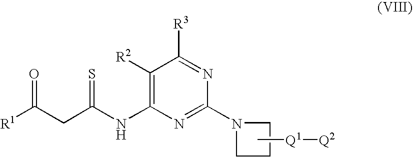
wherein R¹, R², R³, Q¹ and Q² are as defined in claim 1 except that any functional group is protected if necessary, with hydrazine; or
(e) for compounds of formula (I) wherein R³ is a (C1-C6)alkoxy, amino, (C1-C6)alkylamino, di-[(C1-C6)alkyl]amino, —OR^{3b}, —SR^{3b}, —NHR^{3b}, —N[(C1-C6)alkyl]R^{3b} or —S(O)ₘR^{3a} group wherein m is 0 and R^{3a} and R^{3b} are as defined in claim 1 (and the group R³ is optionally substituted by at least one group as defined in claim 1), the reaction, conveniently in the presence of a suitable base, of a compound of formula (IX): 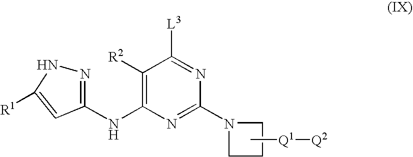
wherein L³ is a suitable displaceable group and R¹, R², Q¹ and Q² are as defined in claim 1 except that any functional group is protected if necessary, with a compound of formula:
H—Xa
wherein Xa represents OR¹⁷, NH₂, NHR¹⁷, N(R¹⁷)₂, OR^{3b}, SR^{3b}, NHR^{3b}, N[(C1- C6)alkyl]R^{3b} and SR^{3a}, wherein R¹⁷ is an, optionally substituted, (C1-C6)alkyl group and R^{3a} and R^{3b} are each as defined in claim 1 except that any functional group is protected if necessary; or
(f) for compounds of formula (I) wherein R³ is (i) an, optionally substituted, saturated monocyclic 5- or 6-membered heterocyclic ring comprising at least one ring nitrogen and, optionally, one or more additional heteroatoms selected from nitrogen, oxygen and sulfur, or (ii) an optionally substituted 2,7-diazaspiro[3.5]nonane group, the reaction, conveniently in the presence of a suitable base, of a compound of formula (IX): 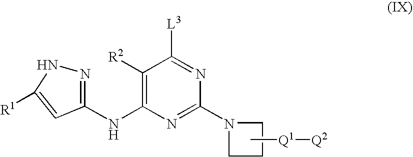
wherein L³ is a suitable displaceable group and R¹, R², Q¹ and Q² are as defined in claim 1 except that any functional group is protected if necessary, with (i) a compound of formula (Xb): 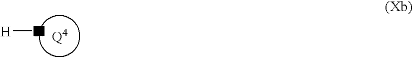
wherein Q⁴ is a saturated monocyclic 5- or 6-membered heterocyclic ring optionally comprising one or more heteroatoms selected from nitrogen, oxygen and sulfur in addition to the nitrogen, which ring is optionally substituted by at least one group as defined in claim 1, or with (ii) an optionally substituted 2,7-diazaspiro[3.5]nonane; or
(g) for compounds of formula (I) wherein R³ is a (C2-C6)alkenyl or (C2-C6)alkynyl group, and the group R³ is optionally substituted by at least one group as defined in claim 1, the reaction, conveniently in the presence of a suitable base and a suitable catalyst, of a compound of formula (IX): 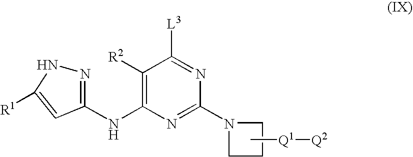
wherein L³ is a suitable displaceable group and R¹, R², Q¹ and Q² are as defined in claim 1 except that any functional group is protected if necessary, with a compound of formula (Xc) or of formula (Xc′): 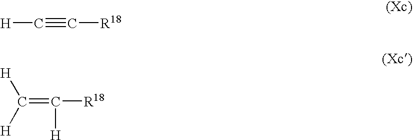
wherein R¹⁸ is selected from hydrogen and an, optionally substituted, (1-4C)alkyl or (C1-C4)alkoxycarbonyl group; or
(h) for compounds of formula (I) wherein R³ is attached to the pyrimidine ring through a carbon atom, the reaction, conveniently in the presence of a suitable catalyst, of a compound of formula (IX): 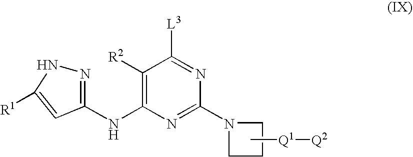
wherein L³ is a suitable displaceable group and R¹, R², Q¹ and Q² are as defined in claim 1 except that any functional group is protected if necessary, with a compound of the formula:
M-R³
wherein R³ is appropriately selected from the R³ groups as defined in claim 1 and M is a metallic group; or
(i) for compounds of formula (I) wherein R³ is a (C1-C6)alkoxycarbonyl group (and the group R³ is optionally substituted by at least one group as defined in claim 1), the reaction, conveniently in the presence of a suitable acid, of a compound of formula (X): 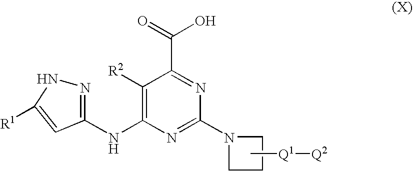
wherein R¹, R², Q¹ and Q² are as defined in claim 1 except that any functional group is protected if necessary, with a compound of formula:
H—O—(C1-C6)alkyl
wherein the (C1-C6)alkyl group is optionally substituted by at least one group as defined in claim 1 as a substituent for R³ and any functional group is protected if necessary; or
(j) for compounds of formula (I) wherein R³ is a 5-membered heteroaromatic ring comprising at least one heteroatom selected from nitrogen, oxygen and sulfur (and the group R³ is optionally substituted by at least one group as defined in claim 1), an internal condensation reaction using an appropriate starting material and a suitable dehydrating agent; or
(k) for compounds of formula (I) wherein R³ is a (C1-C6)alkyl, (C3-C6)alkenyl, (C3-C6)alkynyl or (C1-C6)alkoxy group substituted by at least one group as defined in claim 1, reacting a compound of formula (XII): 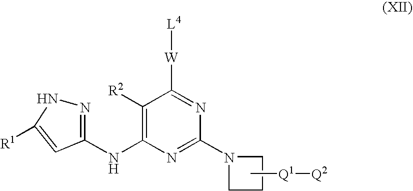
wherein L⁴ is a suitable displaceable group, W is an optionally substituted (C1-C6)alkyl, (C3-C6)alkenyl, (C3-C6)alkynyl or (C1-C6)alkoxy group and R¹, R², Q¹ and Q² are as defined in claim 1 except that any functional group is protected if necessary, with a compound of formula H—Xa, (Xb), (Xc), (Xc′) or M-R³;
and optionally after process (a), (b), (c), (d) (e), (f), (g), (h), (i), (j) or (k) carrying out one or more of the following:
converting the compound obtained to a further compound of the invention
forming a pharmaceutically-acceptable salt of the compound.
